SEQUENCE LISTING (46 sequences)

SEQ ID NO: 1 - DNA, 34 bp, Artificial Sequence
  Synthetic
gaagttcctattctctagaaagtataggaacttc

SEQ ID NO: 2 - DNA, 34 bp, Artificial Sequence
  Synthetic
  misc_feature (14)..(16): n is a, c, g, or t
  misc_feature (19)..(21): n is a, c, g, or t
ataacttcgtatannntannntatacgaagttat

SEQ ID NO: 3 - DNA, 23 bp, Artificial Sequence
  Synthetic
cctgcttttttatactaacttga

SEQ ID NO: 4 - DNA, 23 bp, Artificial Sequence
  Synthetic
tatccagtcaccccgccatgagg

SEQ ID NO: 5 - DNA, 23 bp, Artificial Sequence
  Synthetic
ccagtcaccccgccatgaggtga

SEQ ID NO: 6 - DNA, 23 bp, Artificial Sequence
  Synthetic
ccccgccatgaggtgacagagat

SEQ ID NO: 7 - DNA, 23 bp, Artificial Sequence
  Synthetic
cccgccatgaggtgacagagatg

SEQ ID NO: 8 - DNA, 23 bp, Artificial Sequence
  Synthetic
ccgccatgaggtgacagagatgg

SEQ ID NO: 9 - DNA, 23 bp, Artificial Sequence
  Synthetic
ccgccatgaggtgacagagatgg

SEQ ID NO: 10 - DNA, 23 bp, Artificial Sequence
  Synthetic
cgccatgaggtgacagagatggg

SEQ ID NO: 11 - DNA, 23 bp, Artificial Sequence
  Synthetic
ccatgaggtgacagagatgggac

SEQ ID NO: 12 - DNA, 23 bp, Artificial Sequence
  Synthetic
ctgagatgtaaaccaatttcagg

SEQ ID NO: 13 - DNA, 23 bp, Artificial Sequence
  Synthetic
ccaatttcaggccacaactccct

SEQ ID NO: 14 - DNA, 23 bp, Artificial Sequence
  Synthetic
caggccacaactcccttttctgg

SEQ ID NO: 15 - DNA, 23 bp, Artificial Sequence
  Synthetic
ccacaactcccttttctggtaca

SEQ ID NO: 16 - DNA, 23 bp, Artificial Sequence
  Synthetic
cccttttctggtacagacagacc

SEQ ID NO: 17 - DNA, 23 bp, Artificial Sequence
  Synthetic
ccttttctggtacagacagacca

SEQ ID NO: 18 - DNA, 23 bp, Artificial Sequence
  Synthetic
ggtacagacagaccatgatgcgg

SEQ ID NO: 19 - DNA, 23 bp, Artificial Sequence
  Synthetic
gtacagacagaccatgatgcggg

SEQ ID NO: 20 - DNA, 23 bp, Artificial Sequence
  Synthetic
tacagacagaccatgatgcgggg

SEQ ID NO: 21 - DNA, 23 bp, Artificial Sequence
  Synthetic
acagaccatgatgcggggactgg

SEQ ID NO: 22 - DNA, 23 bp, Artificial Sequence
  Synthetic
ccatgatgcggggactggagttg

SEQ ID NO: 23 - DNA, 23 bp, Artificial Sequence
  Synthetic
aacgttccgatagatgattcagg

SEQ ID NO: 24 - DNA, 23 bp, Artificial Sequence
  Synthetic
acgttccgatagatgattcaggg

SEQ ID NO: 25 - DNA, 23 bp, Artificial Sequence
  Synthetic
ccgatagatgattcagggatgcc

SEQ ID NO: 26 - DNA, 23 bp, Artificial Sequence
  Synthetic
agatgattcagggatgcccgagg

SEQ ID NO: 27 - DNA, 23 bp, Artificial Sequence
  Synthetic
cccgaggatcgattctcagctaa

SEQ ID NO: 28 - DNA, 23 bp, Artificial Sequence
  Synthetic
ccgaggatcgattctcagctaag

SEQ ID NO: 29 - DNA, 23 bp, Artificial Sequence
  Synthetic
cctaatgcatcattctccactct

SEQ ID NO: 30 - DNA, 23 bp, Artificial Sequence
  Synthetic
ccactctgaagatccagccctca

SEQ ID NO: 31 - DNA, 23 bp, Artificial Sequence
  Synthetic
agatccagccctcagaacccagg

SEQ ID NO: 32 - DNA, 23 bp, Artificial Sequence
  Synthetic
gatccagccctcagaacccaggg

SEQ ID NO: 33 - DNA, 23 bp, Artificial Sequence
  Synthetic
ccagccctcagaacccagggact

SEQ ID NO: 34 - DNA, 23 bp, Artificial Sequence
  Synthetic
ccctcagaacccagggactcagc

SEQ ID NO: 35 - DNA, 23 bp, Artificial Sequence
  Synthetic
cctcagaacccagggactcagct

SEQ ID NO: 36 - DNA, 23 bp, Artificial Sequence
  Synthetic
cccagggactcagctgtgtactt

SEQ ID NO: 37 - DNA, 23 bp, Artificial Sequence
  Synthetic
ccagggactcagctgtgtacttc

SEQ ID NO: 38 - DNA, 23 bp, Artificial Sequence
  Synthetic
cctccaggctgtgagcacagata

SEQ ID NO: 39 - DNA, 23 bp, Artificial Sequence
  Synthetic
ccaggctgtgagcacagatacgc

SEQ ID NO: 40 - DNA, 23 bp, Artificial Sequence
  Synthetic
agcacagatacgcagtattttgg

SEQ ID NO: 41 - DNA, 23 bp, Artificial Sequence
  Synthetic
gatacgcagtattttggcccagg

SEQ ID NO: 42 - DNA, 23 bp, Artificial Sequence
  Synthetic
agtattttggcccaggcacccgg

SEQ ID NO: 43 - DNA, 23 bp, Artificial Sequence
  Synthetic
cccaggcacccggctgacagtgc

SEQ ID NO: 44 - DNA, 23 bp, Artificial Sequence
  Synthetic
ccaggcacccggctgacagtgct

SEQ ID NO: 45 - DNA, 20 bp, Artificial Sequence
  Synthetic
aggccacaactcccttttct

SEQ ID NO: 46 - DNA, 20 bp, Artificial Sequence
  Synthetic
ggctggatcttcagagtgga

(19) United States
(12) Patent Application Publication
(10) Pub. No.: US 2019/0264193 A1
(43) Pub. Date: Aug. 29, 2019

(54) PROTEIN ENGINEERING METHODS

(71) Applicant: Caribou Biosciences, Inc., Berkeley, CA (US)

(72) Inventors: Shawdee Eshghi, Oakland, CA (US); Elizabeth Garner, Oakland, CA (US); Andrew P. May, San Francisco, CA (US); Megan Van Overbeek, Oakland, CA (US); Euan M. Slorach, Oakland, CA (US); Katharina Friederike Sonja Stengel, Oakland, CA (US)

(73) Assignee: Caribou Biosciences, Inc., Berkeley, CA (US)

(21) Appl. No.: 16/270,591

(22) PCT Filed: Aug. 11, 2017
(86) PCT No.: PCT/US2017/046631

§ 371 Date: Feb. 7, 2019

Related U.S. Application Data
(60) Provisional application No. 62/374,547, filed on Aug. 12, 2016.

(51) Int. Cl. C12N 15/10

(52) U.S. Cl. CPC C12N 15/1024; C12N 2310/20; C12N 15/1058

(57) ABSTRACT

Methods for preparing DNA libraries for protein engineering are described. The methods use programmable nucleases, such as from Clustered Regularly Interspaced Short Palindromic Repeats (CRISPR) systems, to generate diverse protein engineering libraries.

### CROSS-REFERENCE TO RELATED APPLICATIONS

This application claims the benefit of U.S. Provisional Patent Application Ser. No. 62/374,547, filed 12 Aug. 2016, now pending, which application is herein incorporated by reference in its entirety.

### STATEMENT REGARDING FEDERALLY SPONSORED RESEARCH OR DEVELOPMENT

Not applicable.

### SEQUENCE LISTING

The present application contains a Sequence Listing that has been submitted electronically in ASCII format and is hereby incorporated by reference in its entirety. The ASCII copy, created on 11 Aug. 2017 is named CBI023-30 ST25.txt and is 8 KB in size.

### TECHNICAL FIELD

The present invention relates to protein engineering methods. More particularly, the invention is directed to the use of programmable nucleases to generate diverse libraries for protein engineering.

### BACKGROUND OF THE INVENTION

Protein engineering provides a mechanism for designing proteins with new and/or desirable functions. Protein engineering methods include rational design approaches, such as site-directed mutagenesis. However, in many cases, there is limited information on the structure and mechanisms of the protein of interest. Thus, the use of random mutagenesis methods, including evolutionary-based methods such as DNA shuffling, have been developed.

Natural evolution results from genetic mutations that produce variants of a given protein within organisms. Variant proteins best adapted to a specific setting or purpose are then naturally selected. The production of antibodies is illustrative of this process. The natural antibody repertoire of an organism includes a large number of variants that are sampled by the organism and those that bind best to a specific antigen are selected from this natural library.

Assisted evolution, on the other hand, refers to the production of mutant libraries that encode protein variants. Such libraries can be used to identify structurally and functionally critical residues in a protein and to manipulate protein function. These mutant libraries can be screened for new proteins that show enhanced expression levels, solubility, stability, enzymatic activity, and/or interaction with desired binding partners. Such engineered proteins are important as therapeutics, diagnostics, and imaging agents in biological systems.

In DNA shuffling methods, a group of genes with double-stranded DNA and similar sequences is obtained from various organisms or produced by error-prone PCR. Digestion of these genes with DNasel yields randomly cleaved small fragments, which are purified and reassembled by PCR, using an error-prone and thermostable DNA polymerase. The fragments themselves are used as PCR primers, which align and cross-prime each other. Thus, a hybrid DNA with parts from different parent genes is obtained. Variations of DNA shuffling methods also exist where a mixture of restriction endonucleases, instead of DNasel is used, or a staggered extension process is used that does not require parental gene fragmentation. See, e.g., Antikainen and Martin, Bioorganic & Medicinal Chemistry (2005) 13:2701-2716; and Crameri et al., Nature (1998) 391:288-291.

PCR techniques, such as error-prone PCR, however, have a low error rate, i.e., approximately 0.66% (Cadwell et al., PCR Meth. Appl. (1992) 2:28-33). Random mutagenesis techniques could benefit from higher error rates in order to generate greater diversity.

Accordingly, additional methods for increasing random mutations in libraries for protein engineering purposes are highly desirable.

### SUMMARY

The present invention pertains to methods for creating randomized libraries with a high degree of diversity. The methods allow for the rapid generation of diverse protein libraries and provide for controlled, highly precise randomization within nucleic acid sequences. Such gene variant DNA libraries may be used to identify proteins with new and/or desirable functions and enable targeted modification of proteins.

In one aspect, a method for engineering a protein is provided that comprises: (a) introducing into a human lymphoblastic cell a DNA binding molecule that targets a selected protein coding region in genomic DNA present in the cell; (b) producing one or more double-strand breaks in the targeted region using a programmable endonuclease, thereby triggering DNA repair pathways to repair the breaks and produce a DNA library comprising mutated protein coding regions; and (c) screening the library to select for cells that express a protein with a trait of interest from the mutated protein coding regions, thereby providing an engineered protein. In certain embodiments, the cell is a Jurkat or CCRF-CEM cell.

In additional embodiments, a method for engineering a protein is provided that comprises: (a) introducing into a cell (i) one or more oligonucleotides that comprise about 3-50 base pairs; and (ii) a DNA binding molecule that targets a selected protein coding region in genomic DNA present in the cell; (b) producing one or more double-strand breaks in the targeted region using a programmable endonuclease, thereby triggering DNA repair pathways to repair the breaks and produce a DNA library comprising mutated protein coding regions; and (c) screening the library to select for cells that express a protein with a trait of interest from the mutated protein coding regions, thereby providing an engineered protein.

In the embodiments above, the DNA binding molecule can be a guide polynucleotide and the programmable nuclease is a Cas endonuclease. The cell can be one that constitutively expresses the Cas endonuclease or if not, the Cas endonuclease can be complexed to the guide polynucleotide prior to delivery to the cell. In certain embodiments, the DNA binding molecule is singly-guide RNA (sgRNA) and the Cas endonuclease is Cas9.

In yet a further embodiment, a method for engineering a protein is provided that comprises: (a) introducing into a human lymphoblastic cell a first DNA binding molecule that targets an integration locus region in the cell genome; (b) introducing into the cell an insertion cassette encoding a protein of interest and a selection marker, wherein the insertion cassette is flanked by 5′ and 3′ homology arms for insertion into the integration locus region in the cell; (c) producing double-strand breaks in the targeted integration locus region using a programmable endonuclease, whereby the insertion cassette is inserted into the integration locus in the cell; (d) selecting for cells comprising the coding sequence for the protein of interest; (e) introducing a second DNA binding molecule that targets the protein coding region in the selected cells; (f) producing one or more double-strand breaks in the targeted region using a second programmable endonuclease, thereby triggering DNA repair pathways to repair the breaks and produce a DNA library comprising mutated protein coding regions; and (g) screening the library to select for cells that express a protein with a trait of interest from the mutated protein coding regions, thereby providing an engineered protein. In certain embodiments, the cell is a Jurkat or CCRF-CEM cell.

In an additional embodiment, a method for engineering a protein is provided that comprises: (a) introducing into a cell (i) one or more oligonucleotides that comprise about 3-50 base pairs; and (ii) a first DNA binding molecule that targets an integration locus region in the cell genome; (b) introducing into the cell an insertion cassette encoding a protein of interest and a selection marker, wherein the insertion cassette is flanked by 5′ and 3′ homology arms for insertion into the integration locus region in the cell; (c) producing double-strand breaks in the targeted integration locus region using a programmable endonuclease, whereby the insertion cassette is inserted into the integration locus region in the cell; (d) selecting for cells comprising the coding sequence for the protein of interest; (e) introducing a second DNA binding molecule that targets the protein coding region in the selected cells; (f) producing one or more double-strand breaks in the targeted region using a second programmable endonuclease, thereby triggering DNA repair pathways to repair the breaks and produce a DNA library comprising mutated protein coding regions; and (g) screening the library to select for cells that express a protein with a trait of interest from the mutated protein coding regions, thereby providing an engineered protein.

In certain embodiments, the integration locus is Adeno-Associated Virus Integration Site 1 (AAVS1). In other embodiments, the first and/or second DNA binding molecule is a guide polynucleotide and the first and/or second programmable nuclease is a Cas endonuclease. The cell can be one that constitutively expresses the Cas endonuclease or if not, the Cas endonuclease can be complexed to the guide polynucleotide prior to delivery to the cell. In certain embodiments, the DNA binding molecule is sgRNA and the Cas endonuclease is Cas9.

In an additional embodiment, a method for preparing a protein diversification cell line is provided that comprises: (a) introducing into a cell a first DNA binding molecule that targets an integration locus region in the cell genome; (b) introducing a recombination locus cassette into the cell, wherein the recombination locus cassette is flanked by 5′ and 3′ homology arms for insertion into the integration locus region in the cell and further comprises one or more recombination acceptor sites operably linked to a promoter; (c) producing double-strand breaks in the targeted integration locus region using a programmable endonuclease, whereby the recombination locus cassette is inserted into the integration locus region in the cell; and (d) selecting for cells comprising the inserted recombination locus cassette, thereby producing a protein diversification cell line.

In certain embodiments the DNA binding molecule is a guide polynucleotide and the programmable nuclease is a Cas endonuclease. The cell can be one that constitutively expresses the Cas endonuclease or if not, the Cas endonuclease can be complexed to the guide polynucleotide prior to delivery to the cell. In certain embodiments, the DNA binding molecule is sgRNA and the Cas endonuclease is Cas9.

In additional embodiments, the integration locus is Adeno-Associated Virus Integration Site 1 (AAVS1). Additionally, the recombination acceptor sites present in the integration locus can be FRT, LoxP and AttB sites.

In further embodiments, a protein diversification cell line produced by the methods above is provided.

In additional embodiments, a method for engineering a protein is provided that comprises: (a) providing a cell from the protein diversification cell line above; (b) introducing a gene fragment library into the cell, wherein the gene fragment library comprises gene fragment sequences from a selected protein coding region; (c) introducing a recombinase expression vector into the cell, wherein the recombinase expression vector comprises one or more recombinases that drive recombination at the recombination acceptor sites in the recombination locus of the cell; whereby gene fragments from the gene fragment library are inserted into the recombinase acceptor sites to yield a mature RNA molecule in which coding exons of each gene fragment are sequentially joined in the proper order; and (d) selecting for cells that express a protein with a trait of interest from the mature RNA molecule, thereby providing an engineered protein.

In certain embodiments, the recombination acceptor sites present in the integration locus are FRT, LoxP and AttB sites and the recombinase expression vector encodes Flp, Cre and psi C31 recombinases.

In further embodiments, a method for engineering a T cell receptor (TCR) protein is provided that comprises: introducing into a human lymphoblastic cell, DNA binding molecules that target nucleotide sequences in a region present in a coding sequence for a TCR chain, wherein the region encodes complementary determining region(s) (CDR)1, CDR2 and/or CDR3;

producing one or more double-strand breaks in one or more of the coding regions for CDR1, CDR2 and/or CDR3 using a programmable endonuclease, thereby triggering DNA repair pathways to repair the breaks and produce a DNA library comprising mutated TCR coding sequences; and screening the library to select for cells that express a mutated TCR with a trait of interest, thereby providing an engineered TCR protein. In certain embodiments, the cell is a Jurkat or CCRF-CEM cell.

In additional embodiments, the TCR chains are TCRα and/or TCRβ.

In further embodiments, the DNA binding molecules target a nucleotide sequence in the regions encoding each of CDR1, CDR2 and CDR3.

In certain embodiments, the screening comprises contacting the library with an antibody that recognizes a TCR constant region and a fluorescently-tagged peptide-major histocompatibility complex (MHC), wherein the peptide represents an antigen of interest.

In certain embodiments the DNA binding molecule is a guide polynucleotide and the programmable nuclease is a Cas endonuclease. The cell can be one that constitutively expresses the Cas endonuclease or if not, the Cas endonuclease can be complexed to the guide polynucleotide prior to delivery to the cell. In certain embodiments, the DNA binding molecule is sgRNA and the Cas endonuclease is Cas9

In additional embodiments, a recombinant construct is provided. The recombinant construct comprises: a coding sequence for an enzyme capable of substituting a nucleotide base in a polynucleotide sequence; a coding sequence encoding a molecule with site-specific binding capability; and a coding sequence for a DNA repair outcome modulator. In certain embodiments, the recombinant construct is in a multicistronic configuration.

In certain embodiments, the enzyme coding sequence encodes an enzyme with deaminase activity, such as an activation-induced cytidine deaminase or an apolipoprotein B mRNA editing enzyme.

In other embodiments, the molecule coding sequence encodes a Cas endonuclease, such as Cas9, or a deactivated Cas endonuclease, such as dCas9.

In additional embodiments, the coding sequence of the modulator encodes an inhibitor of uracil DNA glycosylase or an inhibitor of the base-excision repair pathway.

In another embodiment, a method for engineering a protein with site-directed base substitution properties is provided. The method comprises: introducing into a cell a recombinant construct as described above, wherein the cell exhibits suppressed gene expression of uracil DNA glycosylase (UNG) as compared to a cell with normal UNG gene expression levels, and/or the cell overexpresses one or more components of the mismatch repair pathway (MMR), under conditions where coding sequences present in the recombinant construct are expressed to produce a DNA library comprising mutated proteins with site-directed base substitution properties; and screening the library to select for cells that express mutated proteins with site-directed base substitution activity, thereby providing an engineered protein with site-directed base substitution properties. In certain embodiments, the one or more components of MMR comprises PMS2.

These aspects and other embodiments of the methods for protein engineering will readily occur to those of ordinary skill in the art in view of the disclosure herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A and FIG. 1B present illustrative examples of Type II CRISPR-Cas9 RNAs. FIG. 1A shows a Type II CRISPR crRNA (FIG. 1A, 101) and a tracrRNA (FIG. 1A, 102), otherwise known as a dual-guide RNA. FIG. 1B illustrates the formation of base-pair hydrogen bonds between the crRNA and the tracrRNA to form secondary structure (see U.S. Published Patent Application No. 2014-0068797, published 6 Mar. 2014; see also Jinek M., et al., “A programmable dual-RNA-guided DNA endonuclease in adaptive bacterial immunity,” Science (2012) 337:816-821).

FIG. 2 shows another example of a Type II CRISPR-Cas9 associated RNA. The figure illustrates a single-guide RNA (sgRNA) wherein the crRNA is covalently joined to the tracrRNA and forms a RNA polynucleotide secondary structure through base-pair hydrogen bonding (see, e.g., U.S. Published Patent Application No. 2014-0068797, published 6 Mar. 2014). The figure presents an overview of and nomenclature for secondary structural elements of a sgRNA of the S. pyogenes Cas9.

FIG. 3A and FIG. 3B relate to structural information for a sgRNA/Cas protein complex and the domain structure of the Cas9 protein, respectively. FIG. 3A provides a model based on the crystal structure of S. pyogenes Cas9 (SpyCas9) in an active complex with sgRNA (see, e.g., Anders C., et al., “Structural basis of PAM-dependent target DNA recognition by the Cas9 endonuclease,” Nature (2014) 513:569-573). FIG. 3B presents a model of the domain arrangement of SpyCas9 relative to its primary sequence structure.

FIGS. 4A-4C depict the location of junctional diversity of the T cell receptor a (TCR α) chain (FIG. 4A); the TCR β chain (FIG. 4B); and the location of the complementary determining regions (CDRs) and variability in the TCR chains (FIG. 4C).

FIG. 5A and FIG. 5B show genotypes and phenotypes for two clonal cell lines produced in the examples: FIG. 5A, Line F3_B5; FIG. 5B, Line H3_G10.

FIGS. 6A-6F depict DNA repair outcomes at JAK1 target site (chr1:64883403-64883425 (hg38). The chromosomal locus defined here within the JAK1 gene target corresponds to the DNA target of the computationally selected protospacer sequence. An optimal target sequence is selected using parameters understood in the art. The top five repair classes and wild-type are depicted in each of FIGS. 6A-6F. DNA repair outcomes (classes and frequency) as monitored by amplicon sequencing 48 hours after nucleofection of sgRNP in HEK293 (FIG. 6A); 14 days after constitutive expression of sgRNA and Cas9 in HEK293T (FIG. 6B); 48 hours after nucleofection of sgRNP in K562 (FIG. 6C); 48 hours after nucleofection of sgRNP in donor derived T cells (FIG. 6D); 48 hours after nucleofection of sgRNP in CCRF-CEM (FIG. 6E); and 48 hours after nucleofection of sgRNP in HEK293 plus DNAPK inhibitor NU7441 (FIG. 6F). The arrows from FIG. 6A to FIGS. 6B, 6C and 6D indicate similar DNA repair outcomes compared with FIG. 6A. The arrows from FIG. 6A to FIGS. 6E and 6F indicate different DNA repair outcomes compared with FIG. 6A. Large scale computational analyses have revealed the same site-specific patterns occur across sites regardless of cell type or reagent delivery method.

FIGS. 7A and 7B depict repair outcomes at LINC00441 target site (chr13: 48303392-48303414 (hg38). The chromosomal locus defined here within the LINC00441 region corresponds to the DNA target of the computationally selected protospacer sequence. An optimal target sequence is selected using parameters understood in the art. The top fifteen repair classes and wild-type are depicted in each repair browser view. DNA repair outcomes (classes and frequency) as monitored by amplicon sequencing 48 hours after nucleofection of sgRNP in HEK293 (FIG. 7A) and Jurkat (FIG. 7B) cell lines.

FIGS. 8A-8C depict repair outcomes at BRCA1 target site (chr17:43125332-43125354 (hg38). The chromosomal locus defined here within the BRCA1 gene target corresponds to the DNA target of the computationally selected protospacer sequence. An optimal target sequence is selected using parameters understood in the art. The top fifteen repair classes and wild-type are depicted. DNA repair outcomes (classes and frequency) as monitored by amplicon sequencing 48 hours after lipofection of sgRNA (FIG. 8A); sgRNA and herring sperm DNA (200 ng) (FIG. 8B); and sgRNA and a random DNA oligo pool in a HEK293 Cas9-GFP expressing cell line (FIG. 8C).

FIGS. 9A and 9B show the Jaccard/Tanimoto coefficient for the top 10 indel repair classes (9A) and deletion-only repair classes (9B) at 96 different sites in Jurkat and HEK293 cells. The Jaccard/Tanimoto coefficient is a measure of the overlap in two sets of repairs. A value of 1 indicates complete overlap in the two sets; a value of 0 indicates no overlap in the two sets.

FIG. 10 shows the results of an experiment to determine the potential size of diverse DNA libraries created in Jurkat cells using four sgRNAs targeted to the T cell receptor beta variable 9 (TRBV9) gene.

FIG. 11 shows that the Jurkat cell repair pattern results in insertion of all 20 amino acids. The figure also illustrates the frequency of amino acid insertions using sgRNA2 targeted to TRBV9.

FIG. 12 is a schematic of three protein modules for engineering a protein with site-directed base substitution properties.

FIG. 13 is a schematic showing how three protein modules (gene fragments) can be combined using recombinase signal sequences to generate a diverse set of proteins to probe in downstream assays.

FIGS. 14A and 14B depict a representative method for engineering a Green Fluorescent Protein (GFP) in Jurkat cells. FIG. 14A shows a representative HDR cassette using an adeno-associated virus (AAV) vector; FIG. 14B shows a schematic of molecular diversity generation.

FIG. 15 shows the variable region of the β chain of the TRVB12-3 gene.

FIG. 16 is a diagram showing that the IP26 antibody recognizes an epitope in the constant region of the TCR complex while the JR.2 antibody recognizes an epitope in the variable region of the TCR β chain.

FIG. 17A and FIG. 17B show binding of IP26 and JR.2 in transfected Jurkat cells versus wild-type Jurkat cells as described in the examples. FIG. 17A shows binding of IP26 on the y-axis and JR.2 on the x-axis in transfected Jurkat cells. The cells in the trapezoid-like box are cells with decreased JR.2 binding but normal IP26 binding. FIG. 17B shows binding of JR.2 on the x-axis. The wild type Jurkat cells are in the top histogram and the transfected Jurkat cells are shown in the bottom histogram.

FIG. 18 shows that two engineered cell lines from Example 5 secreted IL-2 in response to TCR stimulation.

### DETAILED DESCRIPTION OF THE INVENTION

It is to be understood that the terminology used herein is for the purpose of describing particular embodiments only, and is not intended to be limiting. As used in this specification and the appended claims, the singular forms “a,” “an” and “the” include plural referents unless the context clearly dictates otherwise. Thus, for example, reference to “a sgRNA/Cas9 complex” includes one or more such complexes, reference to “a mutation” includes one or more mutations, and the like. It is also to be understood that when reference is made to an embodiment using a sgRNA to target Cas9 to a target site, one skilled in the art can use an alternative embodiment of the invention based on the use of a dual-guide RNA (e.g., crRNA/tracrRNA) in place of the sgRNA.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, preferred materials and methods are described herein.

In view of the teachings of the present specification, one of ordinary skill in the art can apply conventional techniques of immunology, biochemistry, chemistry, molecular biology, microbiology, cell biology, genomics, and recombinant polynucleotides, as taught, for example, by the following standard texts: Antibodies: A Laboratory Manual, Second edition, E. A. Greenfield, 2014, Cold Spring Harbor Laboratory Press, ISBN 978-1-936113-81-1; Culture of Animal Cells: A Manual of Basic Technique and Specialized Applications, 6th Edition, R. I. Freshney, 2010, Wiley-Blackwell, ISBN 978-0-470-52812-9; Transgenic Animal Technology, Third Edition: A Laboratory Handbook, 2014, C. A. Pinkert, Elsevier, ISBN 978-0124104907; The Laboratory Mouse, Second Edition, 2012, H. Hedrich, Academic Press, ISBN 978-0123820082; Manipulating the Mouse Embryo: A Laboratory Manual, 2013, R. Behringer, et al., Cold Spring Harbor Laboratory Press, ISBN 978-1936113019; PCR 2: A Practical Approach, 1995, M. J. McPherson, et al., IRL Press, ISBN 978-0199634248; Methods in Molecular Biology (Series), J. M. Walker, ISSN 1064-3745, Humana Press; RNA: A Laboratory Manual, 2010, D. C. Rio, et al., Cold Spring Harbor Laboratory Press, ISBN 978-0879698911; Methods in Enzymology (Series), Academic Press; Molecular Cloning: A Laboratory Manual (Fourth Edition), 2012, M. R. Green, et al., Cold Spring Harbor Laboratory Press, ISBN 978-1605500560; Bioconjugate Techniques, Third Edition, 2013, G. T. Hermanson, Academic Press, ISBN 978-0123822390; Methods in Plant Biochemistry and Molecular Biology, 1997, W. V. Dashek, CRC Press, ISBN 978-0849394805; Plant Cell Culture Protocols (Methods in Molecular Biology), 2012, V. M. Loyola-Vargas, et al., Humana Press, ISBN 978-1617798177; Plant Transformation Technologies, 2011, C. N. Stewart, et al., Wiley-Blackwell, ISBN 978-0813821955; Recombinant Proteins from Plants (Methods in Biotechnology), 2010, C. Cunningham, et al., Humana Press, ISBN 978-1617370212; Plant Genomics: Methods and Protocols (Methods in Molecular Biology), 2009, D. J. Somers, et al., Humana Press, ISBN 978-1588299970; Plant Biotechnology: Methods in Tissue Culture and Gene Transfer, 2008, R. Keshavachandran, et al., Orient Blackswan, ISBN 978-8173716164.

Clustered Regularly Interspaced Short Palindromic Repeats (CRISPR) and CRISPR-associated (Cas) proteins are found in prokaryotic immune systems. These systems provide resistance against exogenous genetic elements, such as viruses and plasmids, by targeting their nucleic acids for degradation, in a sequence-specific manner.

There are three main stages in CRISPR-Cas immune systems: (1) acquisition, (2) expression, and (3) interference. Acquisition involves cleaving the genome of invading viruses and plasmids and integrating segments (termed protospacers) of the genomic DNA into the CRISPR locus of the host organism. The segments that are integrated into the host genome are known as spacers, which mediate protection from subsequent attack by the same (or sufficiently related) virus or plasmid. Expression involves transcription of the CRISPR locus and subsequent enzymatic processing to produce short mature CRISPR RNAs, each containing a single spacer sequence. Interference is induced after the CRISPR RNAs associate with Cas proteins to form effector complexes, which are then targeted to complementary protospacers in foreign genetic elements to induce nucleic acid degradation.

There are several different CRISPR-Cas systems and the nomenclature and classification of these have changed as the systems have been characterized. In particular, CRISPR-Cas systems have now been reclassified into two classes, containing six types and nineteen subtypes (Makarova et al., Nature Reviews Microbiology (2015) 13:1-15; Shmakov et al., Nature Reviews Microbiology (2017) 15:169-182). This classification is based upon identifying all cas genes in a CRISPR-Cas locus and then determining the signature genes in each CRISPR-Cas locus, thereby determining whether the CRISPR-Cas systems should be placed in either Class 1 or Class 2 based upon the genes encoding the effector module, i.e., the proteins involved in the interference stage. These CRISPR-Cas systems are described in greater detail below.

A CRISPR locus includes a number of short repeating sequences referred to as “repeats.” Repeats can form hairpin structures and/or repeats can be unstructured single-stranded sequences. The repeats occur in clusters. Repeats frequently diverge between species. Repeats are regularly interspaced with unique intervening sequences, referred to as “spacers,” resulting in a repeat-spacer-repeat locus architecture. Spacers are identical to or are homologous with known foreign invader sequences. A spacer-repeat unit encodes a crisprRNA (crRNA). A crRNA refers to the mature form of the spacer-repeat unit. A crRNA contains a spacer sequence that is involved in targeting a target nucleic acid (e.g., possibly as a surveillance mechanism against foreign nucleic acid). A spacer sequence is typically located towards the 5′ end of a crRNA (e.g. in a Type I (e.g. Cascade) system; for a description of the Cascade complex see, e.g., Jore, M. M. et al., “Structural basis for CRISPR RNA-guided DNA recognition by Cascade,” Nature Structural & Molecular Biology (2011) 18:529-536) or at the 3′ end of the spacer of a crRNA in a Type II system (e.g., in a Type II CRISPR system, described more fully below), directly adjacent to the first stem.

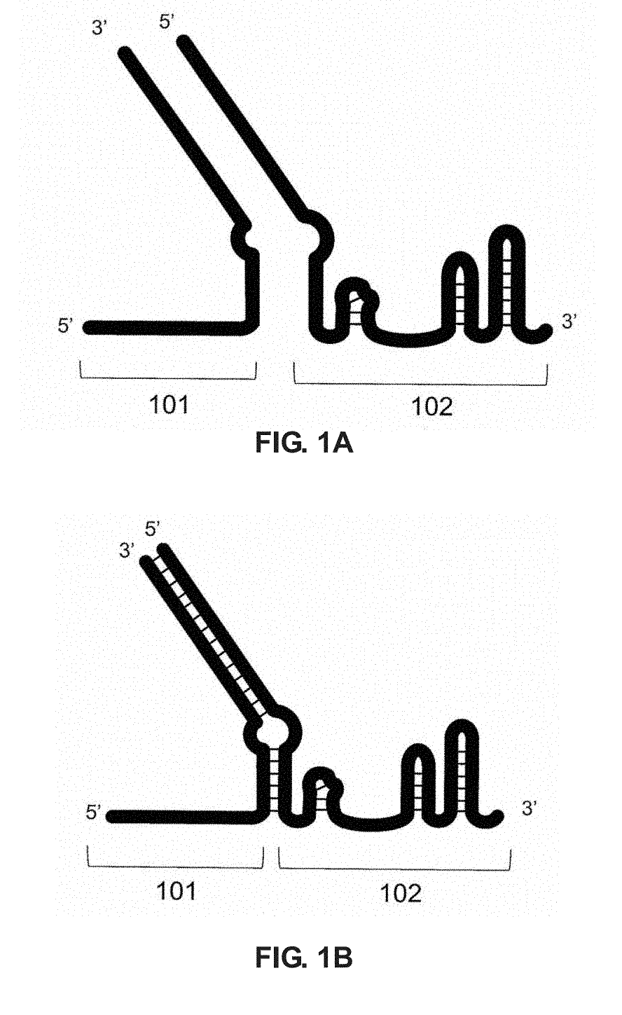

FIG. 1A and FIG. 1B present an overview of and nomenclature for secondary structural elements of the crRNA and tracrRNA of the Streptococcus pyogenes Cas9 including the following: a spacer element (FIG. 1B, 103); a first stem element comprising a lower stem element (FIG. 1B, 104), a bulge element comprising unpaired nucleotides (FIG. 1B, 105), and an upper stem element (FIG. 1B, 106); a nexus element (FIG. 1B, 107); a second hairpin element comprising a second stem element (FIG. 1B, 108); and a third hairpin element comprising a third stem element (FIG. 1B, 109). The figures are not proportionally rendered nor are they to scale. The locations of indicators are approximate.

Thus, crRNA has a region of complementarity to a potential DNA target sequence (FIG. 1A, the dark, 5′ region of the crRNA) and a second region that forms base-pair hydrogen bonds with the tracrRNA to form a secondary structure, typically to form at least a stem structure (FIG. 1A, the light region extending to the 3′ end of the crRNA). The tracrRNA and a crRNA interact through a number of base-pair hydrogen bonds to form secondary RNA structures, for example, as illustrated in FIG. 1B. Complex formation between tracrRNA/crRNA and a Cas9 protein (described more fully below) results in conformational change of the Cas protein that facilitates binding to DNA, endonuclease activities of the Cas9 protein, and crRNA-guided site-specific DNA cleavage by the endonuclease. For a Cas9 protein/tracrRNA/crRNA complex to cleave a DNA target sequence, the DNA target sequence is adjacent to a cognate protospacer adjacent motif (PAM).

A CRISPR locus comprises polynucleotide sequences encoding for CRISPR Associated (cas) genes. Cas genes are involved in the biogenesis and/or the interference stages of crRNA function. Cas genes display extreme sequence (e.g., primary sequence) divergence between species and homologues. For example, cast homologues can comprise less than 10% primary sequence identity between homologues. Some cas genes comprise homologous secondary and/or tertiary structures. For example, despite extreme sequence divergence, many members of the Cas6 family of CRISPR proteins comprise an N-terminal ferredoxin-like fold. Cas genes are named according to the organism from which they are derived. For example, cas genes in Staphylococcus epidermidis can be referred to as Csm-type, Cas genes in Streptococcus thermophilus can be referred to as Csn-type, and cas genes in Pyrococcus furiosus can be referred to as Cmr-type.

The integration stage of a CRISPR system refers to the ability of the CRISPR locus to integrate new spacers into the crRNA array upon being infected by a foreign invader. Acquisition of the foreign invader spacers can help confer immunity to subsequent attacks by the same foreign invader. Integration typically occurs at the leader end of the CRISPR locus. Cas proteins (e.g., Cas1 and Cas2) are involved in integration of new spacer sequences. Integration proceeds similarly for some types of CRISPR systems (e.g., Type I-III).

Mature crRNAs are processed from a longer polycistronic CRISPR locus transcript (i.e., pre-crRNA array). A pre-crRNA array comprises a plurality of crRNAs. The repeats in the pre-crRNA array are recognized by cas genes. Cas genes bind to the repeats and cleave the repeats. This action can liberate the plurality of crRNAs. crRNAs can be subjected to further events to produce the mature crRNA form such as trimming (e.g., with an exonuclease). A crRNA may comprise all, some, or none of the CRISPR repeat sequence.

Interference refers to the stage in the CRISPR system that is functionally responsible for combating infection by a foreign invader. CRISPR interference follows a similar mechanism to RNA interference (RNAi: e.g., wherein a target RNA is targeted (e.g., hybridized) by a short interfering RNA (siRNA)), which results in target RNA degradation and/or destabilization. CRISPR systems perform interference of a target nucleic acid by coupling crRNAs and Cas genes, thereby forming CRISPR ribonucleoproteins (crRNPs). crRNA of the crRNP guides the crRNP to foreign invader nucleic acid, (e.g., by recognizing the foreign invader nucleic acid through hybridization). Hybridized target foreign invader nucleic acid-crRNA units are subjected to cleavage by Cas proteins. Target nucleic acid interference typically requires a protospacer adjacent motif (PAM) in a target nucleic acid.

By a “CRISPR-Cas system” as used herein, is meant any of the various CRISPR-Cas classes, types and subtypes. As explained above, currently two classes of CRISPR systems have been described, Class 1 and Class 2. Class 1 systems have a multi-subunit crRNA-effector complex, whereas Class 2 systems have a single protein, such as Cas9, Cpf1, C2c1, C2c2, C2c3, or a crRNA-effector complex. Class 1 systems comprise Type I, Type III and Type IV systems. Class 2 systems comprise Type II, Type V and Type VI systems.

Class 1 systems have a multi-subunit crRNA-effector complex, whereas Class 2 systems have a single protein, such as Cas9, Cpf1, C2c1, C2c2, C2c3, or a crRNA-effector complex. Class 1 systems comprise Type I, Type III and Type IV systems. Class 2 systems comprise Type II and Type V systems.

Type I systems have a Cas3 protein that has helicase activity and cleavage activity. Type I systems are further divided into seven subtypes (I-A to I-F and I-U). Each type I subtype has a defined combination of signature genes and distinct features of operon organization. For example, subtypes I-A and I-B have the cas genes organized in two or more operons, whereas subtypes I-C through I-F appear to have the cas genes encoded by a single operon. Type I systems have a multiprotein crRNA-effector complex that is involved in the processing and interference stages of the CRISPR-Cas immune system. In E. coli, this multiprotein complex is known as CRISPR-associated complex for antiviral defense (CASCADE). Subtype I-A comprises csa5 which encodes a small subunit protein and a cas8 gene that is split into two, encoding degraded large and small subunits and also has a split cas3 gene. An example of an organism with a subtype I-A CRISPR-Cas system is Archaeoglobus fulgidus.

Subtype I-B has a cas1-cas2-cas3-cas4-cas5-cas6-cas7-cas8 gene arrangement and lacks a csa5 gene. An example of an organism with subtype I-B is Clostridium kluyveri. Subtype I-C does not have a cas6 gene. An example of an organism with subtype I-C is Bacillus halodurans. Subtype I-D has a Cas10d instead of a Cas8. An example of an organism with subtype I-D is Cyanothece sp. Subtype I-E does not have a cas4. An example of an organism with subtype I-E is Escherichia coli. Subtype I-F does not have a cas4 but has a cas2 fused to a cas3 gene. An example of an organism with subtype I-F is Yersinia pseudotuberculosis. An example of an organism with subtype I-U is Geobacter sulfurreducens.

All type III systems possess a cas10 gene, which encodes a multidomain protein containing a Palm domain (a variant of the RNA recognition motif (RRM)) that is homologous to the core domain of numerous nucleic acid polymerases and cyclases and that is the largest subunit of type III crRNA-effector complexes. All type III loci also encode the small subunit protein, one Cas5 protein and typically several Cas7 proteins. Type III can be further divided into four subtypes, III-A through III-D. Sub-type III-A has a csm2 gene encoding a small subunit and also has cas1, cas2 and cas6 genes. An example of an organism with subtype III-A is Staphylococcus epidermidis. Subtype III-B has a cmr5 gene encoding a small subunit and also typically lacks cas1, cas2 and cas6 genes. An example of an organism with subtype III-B is Pyrococcus furiosus. Subtype III-C has a Cas10 protein with an inactive cyclase-like domain and lacks a cas1 and cas2 gene. An example of an organism with subtype III-C is Methanothermobacter thermautotrophicus. Subtype III-D has a Cas10 protein that lacks the HD domain and a cast and cas2 gene, and has a cas5-like gene known as csx10. An example of an organism with subtype III-D is Roseiflexus sp.

Type IV systems encode a minimal multisubunit crRNA-effector complex comprising a partially degraded large subunit, Csf1, Cas5, Cas7, and in some cases, a putative small subunit. Type IV systems lack cast and cas2 genes. Type IV systems do not have subtypes, but there are two distinct variants. One Type IV variant has a DinG family helicase, whereas a second type IV variant lacks a DinG family helicase, but has a gene encoding a small α-helical protein. An example of an organism with a Type IV system is Acidithiobacillus ferrooxidans.

Type II systems include cas1, cas2 and cas9 genes. There are two strands of RNA in Type II systems, a CRISPR RNA (crRNA) and a transactivating CRISPR RNA (tracrRNA). The tracrRNA hybridizes to a complementary region of pre-crRNA causing maturation of the pre-crRNA to crRNA. The duplex formed by the tracrRNA and crRNA is recognized by, and associates with a multidomain protein, Cas9, encoded by the cas9 gene, that combines the functions of the crRNA-effector complex with target DNA cleavage. Cas9 is directed to a target nucleic acid by a sequence of the crRNA that is complementary to, and hybridizes with, a sequence in the target nucleic acid.

It has been demonstrated that these minimal components of the RNA-based immune system can be reprogrammed to target DNA in a site-specific manner by using a single protein and two RNA guide sequences or a single RNA molecule. Type II systems are further divided into three subtypes, subtypes II-A, II-B and II-C. Subtype II-A contains an additional gene, csn2. An example of an organism with a subtype II-A system is Streptococcus thermophilus. Subtype II-B lacks csn2, but has cas4. An example of an organism with a subtype II-B system is Legionella pneumophila. Subtype II-C is the most common Type II system found in bacteria and has only three proteins, Cas1, Cas2 and Cas9. An example of an organism with a subtype II-C system is Neisseria lactamica.

As explained above, crRNA biogenesis in a Type II CRISPR system comprises a tracrRNA. The tracrRNA is typically modified by endogenous RNaseIII. The tracrRNA hybridizes to a crRNA repeat in the pre-crRNA array. Endogenous RNaseIII is recruited to cleave the pre-crRNA. Cleaved crRNAs are subjected to exoribonuclease trimming to produce the mature crRNA form (e.g., 5′ trimming). The tracrRNA typically remains hybridized to the crRNA. The tracrRNA and the crRNA associate with a site-directed polypeptide (e.g., Cas9). The crRNA of the crRNA-tracrRNA-Cas9 complex can guide the complex to a target nucleic acid to which the crRNA can hybridize. Hybridization of the crRNA to the target nucleic acid activates a wild-type, cognate Cas9 for target nucleic acid cleavage. Target nucleic acid in a Type II CRISPR system comprises a PAM. In some embodiments, a PAM is essential to facilitate binding of a site-directed polypeptide (e.g., Cas9) to a target nucleic acid.

Cas9 is an exemplary Type II CRISPR Cas protein and serves as an endonuclease. The mature crRNA that is base-paired to trans-activating crRNA (tracrRNA) forms a two-part RNA structure, also called “dual-guide,” that directs the Cas9 to introduce double-strand breaks (DSBs) in target DNA. Cas9 can be programmed by the tracrRNA/crRNA to cleave, site-specifically, target DNA using two distinct endonuclease domains (HNH and RuvC/RNase H-like domains) (see U.S. Published Patent Application No. 2014-0068797, published 6 Mar. 2014; see also Jinek M., et al., “A programmable dual-RNA-guided DNA endonuclease in adaptive bacterial immunity,” Science (2012) 337:816-821), one for each strand of the DNA's double helix. RuvC and HNH together produce double-strand breaks, and separately can produce single-strand breaks. At sites complementary to the crRNA-guide (spacer) sequence, the Cas9 HNH nuclease domain cleaves the complementary strand and the Cas9 RuvC-like domain cleaves the non-complementary strand. Dual-crRNA/tracrRNA molecules have been engineered into single-chain crRNA/tracrRNA molecules. These single-chain crRNA/tracrRNA direct target sequence-specific Cas9 double-strand DNA cleavage.

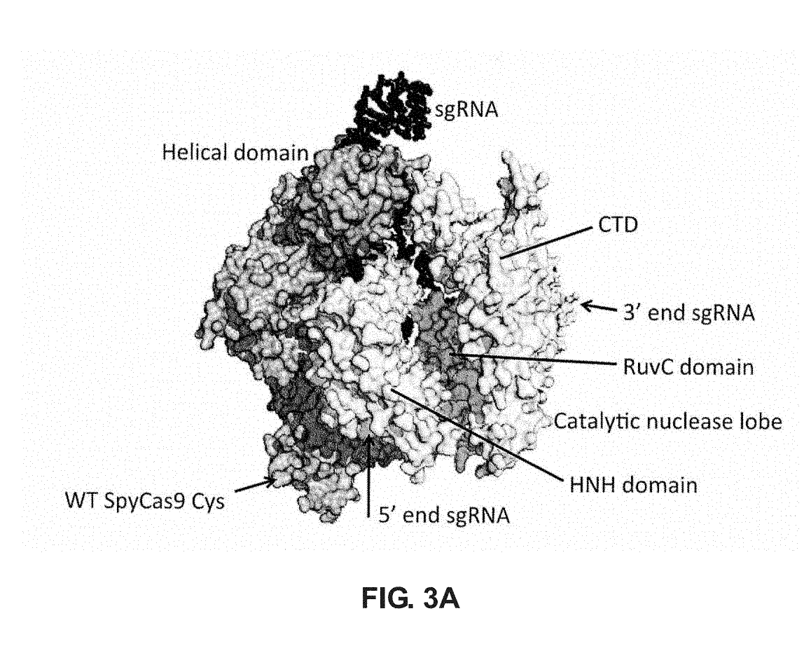

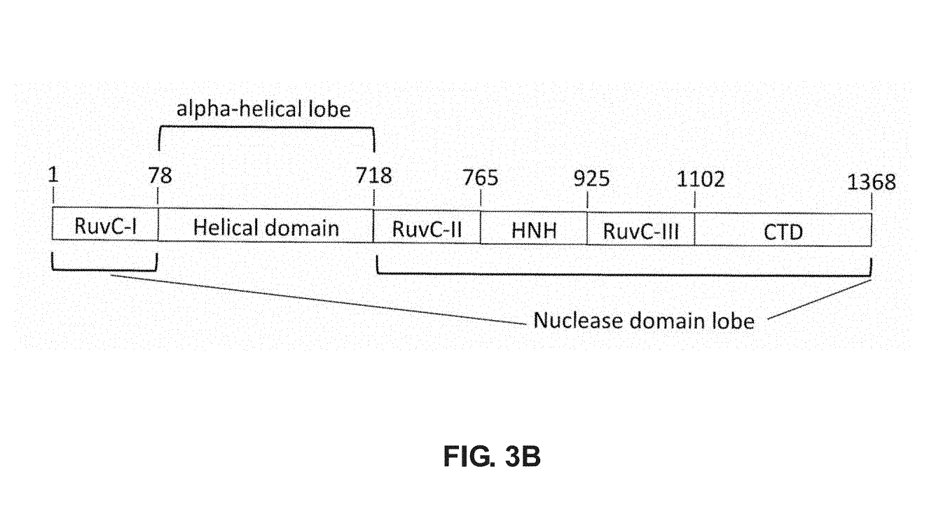

FIG. 3A presents a model of the domain arrangement of SpyCas9 (S. pyogenes Cas9) relative to its primary sequence structure and two RNA components of a Type II CRISPR-Cas9 system are illustrated in FIG. 1A. Typically, each CRISPR-Cas9 system comprises a tracrRNA and a crRNA. However, this requirement can be bypassed by using an engineered sgRNA, described more fully below, containing a designed hairpin that mimics the tracrRNA-crRNA complex (Jinek M., et al., “A programmable dual-RNA-guided DNA endonuclease in adaptive bacterial immunity,” Science (2012) 337:816-821). Base-pairing between the sgRNA and target DNA causes double-strand breaks (DSBs) due to the endonuclease activity of Cas9. Binding specificity is determined by both sgRNA-DNA base pairing and a short DNA motif (protospacer adjacent motif (PAM) sequence: NGG) juxtaposed to the DNA complementary region (Jinek et al., 2012). Thus, a Type II CRISPR system only requires a minimal set of two molecules—the Cas9 protein and the sgRNA.

A large number of Cas9 orthologs are known in the art as well as their associated tracrRNA and crRNA components (see, e.g., “Supplementary Table S2. List of bacterial strains with identified Cas9 orthologs,” Fonfara, Ines, et al., “Phylogeny of Cas9 Determines Functional Exchangeability of Dual-RNA and Cas9 among Orthologous Type II CRISPR/Cas Systems,” Nucleic Acids Research (2014) 42:2577-2590, including all Supplemental Data; Chylinski K., et al., “Classification and evolution of type II CRISPR-Cas systems,” Nucleic Acids Research (2014) 42:6091-6105, including all Supplemental Data.); Esvelt, K. M., et al., “Orthogonal Cas9 proteins for RNA-guided gene regulation and editing,” Nature Methods (2013) 10:1116-1121). A number of orthogonal Cas9 proteins have been identified including Cas9 proteins from Neisseria meningitidis, Streptococcus thermophilus and Staphylococcus aureus.

As used herein, “a Cas protein” such as “a Cas9 protein,” “a Cas3 protein,” “a Cpf1 protein,” etc. refers to a Cas protein derived from any species, subspecies or strain of bacteria that encodes the Cas protein of interest, as well as variants and orthologs of the particular Cas protein in question. The Cas proteins can either be directly isolated and purified from bacteria, or synthetically or recombinantly produced, or can be delivered using a construct encoding the protein, including without limitation, naked DNA, plasmid DNA, a viral vector and mRNA for Cas expression. Non-limiting examples of Cas proteins include Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, C2C1, C2C2, C2C3, homologs thereof, or modified versions thereof. These enzymes are known; for example, the amino acid sequence of Streptococcus pyogenes Cas9 protein may be found in the SwissProt database (available at the website uniprot.org) under accession number Q99ZW2. In some embodiments, the CRISPR protein is codon-optimized for expression in a cell of interest. In some embodiments, the CRISPR protein directs cleavage of one or two strands at the location of the target sequence. In some embodiments, the CRISPR protein lacks DNA strand cleavage activity, or acts as a nickcase. The choice of Cas protein will depend upon the particular conditions of the methods used as described herein.

Variants and modifications of Cas9 proteins are known in the art. U.S. Patent Publication 2014/0273226, published Sep. 18, 2014, incorporated herein by reference in its entirety, discusses the S. pyogenes Cas9 gene, Cas9 protein, and variants of the Cas9 protein including host-specific codon-optimized Cas9 coding sequences (e.g., ¶¶0129-0137 therein) and Cas9 fusion proteins (e.g., ¶¶233-240 therein). U.S. Patent Publication 2014/0315985, published Oct. 23, 2014, incorporated herein in its entirety, teaches a large number of exemplary wild-type Cas9 polypeptides (e.g., SEQ ID NO: 1-256, SEQ ID NOS: 795-1346, therein) including the sequence of Cas9 from S. pyogenes (SEQ ID NO: 8, therein). Modifications and variants of Cas9 proteins are also discussed (e.g., ¶¶504-608, therein). Non-limiting examples of Cas9 proteins include Cas9 proteins from S. pyogenes (GI:15675041); Listeria innocua Clip 11262 (GI:16801805); Streptococcus mutans UA159 (GI:24379809); Streptococcus thermophilus LIVID-9 (S. thermophilus A, GI:11662823; S. thermophilus B, GI:116627542); Lactobacillus buchneri NRRL B-30929 (GI:331702228); Treponema denticola ATCC 35405 (GI:42525843); Francisella novicida U112 (GI:118497352); Campylobacter jejuni subsp. Jejuni NCTC 11168 (GI:218563121); Pasteurella multocida subsp. multocida str. Pm70 (GI:218767588); Neisseria meningitidis Zs491 (GI:15602992) and Actinomyces naeslundii (GI:489880078).

The term “Cas9 protein” as used herein refers to Type II CRISPR-Cas9 proteins (as described, e.g., in Chylinski, K., (2013) “The tracrRNA and Cas9 families of type II CRISPR-Cas immunity systems,” RNA Biol. 2013 10(5):726-737), including, but not limited to Cas9, Cas9-like, proteins encoded by Cas9 orthologs, Cas9-like synthetic proteins, and variants and modifications thereof. The term as used herein refers to Cas9 wild-type proteins derived from Type II CRISPR-Cas9 systems, modifications of Cas9 proteins, variants of Cas9 proteins, Cas9 orthologs, and combinations thereof. Cas9 proteins can be derived from any of various bacterial species which genomes encode such proteins. Cas proteins for use in the present methods are described further below.

Cpf1, another CRISPR-Cas protein found in Type V systems, prefers a “TTN” PAM motif that is located 5′ to its protospacer target, not 3′, like Cas9, which recognizes a “NGG” PAM motif. Thus, Cpf1 recognizes a PAM that is not G-rich and is on the opposite side of the protospacer. Cpf1 binds a crRNA that carries the protospacer sequence for base-pairing the target. Unlike Cas9, Cpf1 does not require a separate tracrRNA and is devoid of a tracrRNA gene at the Cpf1-CRISPR locus, which means that Cpf1 only requires a crRNA that is about 43 bases long. 24 nt represents the protospacer and 19 nt the constitutive direct repeat sequence. Cpf1 appears to be directly responsible for cleaving the 43 base crRNAs apart from the primary transcript (Fonfara et al., (2016) “The CRISPR-associated DNA-cleaving enzyme Cpf1 also processes precursor CRISPR RNA,” Nature 532:517-521).

Aspects of the present invention can be practiced by one of ordinary skill in the art following the guidance of the specification to use CRISPR-Cas proteins, such as CRISPR-Cas9, Cas3, Cpf1 proteins and Cas-protein encoding polynucleotides, including, but not limited to proteins encoded by the native sequences and proteins encoded by Cas orthologs, Cas-like synthetic proteins, and variants and modifications thereof. The cognate RNA components of these Cas proteins can be manipulated and modified for use in the practice of the present invention by one of ordinary skill in the art following the guidance of the present specification.

A “nucleic acid-targeting nucleic acid” (NATNA), also known as a “guide polynucleotide” refers to one or more polynucleotides that guide a protein, such as a Cas9, Cas3, etc., protein, or a deactivated Cas endonuclease, to preferentially target a nucleic acid target sequence present in a polynucleotide (relative to a polynucleotide that does not comprise the nucleic acid target sequence). NATNAs can comprise ribonucleotide bases (e.g., RNA), deoxyribonucleotide bases (e.g., DNA), combinations of ribonucleotide bases and deoxyribonucleotide bases (e.g., RNA/DNA), nucleotides, nucleotide analogs, modified nucleotides, and the like, as well as synthetic, naturally occurring, and non-naturally occurring modified backbone residues or linkages. Thus, a NATNA as used herein site-specifically guides a Cas9, Cas3, etc. to a target nucleic acid. Many such NATNAs are known, such as but not limited to sgRNA (including miniature and truncated single-guide RNAs), crRNA, dual-guide RNA, including but not limited to, crRNA/tracrRNA molecules, as described herein, and the like, the use of which depends on the particular Cas protein. For a non-limiting description of exemplary NATNAs, see, e.g., PCT Publication No. WO 2014/150624 to May et al., published Sep. 29, 2014; PCT Publication No. WO 2015/200555 to May et al., published Mar. 10, 2016; PCT Publication No. WO 2016/201155 to Donohoue et al., published Dec. 15, 2016; PCT Publication No. WO 2017/027423 to Donohoue et al., published Feb. 16, 2017; and PCT Publication No. WO 2016/123230 to May et al., published Aug. 4, 2016; each of which is incorporated herein by reference in its entirety. The terms “NATNA” and “guide polynucleotide” as used herein also intend a Zinc finger DNA-binding domain, a Transcription activator-like (TAL) effector DNA binding domain, and the like, that guide a non-CRISPR endonuclease cleavage domain to a selected site.

As used herein, a Cas protein (e.g., a Cas9 protein) is said to “target” a polynucleotide if a NATNA/Cas protein nucleoprotein complex associates with, binds and/or cleaves a polynucleotide at the nucleic acid target sequence within the polynucleotide.

With reference to a NATNA, a “spacer” or “spacer element” as used herein, refers to the polynucleotide sequence that can specifically hybridize to a target nucleic acid sequence. The spacer element interacts with the target nucleic acid sequence through hydrogen bonding between complementary base pairs (i.e., paired bases). A spacer element binds to a selected nucleic acid target sequence. Accordingly, the spacer element is the nucleic acid target-binding sequence. The spacer element determines the location of a Cas protein's site-specific binding and nucleolytic cleavage. Spacer elements range from approximately 17 to approximately 84 nucleotides in length and have an average length of 36 nucleotides (see, e.g., Marraffini, et al., “CRISPR interference: RNA-directed adaptive immunity in bacteria and archaea,” Nature reviews Genetics (2010) 11:181-190). For example, for SpyCas9, the functional length for a spacer to direct specific cleavage is typically about 12-25 nucleotides. Variability of the functional length for a spacer element is known in the art (e.g., U.S. Published Patent Application No. 20140315985, published 23 Oct. 2014, incorporated herein by reference in its entirety). The terms “nucleic acid target binding sequence” and “spacer sequence” are used interchangeably herein.

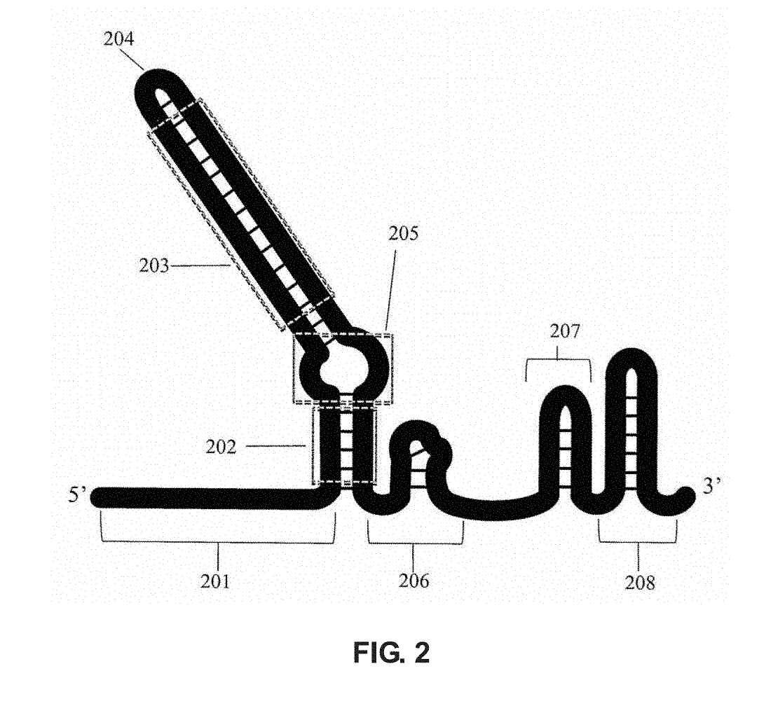

The term “sgRNA” typically refers to a single-guide RNA (i.e., a single, contiguous polynucleotide sequence) that essentially comprises a crRNA connected at its 3′ end to the 5′ end of a tracrRNA through a “loop” sequence (see, e.g., U.S. Published Patent Application No. 2014/0068797 to Doudna et al., published 6 Mar. 2014 and incorporated herein by reference in its entirety). sgRNA interacts with a cognate Cas protein essentially as described for tracrRNA/crRNA polynucleotides. Similar to crRNA, sgRNA has a spacer, a region of complementarity to a potential DNA target sequence (FIG. 2, 201), adjacent a second region that forms base-pair hydrogen bonds that form a secondary structure, typically a stem structure (FIG. 2, 202, 203, 204, 205). The term includes truncated single-guide RNAs (tru-sgRNAs) of approximately 17-18 nucleotides (nt) (see, e.g., Fu et. al., “Improving CRISPR-Cas nuclease specificity using truncated guide RNAs,” Nat Biotechnol. (2014) 32:279-284). The term also encompasses functional miniature sgRNAs with expendable features removed, but that retain an essential and conserved module termed the “nexus” located in the portion of sgRNA that corresponds to tracrRNA (not crRNA). See, e.g., U.S. Patent Publication 2014/0315985 to May et al., published Oct. 23, 2014, incorporated herein by reference in its entirety; Briner et al., “Guide RNA Functional Modules Direct Cas9 Activity and Orthogonality,” Molecular Cell (2014) 56:333-339. The nexus is located immediately downstream of (i.e., located in the 3′ direction from) the lower stem in Type II CRISPR-Cas9 systems. An example of the relative location of the nexus is illustrated in the sgRNA shown in FIG. 2. The nexus confers the binding of a sgRNA or a tracrRNA to its cognate Cas9 protein and confers an apoenzyme to haloenzyme conformational transition.

FIG. 3A provides a three-dimensional model based on the crystal structure of S. pyogenes Cas9 (SpyCas9) in an active complex with sgRNA. Structural studies of the SpyCas9 show that the protein exhibits a bi-lobed architecture comprising the Catalytic nuclease lobe and the α-Helical lobe of the enzyme (See Jinek M., et al., “Structures of Cas9 endonucleases reveal RNA-mediated conformational activation,” Science (2014) 343:1247997; Anders C., et al., “Structural basis of PAM-dependent target DNA recognition by the Cas9 endonuclease,” Nature (2014) 513:569-573).

The relationship of the sgRNA to the Helical domain and the Catalytic domain is illustrated in FIG. 3A. The 3′ and 5′ ends of the sgRNA are indicated, as well as exposed portions of the sgRNA. The spacer RNA of the sgRNA is not visible because it is surrounded by the α-Helical lobe (Helical domain) and the Catalytic nuclease lobe (Catalytic domain). The spacer RNA of the sgRNA is located in the 5′ end region of the sgRNA. The RuvC and HNH nuclease domains, when active, each cut a different DNA strand in target DNA. The C-terminal domain (CTD) is involved in recognition of protospacer adjacent motifs (PAMs) in target DNA.

In FIG. 3A, the α-Helical lobe (FIG. 3A, Helical domain) is shown as the darker lobe; the Catalytic nuclease lobe (FIG. 3A, Catalytic nuclease lobe) is shown in a light grey and the sgRNA is shown in black (FIG. 3A, sgRNA). A cysteine residue (FIG. 3A, WT SpyCas9 Cys) in wild-type SpyCas9 is identified in the present disclosure as an available cross-linking site. In FIG. 3A, the Catalytic nuclease lobe is shown as the lighter lobe wherein the relative positions of the RuvC (FIG. 3A, RuvC; RNase H homologous domain) and HNH nuclease (FIG. 3A, HNH; HNH nuclease homologous domain) domains are indicated. The RuvC and HNH nuclease domains, when active, each cut a different DNA strand in target DNA. The C-terminal domain (FIG. 3A, CTD) is involved in recognition of protospacer adjacent motifs (PAM) in target DNA.

FIG. 3B presents a model of the domain arrangement of SpyCas9 relative to its primary sequence structure. In FIG. 3B, three regions of the primary sequence correspond to the RuvC domain (FIG. 3B, RuvC-I (amino acids 1-78), RuvC-II (amino acids 719-765), and RuvC-III (amino acids 926-1102)). One region corresponds to the Helical domain (FIG. 3B, Helical Domain (amino acids 79-718). One region corresponds to the HNH domain (FIG. 3B, HNH (amino acids 766-925). One region corresponds to the CTD domain (FIG. 3B, CTD (amino acids 1103-1368). In FIG. 3B, the regions of the primary sequence corresponding to the α-Helical lobe (FIG. 3B, alpha-helical lobe) and the Nuclease domain lobe (FIG. 3B, Nuclease domain lobe) are indicated with brackets.

U.S. Patent Publication No. 2014/0315985, published 23 Oct. 2014, incorporated herein by reference in its entirety; and Briner et al., “Guide RNA Functional Modules Direct Cas9 Activity and Orthogonality,” Molecular Cell (2014) 56:333-339, disclose consensus sequences and secondary structures of predicted sgRNAs for several sgRNA/Cas9 families. The general arrangement of secondary structures in the predicted sgRNAs up to and including the nexus are presented in FIG. 2 herein which presents an overview of and nomenclature for elements of the sgRNA of the S. pyogenes Cas9 including the following: a spacer element (FIG. 2, 201); a first stem element comprising a lower stem element (FIG. 2, 202), a bulge element comprising unpaired nucleotides (FIG. 2, 205), and an upper stem element (FIG. 2, 203); a loop element (FIG. 2, 204) comprising unpaired nucleotides; (a first hairpin element comprises the first stem element and the loop element); a nexus element (FIG. 2, 206); a second hairpin element comprising a second stem element (FIG. 2, 207); and a third hairpin element comprising a third stem element (FIG. 2, 208). (See, e.g., FIGS. 1 and 3 of Briner, A. E., et al., “Guide RNA Functional Modules Direct Cas9 Activity and Orthogonality,” Molecular Cell (2014) 56:333-339.) The figure is not proportionally rendered nor is it to scale. The locations of indicators are approximate.

Relative to FIG. 2, there is variation in the number and arrangement of stem structures located 3′ of the nexus in the sgRNAs of U.S. Published Patent Application No. 2014-0315985 and Briner, et al.

Ran et al., “In vivo genome editing using Staphylococcus aureus Cas9,” Nature (2015) 520:186-191, including all extended data) present the crRNA/tracrRNA sequences and secondary structures of eight Type II CRISPR-Cas9 systems (see Extended Data FIG. 1 of Ran, et al.). Further, Fonfara, et al., (“Phylogeny of Cas9 Determines Functional Exchangeability of Dual-RNA and Cas9 among Orthologous Type II CRISPR/Cas Systems,” Nucleic Acids Research (2014) 42:2577-2590, including all Supplemental Data, in particular Supplemental Figure S11) present the crRNA/tracrRNA sequences and secondary structures of eight Type II CRISPR-Cas9 systems.

As used herein, “dual-guide RNA” refers to a two-component RNA system for a polynucleotide component capable of associating with a cognate Cas protein. A representative CRISPR Class 2 Type II CRISPR-Cas-associated dual-guide RNA includes a Cas-crRNA and Cas-tracrRNA, paired by hydrogen bonds to form secondary structure (see, e.g., U.S. Published Patent Application No. 2014/0068797 to Doudna et al., published 6 Mar. 2014 and incorporated herein by reference in its entirety; see also Jinek M., et al., Science 337:816-21 (2012)). A Cas-dual-guide RNA is capable of forming a nucleoprotein complex with a cognate Cas protein, wherein the complex is capable of targeting a nucleic acid target sequence complementary to the spacer sequence.

As used herein, the term “cognate” typically refers to a Cas protein (e.g., Cas9 protein) and one or more polynucleotides (e.g., a CRISPR-Cas9-associated NATNA) that are capable of forming a nucleoprotein complex capable of site-directed binding to a nucleic acid target sequence complementary to the nucleic acid target binding sequence present in one of the one or more polynucleotides.

By “donor polynucleotide” is meant a polynucleotide that can be directed to, and inserted into a target site of interest, such as an integration locus, to modify the target nucleic acid. All or a portion of the donor polynucleotide can be inserted into the target nucleic acid. The donor polynucleotide can be used for repair of the break in the target DNA sequence resulting in the transfer of genetic information (i.e., polynucleotide sequences) from the donor at the site or in close proximity of the break in the DNA. Accordingly, new genetic information (i.e., polynucleotide sequences) may be inserted or copied at a target DNA site. The donor polynucleotide can be double- or single-stranded DNA, RNA, a vector, plasmid, or the like. Thus, a donor polynucleotide can be an insertion cassette, a recombinase expression vector, and the like, as described further below. Non-symmetrical polynucleotide donors can also be used that are composed of two DNA oligonucleotides. They are partially complementary, and each can include a flanking region of homology. The donor can be used to insert or replace polynucleotide sequences in a target sequence, for example, to introduce a polynucleotide that encodes a protein or functional RNA (e.g., siRNA), to introduce a protein tag, to modify a regulatory sequence of a gene, or to introduce a regulatory sequence to a gene (e.g. a promoter, an enhancer, an internal ribosome entry sequence, a start codon, a stop codon, a localization signal, or polyadenylation signal), to modify a nucleic acid sequence (e.g., introduce a mutation), and the like.

Targeted DNA modifications using donor polynucleotides for large changes (e.g., more than 100 bp insertions or deletions) traditionally use plasmid-based donor templates that contain homology arms flanking the site of alteration. Each arm can vary in length, but is typically longer than about 100 bp, such as 100-1500 bp, e.g., 100 . . . 200 . . . 300 . . . 400 . . . 500 . . . 600 . . . 700 . . . 800 . . . 900 . . . 1000 . . . 1500 bp or any integer between these values. However, these numbers can vary, depending on the size of the donor polynucleotide and the target polynucleotide. This method can be used to generate large modifications, including insertion of reporter genes such as fluorescent proteins or antibiotic resistance markers. For transfection in cells, such as HEK cells, approximately 100-1000 ng, e.g., 100 . . . 200 . . . 300 . . . 400 . . . 500 . . . 600 . . . 700 . . . 800 . . . 900 . . . 1000 ng or any integer between these values, of a typical size donor plasmid (e.g., approximately 5 kb) containing a sgRNA/Cas9 vector, can be used for one well in 24-well plate. (See, e.g., Yang et al., “One Step Generation of Mice Carrying Reporter and Conditional Alleles by CRISPR/Cas-Mediated Genome Engineering” Cell (2013) 154:1370-1379).

Single-stranded and partially double-stranded oligonucleotides, such as DNA oligonucleotides, have been used in place of targeting plasmids for short modifications (e.g., less than 50 bp) within a defined locus without cloning. To achieve high HDR efficiencies, single-stranded oligonucleotides containing flanking sequences on each side that are homologous to the target region can be used, and can be oriented in either the sense or antisense direction relative to the target locus. The length of each arm can vary in length, but the length of at least one arm is typically longer than about 10 bases, such as from 10-150 bases, e.g., 10 . . . 20 . . . 30 . . . 40 . . . 50 . . . 60 . . . 70 . . . 80 . . . 90 . . . 100 . . . 110 . . . 120 . . . 130 . . . 140 . . . 150, or any integer within these ranges. However, these numbers can vary, depending on the size of the donor polynucleotide and the target polynucleotide. In a preferred embodiment, the length of at least one arm is 10 bases or more. In other embodiments, the length of at least one arm is 20 bases or more. In yet other embodiments, the length of at least one arm is 30 bases or more. In some embodiments, the length of at least one arm is less than 100 bases. In further embodiments, the length of at least one arm is greater than 100 bases. In some embodiments, the length of at least one arm is zero bases. For single-stranded DNA oligonucleotide design, typically an oligonucleotide with around 100-150 bp total homology is used. The mutation is introduced in the middle, giving 50-75 bp homology arms for a donor designed to be symmetrical about the target site. In other cases, no homology arms are required, and the donor polynucleotide is inserted using non-homologous DNA repair mechanisms.

A “genomic region” is a segment of a chromosome in the genome of a host cell that is present on either side of the nucleic acid target sequence site or, alternatively, also includes a portion of the nucleic acid target sequence site. The homology arms of the donor polynucleotide have sufficient homology to undergo homologous recombination with the corresponding genomic regions. In some embodiments, the homology arms of the donor polynucleotide share significant sequence homology to the genomic region immediately flanking the nucleic acid target sequence site; it is recognized that the homology arms can be designed to have sufficient homology to genomic regions farther from the nucleic acid target sequence site.

The terms “wild-type,” “naturally occurring” and “unmodified” are used herein to mean the typical (or most common) form, appearance, phenotype, or strain existing in nature; for example, the typical form of cells, organisms, characteristics, polynucleotides, proteins, macromolecular complexes, genes, RNAs, DNAs, or genomes as they occur in and can be isolated from a source in nature. The wild-type form, appearance, phenotype, or strain serve as the original parent before an intentional modification. Thus, mutant, variant, chimeric, engineered, recombinant, and modified forms are not wild-type forms.

As used herein, the terms “engineered,” “genetically engineered,” “recombinant,” “modified,” and “non-naturally occurring” are interchangeable and indicate intentional human manipulation.

As used herein, the terms “nucleic acid,” “nucleotide sequence,” “oligonucleotide,” and “polynucleotide” are interchangeable. All refer to a polymeric form of nucleotides. The nucleotides may be deoxyribonucleotides (DNA) or ribonucleotides (RNA), or analogs thereof, and they may be of any length. Polynucleotides may perform any function and may have any secondary structure and three-dimensional structure. The terms encompass known analogs of natural nucleotides and nucleotides that are modified in the base, sugar and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include methylated nucleotides and nucleotide analogs. Nucleotide structure may be modified before or after a polymer is assembled. Following polymerization, polynucleotides may be additionally modified via, for example, conjugation with a labeling component or target-binding component. A nucleotide sequence may incorporate non-nucleotide components. The terms also encompass nucleic acids comprising modified backbone residues or linkages, that (i) are synthetic, naturally occurring, and non-naturally occurring, and (ii) have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), and morpholino structures.

Polynucleotide sequences are displayed herein in the conventional 5′ to 3′ orientation.

As used herein, the term “complementarity” refers to the ability of a nucleic acid sequence to form hydrogen bond(s) with another nucleic acid sequence (e.g., through traditional Watson-Crick base pairing). A percent complementarity indicates the percentage of residues in a nucleic acid molecule that can form hydrogen bonds with a second nucleic acid sequence. When two polynucleotide sequences have 100% complementarity, the two sequences are perfectly complementary, i.e., all of a first polynucleotide's contiguous residues hydrogen bond with the same number of contiguous residues in a second polynucleotide.

As used herein, the term “sequence identity” generally refers to the percent identity of bases or amino acids determined by comparing a first polynucleotide or polypeptide to a second polynucleotide or polypeptide using algorithms having various weighting parameters. Sequence identity between two polypeptides or two polynucleotides can be determined using sequence alignment by various methods and computer programs (e.g., BLAST, CS-BLAST, FASTA, HMMER, L-ALIGN, etc.), available through the worldwide web at sites including GENBANK (ncbi.nlm.nih.gov/genbank/) and EMBL-EBI (ebi.ac.uk.). Sequence identity between two polynucleotides or two polypeptide sequences is generally calculated using the standard default parameters of the various methods or computer programs. Generally, the Cas proteins for use herein will have at least about 75% or more sequence identity to the wild-type or naturally occurring sequence of the Cas protein of interest, such as about 80%, such as about 85%, 90%, 91%, 92%, 93%, 94%, 95%, 96%, 97%, 98%, 99%, or complete identity.

As used herein a “stem-loop structure” or “stem-loop element” refers to a polynucleotide having a secondary structure that includes a region of nucleotides that are known or predicted to form a double-stranded region (the “stem element”) that is linked on one side by a region of predominantly single-stranded nucleotides (the “loop element”). The term “hairpin” element is also used herein to refer to stem-loop structures. Such structures are well known in the art. The base pairing may be exact. However, as is known in the art, a stem element does not require exact base pairing. Thus, the stem element may include one or more base mismatches or non-paired bases.

As used herein, “double-strand break” (DSB) refers to both strands of a double-stranded segment of nucleic acid being severed. In some instances, if such a break occurs, one strand can be said to have a “sticky end” wherein nucleotides are exposed and not hydrogen bonded to nucleotides on the other strand. In other instances, a “blunt end” can occur wherein both strands remain fully base paired with each other.

As used herein, the term “recombination” refers to a process of exchange of genetic information between two polynucleotides.

As used herein, “nucleic acid repair,” such as but not limited to DNA repair, encompasses any process whereby cellular machinery repairs damage to a nucleic acid molecule contained in the cell. The damage repaired can include single-strand breaks, double-strand breaks (DSBs), or mis-incorporation of bases.

As used herein, DNA mismatch repair (MMR) refers to a system for recognizing and repairing erroneous insertion, deletion, and/or mis-incorporation of nucleic acid bases that can arise, e.g., during DNA replication and recombination. Examples of mismatched bases include, for example, a G/T or A/C pairing, as opposed to the proper G/C or A/T pairing. Damage is repaired by recognizing the deformity caused by the mismatch, determining the template and non-template strand, excising the wrongly incorporated base, and replacing it with the correct nucleotide. Mismatch repair is strand-specific. In order to begin repair, the mismatch repair machinery distinguishes the newly synthesized daughter strand from the template parental strand.

As used herein “base-excision repair” (BER) refers to a cellular mechanism that repairs damaged DNA throughout the cell cycle. It is responsible primarily for removing small, non-helix-distorting base lesions from the genome. BER is initiated by DNA glycosylases, which recognize and remove specific damaged or inappropriate bases, forming AP sites. These are then cleaved by an AP endonuclease. The resulting single-strand break can then be processed by either short-patch (where a single nucleotide is replaced) or long-patch BER (where 2-10 new nucleotides are synthesized. More particularly, the BER pathway involves five key enzymatic steps to remove the initial DNA lesion and restore the genetic material back to its original state: (i) excision of a damaged or inappropriate base, (ii) incision of the phosphodiester backbone at the resulting abasic site, (iii) termini clean-up to permit unabated repair synthesis and/or nick ligation, (iv) gap-filling to replace the excised nucleotide, and (v) sealing of the final, remaining DNA nick. These repair steps are executed by a collection of enzymes that include DNA glycosylases, apurinic/apyrimidinic endonucleases, phosphatases, phosphodiesterases, kinases, polymerases and ligases. For a review of BER, see, e.g., Seeburg et al., Trends in Biochem. Sci. (1995) 20:391-397; Kim et al., Curr. Mol. Pharmacol. (2012) 5:3-13.

As used herein, the term “homology-directed repair” or “HDR” refers to DNA repair that takes place in cells, for example, during repair of double-strand and single-strand breaks in DNA. HDR requires nucleotide sequence homology and uses a “donor template” (donor template DNA, polynucleotide donor, or oligonucleotide (used interchangeably herein) to repair the sequence where the double-strand break occurred (e.g., DNA target sequence). This results in the transfer of genetic information from, for example, the donor template DNA to the DNA target sequence. HDR may result in alteration of the DNA target sequence (e.g., insertion, deletion, mutation) if the donor template DNA sequence or oligonucleotide sequence differs from the DNA target sequence and part or all of the donor template DNA polynucleotide or oligonucleotide is incorporated into the DNA target sequence. In some embodiments, an entire donor template DNA polynucleotide, a portion of the donor template DNA polynucleotide, or a copy of the donor polynucleotide is copied or integrated at the site of the DNA target sequence.

As used herein the terms “classical non-homologous end joining” or “c-NHEJ” refer to the repair of double-strand breaks in DNA by direct ligation of one end of the break to the other end of the break without a requirement for a donor template DNA. NHEJ in the absence of a donor template DNA often results in small insertions or deletions of nucleotides at the site of the double-strand break, also referred to as “indels.” This DNA repair pathway is genetically defined and requires the activity of Ligase IV, DNA-PKcs, Polμ, Polλ, and the Ku70/80 heterodimer, among other proteins (Sfeir and Symington, Trends Biochem Sci (2015) 40:701-714).

“Microhomology-mediated end joining (MMEJ),” a form of alternative nonhomologous end-joining (alt-NHEJ) is another pathway for repairing double-strand breaks in DNA. MMEJ is associated with deletions flanking a DSB and involves alignment of microhomologous sequences internal to the broken ends before joining. The proposed mechanism entails 5′ to 3′ resection of the DNA ends at a DSB, annealing of the microhomologies (1-16 nucleotides of homology), removal of heterologous flaps, gap filling DNA synthesis, and ligation. MMEJ is genetically defined and requires the activity of CtIP, PARP1, Polθ, Lig1 and Lig3, among other proteins (Sfeir and Symington, Trends Biochem Sci (2015) 40:701-714).

Alternative mechanisms of DNA insertion that do not require sequence homology between the donor and the target sequence can also be used for nucleic acid insertion. These mechanisms involve various components of the cellular DNA repair machinery and it is to be understood that the scope of the invention is not bound by the use of any particular mechanism for insertion of nucleic acid after target nucleic acid is cut or nicked by a site-specific polynucleotide.

The terms “vector” and “plasmid” are used interchangeably and as used herein refer to a polynucleotide vehicle to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, viral vectors, cosmids, and artificial chromosomes. As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein the term “expression cassette” is a polynucleotide construct, generated recombinantly or synthetically, comprising regulatory sequences operably linked to a selected polynucleotide to facilitate expression of the selected polynucleotide in a host cell.

For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like.

As used herein the term “operably linked” refers to polynucleotide sequences or amino acid sequences placed into a functional relationship with one another. For instance, a promoter or enhancer is operably linked to a coding sequence if it regulates, or contributes to the modulation of, the transcription of the coding sequence. Operably linked DNA sequences encoding regulatory sequences are typically contiguous to the coding sequence. However, enhancers can function when separated from a promoter by up to several kilobases or more. Additionally, multicistronic constructs can include multiple coding sequences which use only one promoter by including a 2A self-cleaving peptide, an IRES element, etc. Accordingly, some polynucleotide elements may be operably linked but not contiguous.

As used herein, the term “expression” refers to transcription of a polynucleotide from a DNA template, resulting in, for example, an mRNA or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product.” Expression may include splicing the mRNA in a eukaryotic cell, if the polynucleotide is derived from genomic DNA.

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms may be used to refer to an amino acid polymer that has been modified through, for example, acetylation, disulfide bond formation, glycosylation, lipidation, phosphorylation, cross-linking, and/or conjugation (e.g., with a labeling component or ligand). Polypeptide sequences are displayed herein in the conventional N-terminal to C-terminal orientation.

Polypeptides and polynucleotides can be made using routine techniques in the field of molecular biology (see, e.g., standard texts discussed above). Further, essentially any polypeptide or polynucleotide can be custom ordered from commercial sources.

The term “binding” as used herein includes a non-covalent interaction between macromolecules (e.g., between a protein and a polynucleotide, between a polynucleotide and a polynucleotide, and between a protein and a protein). Such non-covalent interaction is also referred to as “associating” or “interacting” (e.g., when a first macromolecule interacts with a second macromolecule, the first macromolecule binds to second macromolecule in a non-covalent manner). Some portions of a binding interaction may be sequence-specific; however, all components of a binding interaction do not need to be sequence-specific, such as a protein's contacts with phosphate residues in a DNA backbone. Binding interactions can be characterized by a dissociation constant (Kd). “Affinity” refers to the strength of binding. An increased binding affinity is correlated with a lower Kd. An example of non-covalent binding is hydrogen bond formation between base pairs.

As used herein, the term “isolated” can refer to a nucleic acid or polypeptide that, by the hand of a human, exists apart from its native environment and is therefore not a product of nature. Isolated means substantially pure. An isolated nucleic acid or polypeptide can exist in a purified form and/or can exist in a non-native environment such as, for example, in a recombinant cell.

As used herein, a “host cell” generally refers to a biological cell. A cell can be the basic structural, functional and/or biological unit of a living organism. A cell can originate from any organism having one or more cells. Examples of host cells include, but are not limited to: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, sunflower, sorghum, millet, alfalfa, oil-producing Brassica (for example, but not limited to, oilseed rape/canola), pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.). Further, a cell can be a stem cell or progenitor cell.

### Protein Engineering Techniques

The present invention is directed to the efficient and rapid production of highly diverse molecular libraries for identifying proteins with new and/or altered functions, e.g., enhanced or decreased activity. For example, these mutant libraries can be screened for new proteins that show enhanced expression levels, solubility, stability, enzymatic activity, and/or interaction with desired binding partners. Such engineered proteins are important as therapeutics, diagnostics, and imaging agents in biological systems.

Typically, the methods described herein make use of programmable endonucleases, i.e., proteins that recognize specific nucleotide sequences and that are capable of introducing double-strand breaks within these sequences. In this way, diversity can be introduced in libraries, and/or specific domains predicted to have a role in protein function can be targeted. Additionally, multiplexed targeting of mutations can be accomplished, using multiple programmable nucleases targeted to the same or different domains, thus allowing for the introduction of diversity at multiple sites simultaneously.

Programmable endonucleases for use in the present methods include, without limitation, those from the CRISPR-Cas systems, Zinc-finger nucleases (ZFNs), Transcription activator-like effector nucleases (TALENs), meganucleases, MEGA-TALs, Argonaute (Ago), and others known to one of skill in the art. See, e.g., Gao et al., Nature Biotechnology (2016) 34:768-773. These endonucleases can be used to generate libraries for any protein for the purpose of identifying a mutant with a new or altered function.

Using these methods, any protein coding sequence can be targeted. Particular known proteins for protein engineering by the methods described herein include, but are not limited to, mammalian antibodies (ABs) (IgG, IgA, IgM, IgE), antibody fragments such as Fc regions, antibody Fab regions, antibody heavy chains, antibody light chains, antibody CDRs, nanobodies, chimeric antibodies and other IgG domains; T cell receptors (TCRs); endonucleases and exonucleases, such as TALENs, CRISPR nucleases such as Cas9, Cas3, Cpf1, ZFNs, meganucleases, nuclease domains such as HNH domain, RuvC domain; recombinases such as Cre, Tre, Brec1, Flp, γ-integrase, IntI4 integrase, XerD recombinase, HP1 integrase; DNA topoisomerases; transposons such as the Tc1/mariner family, Tol2, piggyBac, Sleeping beauty; RAG proteins; retrotransposons such as LTR-retrotransposons and non-LTR retrotransposons (Alu, SINE, LINE); enzymes including but not limited to arginases, glycosydases, proteases, kinases, and glycosylation enzymes such as glycosyltransferase; anticoagulants such as protein C, Protein S and antithrombin; coagulants such as thrombin; nucleases such as DNAses, RNAses, helicases, GTPases; DNA or RNA binding proteins; reporter molecules, such as Green Fluorescent Protein (GFP); cell penetrating peptides and their fusions with cargo proteins; membrane proteins such as GPCRs, pain receptors such as TRP channels and ion channels; cell surface receptors including but not limited to EGFR, FGFR, VEGFR, IGFR and ephrin receptor; cell adhesion molecules like integrins and cadherins; ion channels; rhodopsins; immunoreceptors such as CD28, CD80, PD-1, PD-L1, CTLA-4, CXCR4, CXCR5, B2M, TRACA, TRBC; secreted proteins including but not limited to hormones, cytokines, growth factors; vaccine antigens such as viral proteins from human immunodeficiency virus (HIV), Dengue, cytomegalovirus (CMV), Ebola, Zika and oncolytic viruses; snake toxin proteins and peptides including but not limited to phospholipases and metalloproteases; ribosomal cyclic peptides.

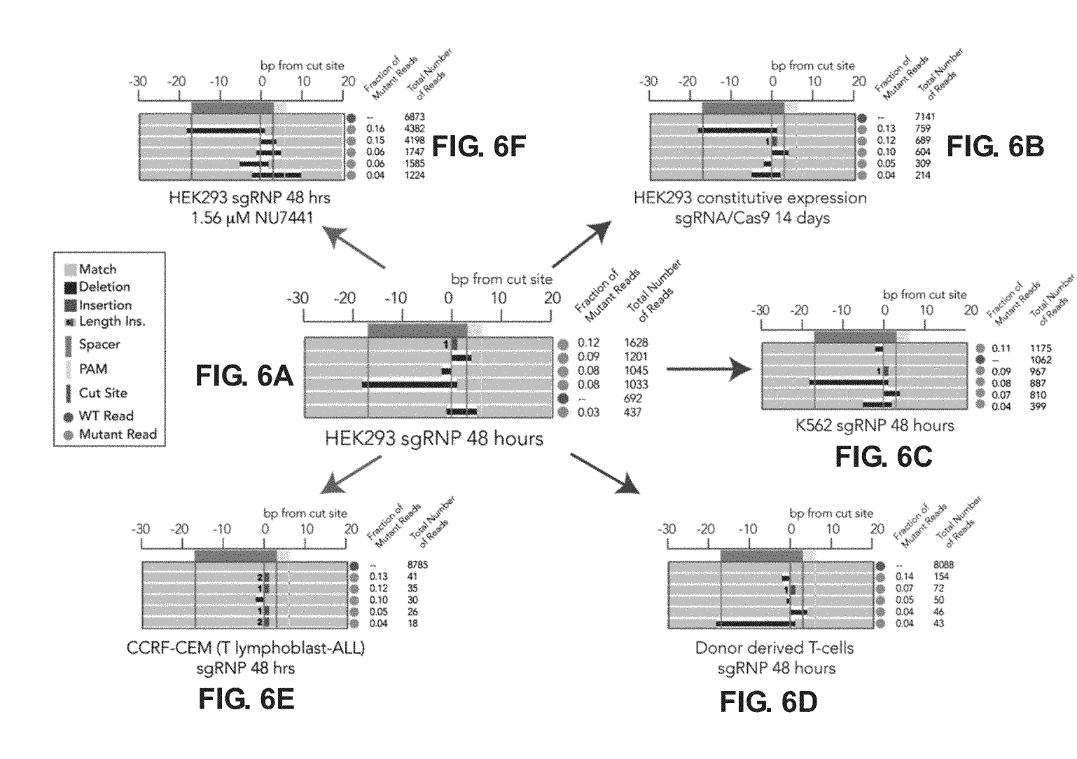

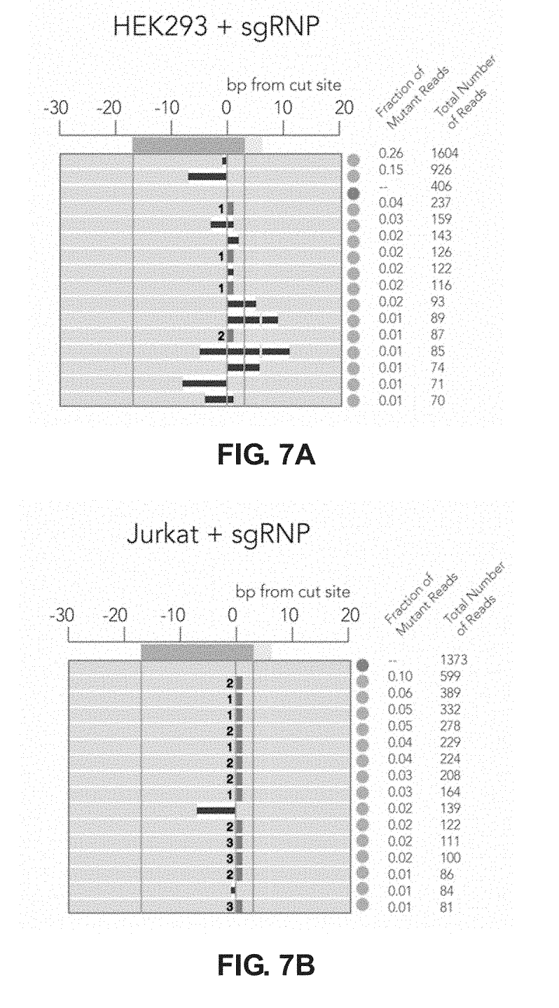

The techniques described herein for library generation can be designed to manipulate DNA repair pathways, such as by favoring particular repair mechanisms, or for example, favoring insertion-biased DNA. In this regard, it has now been demonstrated that the distributions of DNA repair outcomes at Cas9-mediated double-strand breaks (DSBs) are, in fact, nonrandom and dependent on the target site sequence (see, Example 1A and FIGS. 6A-6F and 7A-7B). Large scale computational analyses of DSBs indicated the same DNA repair patterns were observed across experimental replicates, cell lines and reagent delivery methods.

Additionally, compounds can be used to alter DNA repair pathways. For example, it has been shown that compounds that suppress c-NHEJ alter DNA repair landscapes, and can favor MMEJ repair outcomes. See, van Overbeek et al., Molecular Cell (2016), http://dx.doi.org/10.1016/j.molcel.2016.06.037. NHEJ is initiated when free DNA ends are bound by Ku70 and Ku80, which recruit the catalytic subunit of DNA-dependent protein kinase (DNA-PKcs). The resulting complex, known as the DNA-dependent protein kinase (DNA-PK) complex, phosphorylates downstream targets leading to activation of the DNA damage response and initiation of NHEJ. Thus, suppression of the NHEJ key enzymes Ku70, Ku80, or DNA Ligase IV inhibit DNA-PK and can be used in the present methods to modulate DNA repair outcomes by inhibiting NHEJ. Such inhibitors include without limitation, NU7441 (Leahy et al., Bioorg. Med. Chem. Lett. (2004) 14: 6083-6087); KU-0060648 (Robert et al., Genome Med (2015) 7:93); DNA Ligase IV inhibitor, Scr7 (Maruyama et al., Nat. Biotechnol. (2015) 33:538-542); NU7026 (Willmore et al., Blood (2004) 103); anti-EGFR-antibody C225 (Cetuximab) (Dittmann et al., Radiother and Oncol (2005) 76:157), and the like.

Moreover, certain cell lines can also contribute to insertional bias in DNA repair. Such cell lines include, without limitation, human lymphoblastic cell lines such as Jurkat and CCRF-CEM cells. Jurkat and CCRF-CEM are human lymphoblast cell lines and display altered DNA repair patterns compared to other cell lines, including primary T cells, to which they are most similar. Both of these cell lines demonstrate a bias towards small insertions (see, FIGS. 6A-6F and 7A-7B).

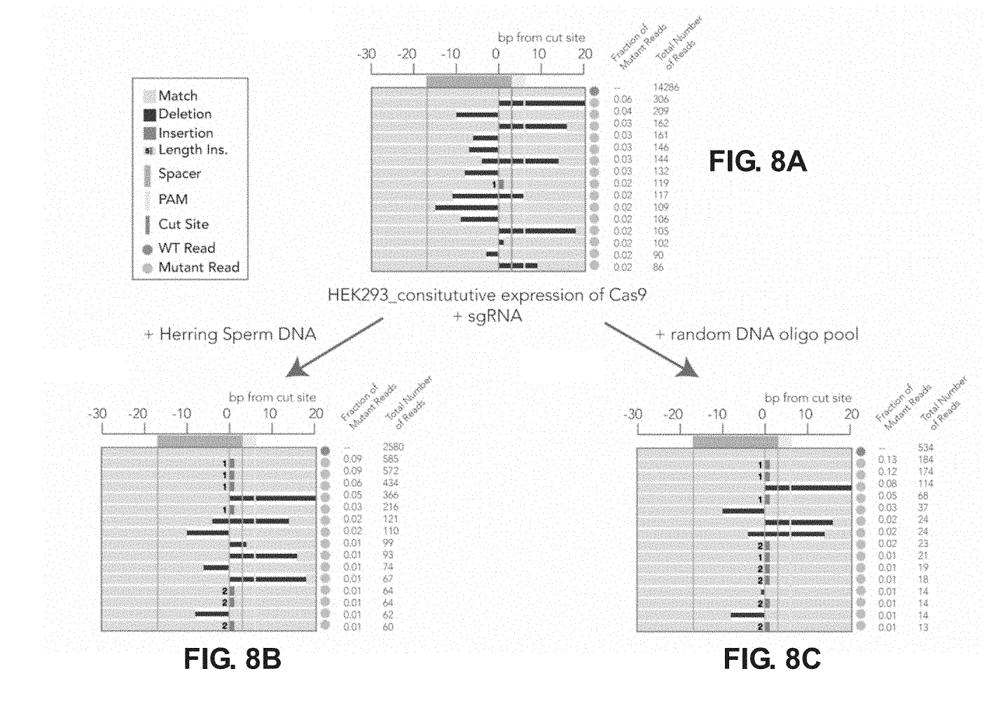

Additionally, introduction of exogenous DNA during the transfection of CRISPR reagents results in an insertional bias in the resulting DNA repair. See e.g., Example 1D wherein transfection of cells, such as human cells, with for example, random short oligomers ranging in length from about 3-50 base pairs, e.g., 3, 4, 5, 6, 7, 8, 9, 10, 11, 12, 13, 14, 15, 16, 17, 18, 19, 20 base pairs, such as 5-30, e.g., 5-25, 5-20, or any integer within these ranges, or other exogenous DNA, such as herring sperm DNA, shifts DNA repair patterns toward small insertions (FIGS. 8A-8C). Thus, larger exogenous DNA, such as up to about 5000 base pairs in length, e.g., up to 3000, 2500, 2000, 1500, 1000, 500, 50, or any integer within these ranges, will find use herein. Thus, introduction of exogenous DNA during the transfection of CRISPR reagents results in an insertional bias in the resulting DNA repair and this bias can be used to manipulate repair outcomes and generate diverse protein engineering libraries. Without being bound by a particular theory, the introduction of exogenous DNA may mimic DNA breaks and lead to a hyperactive DNA damage response that disrupts the process of DNA repair, as previously observed at the molecular level (Quanz et al., PLoS ONE (2009) 4:e6298 and Croset et al., Nucl. Acids Res. (2013) 41:7344-7355). As shown herein, DNA repair disruption at the genomic level resulted in a bias toward small insertions.

Disruption of DNA repair in a manner advantageous for the generation of diverse protein engineering libraries may also be achieved by other means, including but not limited to, exogenous expression of proteins or protein segments that activate the DNA damage response. See, Toledo et al., Genes & Development 22.3 (2008): 297-302. Such proteins or protein segments include the ATR stimulating fragment of TopBP1 (amino acids 978-1286 of the human protein) or full-length TopBP1 or other protein activators of the DNA damage-response kinases ATM, ATR or DNA-PK.

Insertional bias in DNA repair results in many more repair classes than the typical DNA repair pattern at a given site. Each of these repair classes represents a unique gene sequence that can result in a protein with a new or altered function. Thus, introducing this type of diversity at multiple sites within a gene allows for the rapid generation of dynamic DNA libraries in cells. To do so, multiple guide RNA/Cas complexes, such as 2, 3, 4, 5, 6, 7, 8, 9, or 10 or more guide RNA/Cas complexes can be used to transfect cells to yield an extremely large number of possible unique, in-frame gene sequences (see, Example 1E).

In certain embodiments, insertion-biased DNA repair, as described above, is used to create molecular libraries for discovery of proteins with new or altered functions. Human cell lines, such as lymphoblast cell lines that display insertional bias, such as but not limited to Jurkat and CCRF-CEM cell lines, can be used to create molecular libraries of endogenously expressed genes, e.g., by delivering a guide polynucleotide that targets a selected protein coding region in genomic DNA present in the cell. Double-strand breaks can then be produced using a programmable endonuclease. For example, guide polynucleotides, such as sgRNAs, designed to target particular regions, can be delivered to the cell. If the cell constitutively expresses a Cas endonuclease, such as Cas9, Cpf1, or the like, the Cas endonuclease will then be recruited to the target site to cleave the DNA. If the cell does not express a Cas endonuclease, complexes of Cas proteins, such as Cas9 proteins, and guide RNAs, such as sgRNAs (sgRNA/Cas9 complexes) are delivered to the cells to make double-strand breaks, thereby triggering the DNA repair pathways in the cells to create diverse molecular libraries. The libraries are then screened using methods well known in the art, such as using high-throughput screening techniques including, but not limited to, flow cytometry techniques, including, without limitation, fluorescence-activated cell sorting (FACS)-based screening platforms, microfluidics-based screening platforms, emulsion/droplet-based analysis methods, and the like. These techniques are well known in the art and reviewed in e.g., Wojcik et al., Int. J. Molec. Sci. (2015) 16:24918-24945.

Insertion-based DNA repair can be directed precisely toward sites of interest within a selected protein coding region. The guide polynucleotides, such as sgRNAs, can be designed to target any DNA sequence containing the appropriate PAM necessary for each Cas endonuclease, such as Cas9, Cpf1 and the like. Additional PAMs can also be created in the target DNA using a type of codon optimization, where silent mutations are introduced into amino acid codons in order to create new PAM sequences. For example, for strategies using Cas9, which recognizes an NGG PAM, a CGA serine codon can be changed to CGG, preserving the amino acid coding but adding a site where double-strand breaks can be introduced. Moreover, computational analysis on small insertions shows that C's and G's are inserted with high frequency. This can be used to create new PAMs and thus new sites for diversity generation. The entire coding region or parts of the coding region can thus be optimized with suitable PAM sites on the coding and non-coding strand for insertion-based DNA repair after nuclease cleavage. PAM optimized DNA sequences can then be manufactured, e.g., commercially, and cloned into suitable vectors

In an exemplary embodiment, the methods described herein are useful for creating molecular libraries of an endogenous gene, such as for generating T cell receptor (TCR) libraries, in order to engineer cytotoxic T lymphocytes (CTLs) specific for novel oncogenic or infectious antigens. TCRs are heterodimers composed of two different protein chains. Most TCRs include an alpha (α) chain and a beta (β) chain, encoded by TRA and TRB, respectively. A smaller percentage of TCRs include gamma (γ) and delta (δ) chains, encoded by TRG and TRD, respectively. α/β TCRs recognize antigens bound to WIC molecules whereas TCR γ/δ can directly recognize antigens in the form of intact proteins or non-peptide compounds. See, e.g., Allison et al., Nature (2001) 411:820-824 for a description of the structure of γ/δ TCRs.

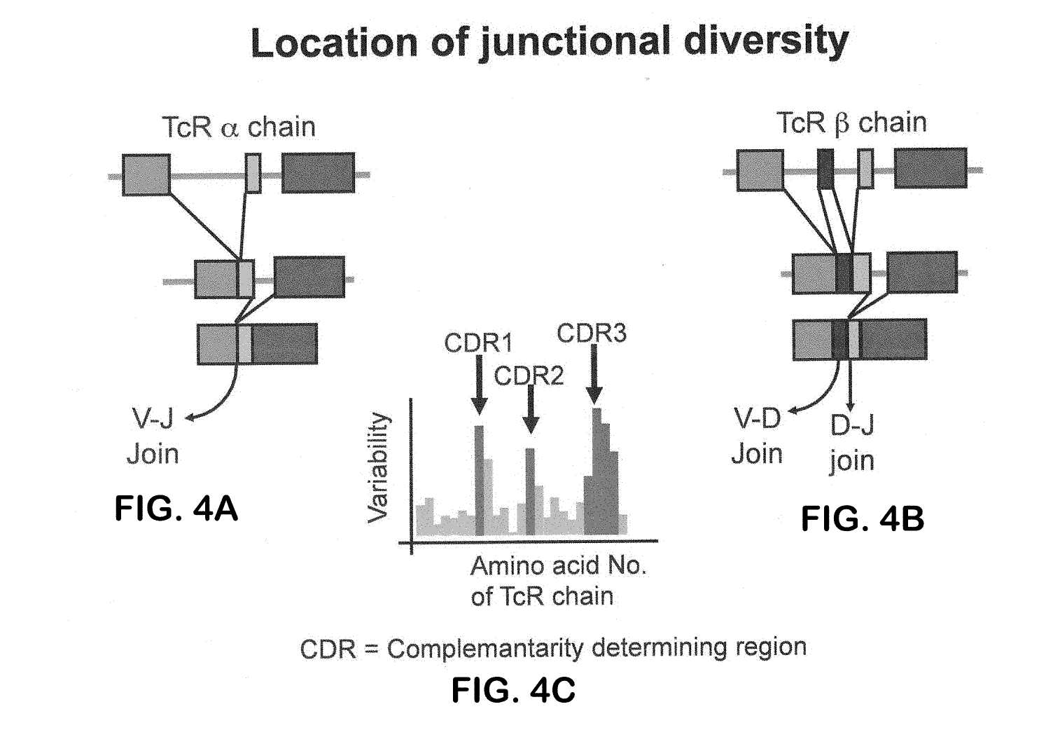

In a representative embodiment, the three complementary determining regions (CDRs) in each of the TCR chains, such as the TCRα and TCRβ chains, are targeted. These regions are known to interact with the antigen:WIC complex (major histocompatibility complex) and contribute to TCR specificity and affinity (see, FIG. 4A, FIG. 4B, and FIG. 4C). The amino acid locations of the human CDR regions for the a subunit are as follows: CDR1: 24-31; CDR2: 48-55; CDR3: 93-104. The amino acid locations of the human CDR regions for the β subunit are as follows: CDR1: 26-31; CDR2 48-55; CDR3: 95-107.

Guide RNAs that target these regions can be readily designed based on the known sequences of human CDR1, CDR2 and CDR3. Hundreds of such sequences are reported in the International Immunogenetics Information System database (IMGF/LIGM-DB); Giudicelli et al., Nuc. Acds Res. (2006) 34:D781-D784. TCR libraries can be created in human cell lines, such as lymphoblastic cell lines and screened via flow cytometry using a peptide-MHC binding assay. Selected clones are expanded and subjected to iterative rounds of editing and positive and negative selection. The TCR::antigen binding affinity can also be determined using Surface Plasmon Resonance for selection of desired TCR sequences. These sequences can be cloned into primary human T cells to demonstrate cytotoxic capabilities.

In this way, error-prone DNA repair can be used to reprogram the specificity of endogenous TCRs in vivo. CRISPR-Cas-mediated double strand breaks are targeted to specific regions of the TCR, such as the TCRα, TCRβ, TCRγ and/or TCRδ chains, as described above. The error-prone DNA repair that ensues generates a cellular library of diverse TCRs, which can then be screened to identify those cells that maintain TCR expression but have altered binding specificity.

The libraries can be created in human cell lines, such as lymphoblast cell lines that display insertional bias, such as but not limited to the Jurkat T cell lymphoma cell line, which endogenously expresses the TCR and associated signaling proteins. Jurkat cells exhibit a DNA repair pattern biased towards insertions. This insertion-biased repair leads to the generation of a library with more functional diversity than a library that has a typical distribution of insertions and deletions.

As explained above, the screening method can utilize flow cytometry. In this technique, an antibody that recognizes the TCR constant region, such as, but not limited to, a TCR antibody clone, such as a TCR α/β antibody clone, e.g., IP26 or WT31; (both available from Thermo Fisher Scientific (Waltham, Mass.)), is used to screen the library for cells that maintain TCR surface expression. A fluorescently-tagged peptide-MHC complex wherein the peptide represents the antigen of interest, can be used to screen the library for cells that have the desired antigen specificity in a commonly used assay, such as a tetramer assay. In one particular embodiment described in the examples, the variable region of the β-chain of the TCR in Jurkat cells, which is specified by the TRVB12-3 gene, is targeted. Multiple sgRNPs can be delivered individually to Jurkat cells and the resultant libraries screened with a TCR α/β antibody, e.g., the IP26 antibody as well as an antibody specific to the targeted region. TCR sequences with preserved IP26 antibody staining but abolished binding of the antibody to the variable region can be produced.

Clonal cell lines generated from these polyclonal pools and the mutant TCRs can be sequenced and the functionality of these reprogrammed TCRs can be assessed, such as by using a cytokine release assay. Properties such as binding affinity and signal strength can be determined and tuned using base-editing techniques, such as site-directed mutagenesis techniques or by using dCas9-AID fusion proteins (Komor et al, Nature (2016) 533:420-424). When targeted to the entire coding region of the TCR, base editing can be used to restore or refine the signaling properties of the engineered TCRs. Once a TCR with desired properties is identified, it can be cloned and expressed in primary cells.

The reprogrammed TCRs can be used in TCR immuno-oncology therapies, to access tumor antigens, such as cell surface and intracellular antigens. Additionally, the technology can be expanded to non-TCRs and used to engineer other antibodies, and proteins expressed in mammalian cells. For example, these techniques can be used to engineer new antibodies to tumor antigens and neoantigens and these new antibodies can then be incorporated into a CAR (chimeric antigen receptor) for adoptive therapies. CARs are hybrid receptors that are fusions of single-chain variable fragments (ScFv) from antibodies specific to tumor-associated antigens and T cell signaling domains. When ectopically (virally or otherwise) expressed in primary T cells, CARs allow for T cell activation in the presence of tumor antigen binding. This type of therapy provides new approaches to the treatment of blood cancers and solid tumors. Methods for engineering CAR T cells are known and described in, e.g., Lin et al., Cell (2017) 168:724-740.

The techniques described herein enable new CAR designs. For Example, CRISPR-Cas-mediated diversification of immunoglobulin genes in human lymphoblastic cell lines, such as Jurkat cells, paired with screening against tumor associated antigens or patient-specific neoantigens, can result in the discovery of new antibodies. These antibodies can then in used in CARs as antigen binding domains. These methods apply not only to traditional CAR designs but to newer dual specificity and other designs as well.

The above methods of TCR engineering provide several advantages over other TCR engineering techniques. For example, current technology for TCR engineering is to retrieve TCRs of interest from the natural repertoires in cancer patients or immunized mice (Barrett et al., J. Immunol. (2015) 195:755-761). This approach is limited by several factors, including the differences between the murine and human immune responses, as well as the fact that TCRs with high affinity for self-antigens are typically deleted to prevent autoimmune disease. The above described technique, on the other hand, enables engineering of TCRs that are not encoded in the germline repertoires such as very long or very short variable regions and D-elements.

Other approaches for TCR engineering take advantage of yeast display technology. However, such approaches are limited because TCR α and β chains must be expressed individually on the yeast cell surface and thus the libraries screened separately. The technology described herein allows for native expression of the dimeric TCR during the mutagenesis and screening processes. In addition, as Jurkat cells are T cells and express all of the necessary downstream signaling molecules, phenotypic responses including signaling can be used in screening in addition to binding.

In another exemplary method, insertion-biased repair can be used to engineer Vβ8, a T cell receptor subunit that mediates binding to staphylococcal enterotoxin B (SEB). Native Vβ8-SEB binds with low affinity. Vβ8 proteins with higher binding affinity can be engineered using these techniques. The edited cells, produced as described above, are screened by an SEB binding assay using flow cytometry as described in Sharma et al, Protein Engineering, Design & Selection (2013) 26: 781-789, where binding of biotinylated SEB is detected using fluorophore-conjugated streptavidin reagents. In order to select for Vβ8 proteins with increased binding affinity for SEB, the stringency of the binding assay is increased in each round by decreasing the concentration of SEB used.

In one embodiment of this procedure, populations of cells that show some binding activity are sorted after each round of editing, expanded and re-transfected for iterative rounds of editing. In another embodiment of this procedure, cells are re-transfected and edited multiple times before screening at the population level. When a population reaches a desired level of binding activity, individual clones are sorted, expanded and sequenced to recover the mutations that result in enhanced function.

Similarly, human cell lines, such as lymphoblast cell lines that display insertional bias, such as but not limited to Jurkat and CCRF-CEM cell lines, can be used to create molecular libraries of exogenously expressed genes. For example, a donor construct, such as a multicistronic vector (also termed an “insertion cassette” herein), encoding the exogenous protein of interest and a selectable marker, such as for antibiotic resistance, e.g., blasticidin, puromycin, neomycin antibiotic resistance, and the like, is inserted into an integration locus, such as Adeno-Associated Virus Integration Site 1 (AAVS1). The AAVS1 locus allows stable, long-term transgene expression in many cell types. Methods for integrating exogenous genes into AAVS1 are well known in the art and described in, e.g., Yanez et al., Methods (2016) 101:43-55. The methods employ programmable endonucleases, such as CRISPR-Cas nuclease technologies, TALENs, ZFNs, meganucleases, MEGA-TALs, Ago, etc. to produce double-strand breaks at the appropriate AAVS1 insertion sites. Other loci can also be used, such as, but not limited to, the CCR5 locus and the human orthologue of the mouse Rosa26 locus. Exogenous safe harbor sites can also be added, such as addition of a human chromosome, to allow exogenous gene insertion without disruption of the endogenous cellular genome.

For example, as with the methods described above, guide polynucleotides, such as sgRNAs, designed to target the PAM sequences adjacent to the AAVS1 insertion site, can be delivered to the cell. If the cell constitutively expresses a Cas endonuclease, such as Cas9, Cpf1, or the like, the Cas endonuclease will then be recruited to the target site to cleave the DNA. If the cell does not express a Cas endonuclease, complexes of Cas proteins, such as Cas9 proteins, and guide RNAs, such as sgRNAs (sgRNA/Cas9 complexes) are delivered to the cells to make double-strand breaks. After selection and verification of the insert, single cell clones are expanded to create a stable cell line expressing the gene of interest at the desired locus. Guide RNAs, such as sgRNAs, are designed to target the insert and are complexed with a Cas protein, such as Cas9, and the complexes are introduced into the cells, such as by electroporation. The cells are grown in the presence of the appropriate antibiotic in order to select for sequences with in-frame insertions of the gene of interest. Cells are then sorted using e.g., FACS, to isolate cells with the desired properties. The cells can then be expanded and re-transfected with additional guide complexes to introduce further diversity and this process can be repeated iteratively until a population with the desired properties is obtained. Single cell clones are sorted from the population, expanded and sequenced to recover the mutations that resulted in the desired function.

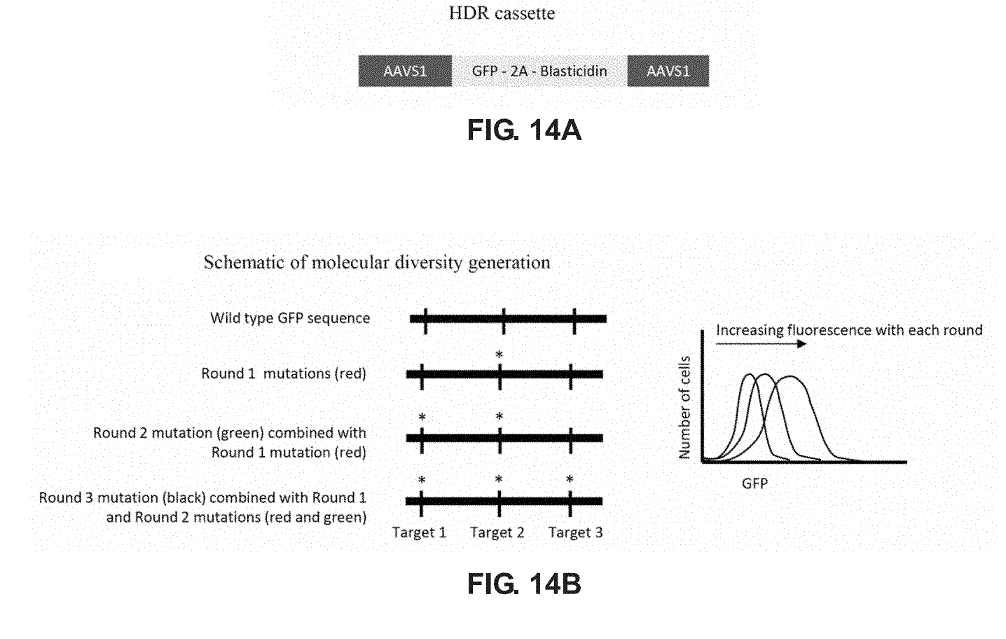

In one exemplary embodiment, the above technique is used to engineer an Aequorea victoria Green Fluorescent Protein (GFP) with enhanced fluorescent properties. A schematic of this method is shown in FIGS. 14A and 14B. In this embodiment, a homology-directed repair cassette encoding wild-type A. victoria GFP fused to a 2A self-cleaving peptide and a blasticidin-resistance sequence and flanked by homology arms (depicted in FIG. 14A) is inserted into the AAVS1 locus in a lymphoblastic cell line, as described above. After selection and verification of the insert, single cell clones are expanded to create a stable cell line expressing the GFP at the desired locus. Guide RNA/Cas complexes targeting the GFP insert, are introduced into cells and cells are grown with blasticidin for selection of sequences with in-frame insertions in GFP. Cells are then sorted using FACS to isolate those with enhanced green fluorescence. The cells are expanded, etc., as described above until a population with the desired fluorescent properties is obtained. The DNA sequences of the enhanced GFP variants are then obtained by sequencing the GFP loci in the sorted cell population.

In addition to the insertional bias methods described above, which use human lymphoblastic cell lines, insertional bias can also be created in any cell line or primary cell type by delivery of exogenous DNA to create molecular libraries of endogenously expressed genes. As explained above, delivery of exogenous DNA, such as small, single-strand oligomers or Herring Sperm DNA, results in insertional bias. Thus, in this method, targeted double-strand breaks are introduced by using programmable nuclease systems as described above. For example, Cas-mediated breaks, such as Cas9-mediated double-strand breaks, can be introduced by delivery of sgRNAs into cells that constitutively express Cas9, or by delivery of sgRNA/Cas9 complexes. Exogenous DNA, such as random oligos of the lengths described above, are concurrently transfected into cells, resulting in an insertional bias in DNA repair at the cut site directed by the sgRNA. See, FIG. 8.

Thus, the sgRNAs can be targeted to endogenously expressed genes in any cell line, including but not limited to immunoglobulin genes, T cell receptors, cytokine receptors, cell adhesion molecules, nucleic acid binding proteins, G-protein coupled receptors or enzymes. An appropriate screen is used, such as a binding assay, and cells expressing mutated genes coding for proteins with the desired function can be separated, cloned and the sequence isolated.

Similarly, exogenous DNA, such as small oligos, etc., described above, can be used to create libraries of a selected exogenous gene in any cell line or primary cell type using the insertional bias created when such exogenous DNA is transfected along with guide RNA/Cas complexes. In this embodiment, a donor construct, for example a multicistronic vector, encoding the exogenous protein of interest and a selectable marker, such as for antibiotic resistance, is inserted into an integration locus, such as AAVS1. After selection and verification of the insert, single cell clones are expanded to create a stable cell line expressing the cassette at the desired locus. Guide RNAs are designed to target the entire coding region of the protein of interest, or specific regions predicted to be involved in protein function. Guide RNA/Cas complexes, along with exogenous DNA, such as small oligos, are concurrently transfected into the stable cell line and cells expressing mutated genes coding for the selected proteins are screened, such as using a recombination or DNA cleavage assay, separated, cloned and the sequence determined.

In particular embodiments, the protein can be a recombinase, such as from the Cre family, a transposase, a programmable nuclease, such as a Cas nuclease, or almost any selected protein, either wild-type or engineered protein. A cassette, such as detailed above, flanked by homology arms and encoding the protein of interest fused to a 2A self-cleaving peptide or IRES cite, and a blasticidin resistance sequence, is inserted into the AAVS1 integration locus and stable cell lines are created that harbor the cassette at the desired locus. Alternatively, an engineered protein expression library can be generated by various methods including those outlined in Example 4B for subsequent expression in a cell system such as mammalian, plant or bacterial cells. In this embodiment, the expression library is introduced such that a heterogeneous population of cells is derived, each expressing a different protein variant. In both embodiments, guide RNAs are designed to target the entire coding region of the protein of interest, or specific regions predicted to be involved in protein function. Guide RNA/Cas complexes, along with exogenous DNA, such as small oligos, are concurrently transfected into the stable cell line and cells expressing mutated genes coding for the selected proteins are screened, such as using a recombination or DNA cleavage assay, separated, cloned and the mutant sequence determined. If Cre is the recombinase chosen for engineering, for example, molecular libraries of the protein are screened for mutants that can recombine novel DNA sequences. These novel recombination sequences can be unique sequences within safe harbor sites such as AAVS1.

The screen entails transfecting the cells with a plasmid encoding a fluorescent marker such as Green Fluorescent Protein (GFP) and a stop codon flanked by recombinase recognition sites, similar to the Substrate Linked Protein Evolution system used in Buchholz et al., Nature Biotechnology (2001) 19:1047-1052. A mammalian promoter is immediately upstream of the 5′ recombinase recognition site and a different fluorescent protein such as mCherry, is immediately downstream of the 3′ recombinase recognition site. All cells that express the plasmid will emit green fluorescence. If a mutant recombinase protein can recognize the given DNA sequences, the GFP gene is excised and the cells are no longer green. Such recombination will also result in expression of the second, red fluorescent protein, thereby creating a system in which the ratio of green and red fluorescent cells allows for the calculation of recombination efficiency.

In another embodiment, diverse libraries for protein engineering can be prepared using a site-directed base substitution system. These methods for evolving/engineering a protein of interest maintain the proper reading frame using DNA base substitution in the open reading frame of the protein of interest and can be used to generate diversity within any given stretch of DNA sequence in an orthogonal manner to insertion-biased repair diversity, or protein shuffling as described herein.

For example, base substitution activity by a cytidine deaminase is regulated in mature B-cells and can occur in a dysregulated manner in a phenomena termed kataegis (D'Antonio et al., Cell Reports (2016) 16:672-683; Casellas et al., Nat Rev Immunol (2016) 16:164-176). The B cell specific activation-induced cytidine deaminase (AID) has been used in vitro to mature and expand the diversity of antibodies in mammalian display systems through base substitution (Bowers et al., Proc. Natl. Acad. Sci. USA (2011) 108:20455-20460; Bowers et al., Journal of Biological Chemistry (2014) 289:33557-33567).

In human cells, base editing via cytidine deaminases does not require the introduction of DNA double strand breaks and as such is correlated with very low frequency indel formation. Cytidine deaminases first convert cytosine residues to uracil residues which can then be converted to residues other than cytosine by means of mismatch repair mechanisms thus leading to mutagenesis.

In the first step toward mutagenesis, the converted cytosine can be recognized by uracil DNA glycosylase enzymes which facilitate restoration of the parental cytosine residue. In particular, uracil DNA glycosylases, also known as “UDG” or “UNG,” prevent mutagenesis by eliminating uracil from DNA molecules by cleaving the N-glycosylic bond and initiating the base-excision repair (BER) pathway. For a review of BER, see, e.g., Seeburg et al., Trends in Biochem. Sci. (1995) 20:391-397; Kim et al., Curr. Mol. Pharmacol. (2012) 5:3-13. Uracil bases occur from cytosine deamination or misincorporation of dUMP residues. After a mutation occurs, the inclusion of uracil propagates through subsequent replication steps. Mismatched guanine and uracil pairs are separated, and DNA polymerase inserts complementary bases to form guanine-cytosine (GC) pairs in one daughter strand and adenine-uracil (AU) pairs in the other strand. UNG excises uracil in both AU and GU pairs to prevent propagation of the base mismatches.

Thus, in order to enhance mutagenesis, cell lines can be established with suppressed gene expression of UNG via genetic knockout, or other genetic manipulation, thereby promoting cytidine deaminase-mediated base editing. By “suppressed expression” is meant lowered expression relative to a cell with normal UNG expression. Thus, suppressed expression does not mean that none of the gene product is expressed. Rather, UNG expression can be suppressed if it is reduced by e.g., 50%, 75%, 80%, 85%, 90%, 95%, 99%, 100%, or any percentage within these ranges, as compared to a cell with normal UNG expression levels. Thus, in some cases, e.g., when the gene is completely knocked out, expression levels of UNG can be zero.

Such cell lines can be produced using technologies well known in the art, such as CRISPR technology as described herein. See, also, Liang et al., J. Biotech. (2015) 208:44-53. Additionally, CRISPR-based human UNG knockout kits are commercially available from e.g., OriGene Technologies, Inc. (Rockville, Md.). Cell lines with UNG knockouts also exist and include, without limitation, the HAP1 cell line (Horizon Discovery, Cambridge, UK), wherein the UNG gene has been edited by CRISPR/Cas to contain a 28 bp deletion in a coding exon of UNG. Other methods of producing targeted genetic knockouts are well known and include, without limitation, the use of non-CRISPR targeted nucleases, such as ZFNs (see, e.g., Santiago et al., Proc. Natl. Acad. Sci. USA (2008) 105:5809-5814), Transcription activator-like effector nucleases (TALENs), meganucleases, MEGA-TALs, Argonaute (Ago), and others known to one of skill in the art.

As explained above, in the second step towards mutagenesis, mismatch repair (MMR) mechanisms promote the conversion of the uracil-guanine base pairing to traditional DNA base pairing which may differ from the parental base sequence. Thus, MMR and hence mutagenesis, can be promoted by over expressing protein components of the MMR machinery. Such components include, but are not limited to PMS2, MLH1, MLH3, MSH2, MSH3, MSH6. For a review of MMR see, Jiricny, J. Nature Reviews Molec. Cell Biol. (2006) 7:335-346. Over expression of MMR components have been observed in human cancers and are correlated with enhanced mutagenesis.

Accordingly, cell lines can be produced and used that overexpress genes coding for components of the MMR pathways. This can be achieved by preparing vectors including the particular MMR component of interest, such as a gene encoding PMS2, and transforming, transfecting or transducing cells with these vectors. The PMS2 gene present in the vector can be previously mutated such that it possesses enhanced activity. Alternatively, the gene can be associated with a heterologous promoter providing enhanced expression activity as compared to the naturally occurring promoter. Such promoters are well known in the art and several promoters are described in detail below. Transcriptional enhancer elements can also be present to increase the function of the promoter, thus increasing transcription of the MMR gene of interest. A translational enhancer can also be present. The recombinant vector is then used to transform, transfect or transduce an appropriate cell, using techniques well known in the art, such as those described in detail below. The MMR protein can be expressed transiently such that over expression occurs for a short time if desired, or cell lines can be designed to provide for stable gene expression. Techniques to achieve expression in vitro are well known in the art.

Suitable cells for use in the present methods include prokaryotic and eukaryotic cells, such as bacterial, yeast, insect and mammalian cells. For example, human lymphoblast cell lines such as Jurkat and CCRF-CEM cells, will find use with the present methods. In some embodiments, the cell lines include both constructs for repressing expression of uracil DNA glycosylases, as described above, and constructs for over expressing components of the MMR pathway. In other embodiments, the cells include either constructs for repressing expression of uracil DNA glycosylases or constructs for over expressing components of the MMR pathway.

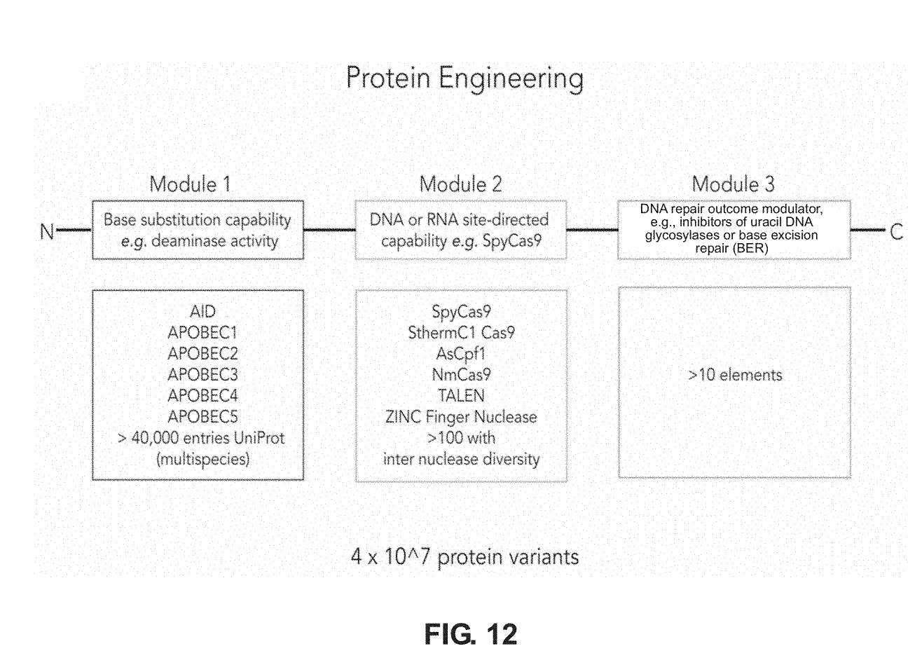

These cell lines can then be used in combination with a construct that includes multiple protein-encoding sequences useful for site-directed base substitution. One such construct is depicted in FIG. 12. As shown in FIG. 12, 4×10⁷ protein variations can be engineered containing, for example, three modules, where Module 1 encodes for a molecule with base substitution capabilities such as deaminase activity (Knisbacher et al., Trends in Genetics (2016) 32:16-28). Such molecules include, without limitation, activation-induced cytidine deaminase (AID), any of the various Apolipoprotein B mRNA editing enzymes (APOBE), such as, but not limited to APOBEC1, APOBEC2, APOBEC3, APOBEC4, APOBEC5, and the like. The APOBE enzymes may be derived from any species that includes the appropriate APOBE homologs. For example, rat APOBEC1 has been seen to have high activity in human cells. See, e.g., the UniProt database for a listing of multiple such enzymes.

Module 2 provides site-directed DNA binding capabilities such as encoding programmable endonucleases, including but not limited to, Cas endonucleases, such as SpyCas9, SthermC1 Cas9, AsCpf1, NmCas9, TALENs, ZFNs, etc. Additionally, molecules can be used that retain site-directed binding capability but may or may not retain nuclease activity. For example, Cas9 mutants, with one or both of the nuclease domains mutated such that DNA cleavage activities are hampered, but site-directed binding capabilities remain, can also be used. Such molecules are known as “dCas9,” “catalytically inactive,” “catalytically dead,” or “dead” Cas9.” This is typically accomplished by mutating both of the two catalytic residues (D10 in the RuvC-1 domain, and H840 in the HNH domain, numbered relative to S. pyogenes Cas9) of the gene encoding Cas9. For example the mutation can be a substitution of A for D in the RuvC-1 domain (i.e., D10A). Similarly, the mutation can be a substitution of A for H in the HNH domain (i.e., H840A).

Module 3 encodes modulators of DNA repair activities such as inhibitors of uracil DNA glycosylase or BER (base-excision repair). Inhibitors of uracil DNA glycosylases can function by altering the regulation of transcription of uracil DNA glycosylases. These inhibitors may be endogenous regulators of transcription factors such as AP-1 (a regulator of UNG gene expression) or CRISPR interference (CRISPRi), a genetic perturbation technique that allows for sequence-specific repression or activation of gene expression, or RNAi mediated inhibition. By “inhibitor” is also meant a loss of gene expression due to site-directed endonuclease activity at the gene locus yielding a functional knock-out. Inhibitors of uracil DNA glycosylases may also be chemical inhibitors such as small molecules that bind to or otherwise interfere with the ability of uracil DNA glycosylases to perform their function. Inhibitory proteins may also function to inhibit uracil DNA glycosylases analogous to the inhibition of uracil DNA glycosylases in bacteria by inhibitory proteins expressed by bacteriophage. Such proteins can be over-expressed in cells by preparing vectors including the inhibitory component of interest and transforming, transfecting or transducing cells with these vectors.

Inhibitors of BER can alter the function of any component of the BER pathway, including but not limited to; UNG, TDG, SMUG1, MBD4, AAG, MPG, APE1, POL beta, LIGIII alpha, XRCC1, OGG1, NTH1, NEIL1/2, PNKP, PCNA, Pol delta, Pol epsilon, FEN1, LIGI. Inhibition may be via mis-regulation of transcription or functional knock-out as described above, or by chemical inhibition. For example, chemical inhibition of OGG1 activity has been shown using small molecules that inhibit Schiff base formation during OGG1-mediated catalysis. See, e.g., Donley et al. ACS Chem. Biol. (2015) 10:2334-2343). Inhibition of BER may also be achieved by inhibitory protein over-expression whereby protein components known to inhibit the function of any component of the BER pathway may be over-expressed in cells by preparing vectors including the inhibitory component of interest and transforming, transfecting or transducing cells with these vectors.

The modules present in the recombinant construct can be regulated by separate promoters, or can be present in a multicistronic configuration under the regulation of a single promoter. In this case, each coding sequence will typically include its own Shine-Dalgarno sequence and start codon. To achieve expression of multiple genes using a single promoter, internal ribosome entry sites (IRES elements) can be included that permit the translation of two or more open reading frames from a single mRNA. Many such IRES elements are known. See, e.g., Hellen et al. Genes Dev. (2001) 15:1593-612. Alternatively, 2A self-cleaving peptides can be used in the multicistronic vectors. These peptides are short (about 20 amino acids in length) and produce equimolar levels of multiple genes from the same mRNA. 2A peptides include T2A, P2A, E2A, and F2A. See, e.g., Scymzczak et al., Cold Spring Harb. Protoc. (2012) 2012:199-204 for a description of methods for the design and construction of such multicistronic vectors.

Using the above techniques, a subset of the engineered proteins can be screened for site-directed base substitution activity and candidates used to further evolve and diversify a protein of interest as described in Example 4.

In another embodiment, diverse libraries can be produced using recombinase-mediated protein diversification methods. In this embodiment, a cell line of interest can be engineered to produce a “protein diversification cell line” that harbors an artificial recombination locus. This locus supports the integration of protein modules (gene fragments). The number of protein modules that can be inserted depends on the recombination locus, the size of the fragment inserts, etc. but will typically be from 2-20. A broad range of gene fragments can be accommodated by combining known site-specific recombinases, transposases or integrase enzymes such as, but not limited to, Flp, Cre, psi C31, MuA, Tn5, Tn10, Sleeping Beauty and PiggyBac transposases or variants of known enzymes engineered to recognize new recognition sequences. The protein diversification cell line is transfected with protein modules present in gene fragment donor libraries discussed below, along with a recombinase expression vector that includes recombinases that drive recombination at the recombination acceptor sites present in the integrated artificial recombination cassette, to yield diverse protein products for use in downstream assays.

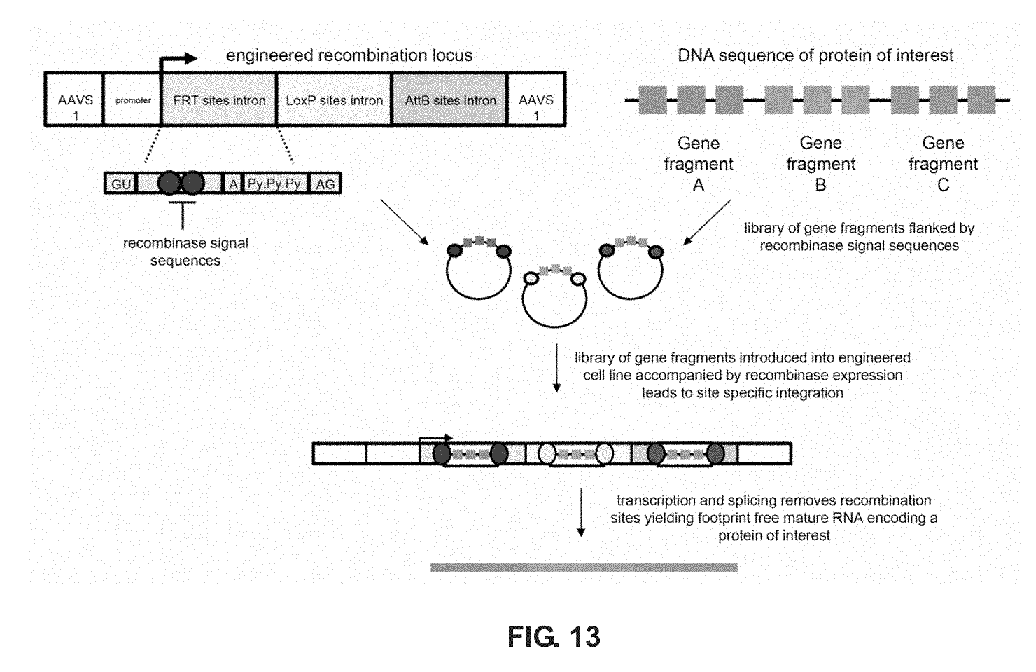

One representative method is detailed in Example 4 and shown in FIG. 13. In this representative method, the recombination locus is a large, double-stranded DNA fragment with 5′ and 3′ flanking homology arms for insertion into the AAVS1 locus of a cell line of interest, such as HEK293 cells. As shown in FIG. 13, the recombination locus includes a 5′ AAVS1 homology region, followed by a promoter sequence to drive transcription through the three downstream recombination regions which are, in 5′ to 3′ order, paired FRT sites, paired LoxP sites and paired AttB sites. However, these sites can occur in any order. Moreover, the recombination loci within the engineered cassette are not limited to FRT, loxP and attB sites, but can include variants thereof as well as other recombination loci including altered recombinase sites recognized by engineered recombinases such as Cre (Buchholz et al., (2001) Nature Biotech 19:1047-10529; Baldwin et al., Journal of Chemical Biology (2003) 10:1085-1094), loci of engineered recombinases such as Tre recombinase, Brec1 recombinase (Sarkar et al., Science (2007) 316:1912-1915; Karpinski et al., Nature Biotech (2016) 34:401-409) or engineered ZFNs (Sirk et al., Nucleic Acids Research (2014) 42:4755-66).

The gene fragment harboring the engineered recombination locus is then introduced into a selected cell line, such as into HEK293 cells, using techniques well known in the art, such as via nucleofection. Guide polynucleotides, such as but not limited to sgRNAs, are introduced into cells that constitutively express a Cas endonuclease, such as Cas9, or guide RNA/Cas complexes are introduced into cells that do not express a Cas endonuclease. The Cas endonuclease, such as Cas9, makes a double-strand break at a position within the AAVS1 locus that corresponds to the region of homology arms flanking the synthesized recombination locus fragment. The locus is then incorporated into the AAVS1 site via homology-directed DNA repair. Cells are passaged into single cell clones and the incorporation of the engineered locus is assessed by sequencing, such as by Next Generation Sequencing. Clonal cell populations that have incorporated the recombination locus into one of the two AAVS1 genomic loci (heterozygous incorporation) are suitable for use in recombinase mediated protein diversification. A number of expression vectors can be produced and introduced into this engineered protein diversification cell line.

For example, if structural data is unavailable for a protein of interest, the genomic sequence consisting of introns and exons can be computationally segregated into any number of gene fragments. As shown in FIG. 13, the genomic sequence can be divided to yield three fragments of approximately equal size. Note that the number of fragments here is not limited to three, but as described above, can vary. Gene fragment sequences are flanked by introns and donor recombination sites such that they can be inserted by recombinase activity between corresponding recombination acceptor sites in the engineered locus. The gene fragments are designed such that insertion into the recombination locus will retain the sequential order of gene fragments from the endogenous genomic sequence upon integration. Gene fragments can be synthesized (e.g., commercially) as double-stranded DNA and then cloned into a vector for delivery into cells by transfection.

In another embodiment, a DNA library for protein domain shuffling for a given protein when 3D structural information is available and for use with the engineered protein diversification cell line above, can be produced. For example, DNA from two or more protein homologues can be combined to produce a new protein chimera. Such rational design of a protein chimera is not limited to protein homologues and can be extended to protein orthologues and even unrelated protein domains. A target protein is selected, such as, but not limited to, a protein as described above. If a protein structure for the target protein is available the structure is used in the library design. If no structural data is available but the structure of a protein homologue is available, the homologous structure is used to build an approximate structural model of the target protein or sub-domains of interest, using a computer program, such as but not limited to, the program MODELLER).

The target protein sequence can be aligned with other homologous protein sequences in an alignment program (for example using ClustalO or Jalview). Using structural information, secondary structure predictions and the sequence alignment, the protein is computationally “cut” into segments. Criteria for suitable “cut sites” include but are not limited to: the beginning or end of domains or secondary structure elements, at the beginning or end of alpha helices, at the beginning or the end of loops, at the beginning or the end of beta strands. Prediction of contiguous (SCHEMA, Endelmann et al., Protein Engineering, Design & Selection (2004) 17: 589-594) or non-contiguous protein fragments (Smith et al., Protein Science (2013) 22:231-238) are known in the art. To enable downstream cleavage by a nuclease, such as Cas9, suitable PAM sites, such as an NGG motif for Cas9, that lead to silent mutations, can be engineered into the DNA fragments. DNA gene fragments designed via this method are then synthesized by a manufacturer (e.g. TWIST Biosciences, Agilent, Synthego (Redwood City, Calif.)), and cloned into a suitable vector for protein expression, further recombination, e.g., using the engineered protein diversification cell line above, or viral integration into a host genome (such as that of a mammalian, yeast or bacterial cell).

In another embodiment, a DNA library for protein family shuffling when 3D structural information is not available can be prepared and can combine DNA from two or more protein homologues into one new protein chimera entity. The library design and choice of restriction enzymes for family shuffling are known in the art (see. e.g., Crameri et al., Nature (1998) 391:288-291, reviewed in Huang et al., BioTechniques (2016) 60:91-94 2016). In short, the target protein and one or more homologues are chosen. To enable downstream cleavage by a nuclease (such as Cas9) suitable PAM sites (such as an NGG motif for Cas9) that lead to silent mutations can be engineered into the DNA. DNA gene fragments designed via this method are then synthesized by a manufacturer (e.g. TWIST Biosciences, Agilent, Synthego). The DNA sequences are fragmented into smaller DNA pieces of variable size with a suitable restriction enzyme (e.g. DNasel or EcoRI) and fragmented DNA pieces of two or more homologous sequences are then recombined using primerless PCR. The recombined chimeric DNA sequences can be cloned into a suitable vector for protein expression or further homologous recombination or viral integration into a host genome (such as mammalian cell, yeast cell, bacterial cell).

Any of the above expression vectors can be transfected into the engineered protein diversification cell line above, along with a recombinase expression vector, to drive site-specific integration of the DNA sequences therein. The coding sequences present in the vector can be codon optimized and are chosen based on the recombination acceptor sites present in the integrated artificial recombination cassette in the cell line. For example, if the recombination loci include FRT, LoxP and AttB sites, as described above, Flp, Cre and/or psi C31 are cloned into suitable expression vectors, such as lentiviral expression vectors, for expression in mammalian cells. Flp recombinase expression will drive recombination between FRT sites. Cre recombinase expression will drive recombination between LoxP sites and psi C31 recombinase will drive recombination between Att sites. Although the above discussion is with respect to Flp, Cre and psi C31 phage-derived recombinases, other known transposase or integrase enzymes in combination with alternate recognition sites can also be used.

Transfection of the various expression vectors can be accomplished by any of several techniques known in the art, including nucleofection, viral transduction, and the like. Recombinase expression then takes place and gene fragments undergo site-specific insertion from the gene fragment donor library. The engineered recombination locus is actively transcribed into an RNA molecule from the gene fragments inserted. RNA splicing removes introns including those in which the recombinase acceptor sites are nested. Gene fragments inserted will thus yield a mature RNA in which coding exons of each gene fragment are sequentially joined in the designated order. This mature RNA is translated into protein for assessment in downstream functional assays. The engineered recombination locus can be utilized in combination with expanded libraries of variants for each gene fragment. Stochastic integration of a variant from each gene fragment library with each fragment in an engineered 5′ to 3′ order defined by recombination sites, yields protein diversification with functional protein configuration.

As discussed above, the methods for generating diverse protein engineering libraries described herein make use of programmable endonucleases. In some embodiments, the programmable endonucleases used are derived from the CRISPR-Cas system. For each of the above-described embodiments, when Cas9 proteins are used, any of various Cas9-derived proteins can be used, as well as other CRISPR-Cas proteins as detailed above.

A number of catalytically active Cas9 proteins are known in the art and, as explained above, a Cas9 protein for use herein can be derived from any bacterial species, subspecies or strain that encodes the same. Although in certain embodiments herein, the methods are exemplified using S. pyogenes Cas9, orthologs from other bacterial species will find use herein. The specificity of these Cas9 orthologs is well known. Also useful are proteins encoded by Cas9-like synthetic proteins, and variants and modifications thereof. As explained above, the sequences for hundreds of Cas9 proteins are known and any of these proteins will find use with the present methods.

Additionally, it is to be understood that other Cas nucleases, in place of or in addition to Cas9, may be used, including any of the Cas proteins described in detail above, such as derived from any of the various CRISPR-Cas classes, types and subtypes.

Moreover, in the embodiments described herein, sgRNA is used as an exemplary guide polynucleotide, however, it will be recognized by one of skill in the art that other guide polynucleotides that site-specifically guide endonucleases, such as CRISPR-Cas proteins to a target nucleic acid can be used. The sgRNA component of the complexes is responsible for targeting a particular nucleic acid target. In particular, the spacer region of the sgRNA includes the region of complementarity to the targeted nucleic acid sequence. Thus, the spacer is the polynucleotide sequence that can specifically hybridize to a target nucleic acid sequence. The spacer element interacts with the target nucleic acid sequence through hydrogen bonding between complementary base pairs. A spacer element binds to a selected nucleic acid target sequence. Accordingly, the spacer element is the DNA target-binding sequence.

Thus, binding specificity is determined by both sgRNA-DNA base pairing and the PAM sequence juxtaposed to the DNA complementary region.

If CRISPR complexes are used, they can be produced using methods well known in the art. For example, guide RNA components of the complexes can be produced in vitro and Cas9 components can be recombinantly produced and then the two complexed together using methods known in the art. Additionally, cell lines such as but not limited to HEK293 cells, are commercially available that constitutively express S. pyogenes Cas9 as well as S. pyogenes Cas9-GFP fusions. In this instance, cells expressing Cas9 can be transfected with the guide RNA components and complexes are purified from the cells using standard purification techniques, such as but not limited to affinity, ion exchange and size exclusion chromatography. See, e.g., Jinek M., et al., “A programmable dual-RNA-guided DNA endonuclease in adaptive bacterial immunity,” Science (2012) 337:816-821.

More than one set of complexes can be used, such as 2-50 or more, for example 5-20, 8-15, etc., or any number within these ranges.

The complexes, such as sgRNA/Cas9 complexes may be introduced to cells at differing concentrations. For example, sgRNA/Cas9 complexes can be introduced at a ratio of 1:2, 1:3, 1:4, 1:5, 1:6, 1:7, 1:8, 1:9, 1:10, 10:1, 9:1, 8:1, 7:1, 6:1, 5:1, 4:1, 3:1, or 2:1. Additionally, all of these components, i.e., sgRNA and Cas9, may be provided separately, e.g., as separately assembled complexes, using separate DNA or RNA constructs, or together, in a single construct, or in any combination.

sgRNA/Cas9 complexes may be introduced at differing time points. For example, sgRNA/Cas9 complexes can be introduced at least 1 minute apart, 5 minutes apart, 10 minutes apart, 30 minutes apart, 1 hour apart, 5 hours apart, or 15 hours apart or more. sgRNA/Cas9 complexes can be introduced at most 1 minute apart, 5 minutes apart, 10 minutes apart, 30 minutes apart, 1 hour apart, 5 hours apart, or 15 hours apart or more. One set of complexes can be purified out before introducing another set of complexes. sgRNA/Cas9 complexes may be differentially regulated (i.e. differentially expressed or stabilized) via exogenously supplied agents (e.g. inducible DNA promoters or inducible Cas9 proteins).

Thus, in exemplary embodiments as described above, a sgRNA, complexed with Cas9 (sgRNA/Cas9 complex) is directed to a genomic locus of interest to induce double-strand breaks. The binding specificity is determined by both sgRNA-DNA base pairing and the PAM sequence juxtaposed to the DNA complementary region.

In all of the embodiments of the above-described methods, the various components can be produced by synthesis, or for example, using expression cassettes encoding a programmable endonuclease, such as a Cas protein, guide polynucleotide, etc. These components can be present on a single cassette or multiple cassettes, in the same or different constructs. Expression cassettes typically comprise regulatory sequences that are involved in one or more of the following: regulation of transcription, post-transcriptional regulation, and regulation of translation. Expression cassettes can be introduced into a wide variety of organisms including bacterial cells, yeast cells, plant cells, and mammalian cells. Expression cassettes typically comprise functional regulatory sequences corresponding to the organism(s) into which they are being introduced.

In one aspect, all or a portion of the various components for use in the methods are produced in vectors, including expression vectors, comprising polynucleotides encoding therefor. Vectors useful for producing components for use in the present methods include plasmids, viruses (including phage), and Integratable DNA fragments (i.e., fragments integratable into the host genome by homologous recombination). A vector replicates and functions independently of the host genome, or may, in some instances, integrate into the genome itself. Suitable replicating vectors will contain a replicon and control sequences derived from species compatible with the intended expression host cell. Transformed host cells are cells that have been transformed or transfected with the vectors constructed using recombinant DNA techniques

General methods for construction of expression vectors are known in the art. Expression vectors for most host cells are commercially available. There are several commercial software products designed to facilitate selection of appropriate vectors and construction thereof, such as insect cell vectors for insect cell transformation and gene expression in insect cells, bacterial plasmids for bacterial transformation and gene expression in bacterial cells, yeast plasmids for cell transformation and gene expression in yeast and other fungi, mammalian vectors for mammalian cell transformation and gene expression in mammalian cells or mammals, viral vectors (including retroviral, lentiviral, and adenoviral vectors) for cell transformation and gene expression and methods to easily enable cloning of such polynucleotides. SnapGene™ (GSL Biotech LLC, Chicago, Ill.;

snapgene.com/resources/plasmid_files/your_time_is_valuable/), for example, provides an extensive list of vectors, individual vector sequences, and vector maps, as well as commercial sources for many of the vectors.

Expression cassettes typically comprise regulatory sequences that are involved in one or more of the following: regulation of transcription, post-transcriptional regulation, and regulation of translation. Expression cassettes can be introduced into a wide variety of organisms including bacterial cells, yeast cells, mammalian cells, and plant cells. Expression cassettes typically comprise functional regulatory sequences corresponding to the host cells or organism(s) into which they are being introduced. Expression vectors can also include polynucleotides encoding protein tags (e.g., poly-His tags, hemagglutinin tags, fluorescent protein tags, bioluminescent tags, nuclear localization tags). The coding sequences for such protein tags can be fused to the coding sequences or can be included in an expression cassette, for example, in a targeting vector.

In some embodiments, polynucleotides encoding one or more of the various components are operably linked to an inducible promoter, a repressible promoter, or a constitutive promoter.

Several expression vectors have been designed for expressing guide polynucleotides. See, e.g., Shen, B. et al. “Efficient genome modification by CRISPR-Cas9 nickase with minimal off-target effects” (2014) Mar. 2. doi: 10.1038/nmeth.2857. 10.1038/nmeth.2857. Additionally, vectors and expression systems are commercially available, such as from New England Biolabs (Ipswich, Mass.) and Clontech Laboratories (Mountain View, Calif.). Vectors can be designed to simultaneously express a target-specific sgRNA using a U2 or U6 promoter, a Cas9 and/or dCas9, and if desired, a marker protein, for monitoring transfection efficiency and/or for further enriching/isolating transfected cells by flow cytometry.

Vectors can be designed for expression of various components of the described methods in prokaryotic or eukaryotic cells. Alternatively, transcription can be in vitro, for example using T7 promoter regulatory sequences and T7 polymerase. Other RNA polymerase and promoter sequences can be used.

Vectors can be introduced into and propagated in a prokaryote. Prokaryotic vectors are well known in the art. Typically a prokaryotic vector comprises an origin of replication suitable for the target host cell (e.g., oriC derived from E. coli, pUC derived from pBR322, pSC101 derived from Salmonella), 15A origin (derived from p15A) and bacterial artificial chromosomes). Vectors can include a selectable marker (e.g., genes encoding resistance for ampicillin, chloramphenicol, gentamicin, and kanamycin). Zeocin™ (Life Technologies, Grand Island, N.Y.) can be used as a selection in bacteria, fungi (including yeast), plants and mammalian cell lines. Accordingly, vectors can be designed that carry only one drug resistance gene for Zeocin for selection work in a number of organisms. Useful promoters are known for expression of proteins in prokaryotes, for example, T5, T7, Rhamnose (inducible), Arabinose (inducible), and PhoA (inducible). Further, T7 promoters are widely used in vectors that also encode the T7 RNA polymerase. Prokaryotic vectors can also include ribosome binding sites of varying strength, and secretion signals (e.g., mal, sec, tat, ompC, and pelB). In addition, vectors can comprise RNA polymerase promoters for the expression of sgRNAs. Prokaryotic RNA polymerase transcription termination sequences are also well known (e.g., transcription termination sequences from S. pyogenes).

Integrating vectors for stable transformation of prokaryotes are also known in the art (see, e.g., Heap, J. T., et al., “Integration of DNA into bacterial chromosomes from plasmids without a counter-selection marker,” Nucleic Acids Res. (2012) 40:e59).

Expression of proteins in prokaryotes is typically carried out in Escherichia coli with vectors containing constitutive or inducible promoters directing the expression of either fusion or non-fusion proteins.

A wide variety of RNA polymerase promoters suitable for expression of the various components are available in prokaryotes (see, e.g., Jiang, Y., et al., “Multigene editing in the Escherichia coli genome via the CRISPR-Cas9 system,” Environ Microbiol. (2015) 81:2506-2514); Estrem, S. T., et al., (1999) “Bacterial promoter architecture: subsite structure of UP elements and interactions with the carboxy-terminal domain of the RNA polymerase alpha subunit,” Genes Dev. 15; 13(16):2134-47).

In some embodiments, a vector is a yeast expression vector comprising one or more components of the above-described methods. Examples of vectors for expression in Saccharomyces cerivisae include, but are not limited to, the following: pYepSec1, pMFa, pJRY88, pYES2, and picZ. Methods for gene expression in yeast cells are known in the art (see, e.g., Methods in Enzymology, Volume 194, “Guide to Yeast Genetics and Molecular and Cell Biology, Part A,” (2004) Christine Guthrie and Gerald R. Fink (eds.), Elsevier Academic Press, San Diego, Calif.). Typically, expression of protein-encoding genes in yeast requires a promoter operably linked to a coding region of interest plus a transcriptional terminator. Various yeast promoters can be used to construct expression cassettes for expression of genes in yeast. Examples of promoters include, but are not limited to, promoters of genes encoding the following yeast proteins: alcohol dehydrogenase 1 (ADH1) or alcohol dehydrogenase 2 (ADH2), phosphoglycerate kinase (PGK), triose phosphate isomerase (TPI), glyceraldehyde-3-phosphate dehydrogenase (GAPDH; also known as TDH3, or triose phosphate dehydrogenase), galactose-1-phosphate uridyl-transferase (GAL7), UDP-galactose epimerase (GAL10), cytochrome ci (CYC1), acid phosphatase (PHOS) and glycerol-3-phosphate dehydrogenase gene (GPD1). Hybrid promoters, such as the ADH2/GAPDH, CYC1/GAL10 and the ADH2/GAPDH promoter (which is induced at low cellular-glucose concentrations, e.g., about 0.1 percent to about 0.2 percent) also may be used. In S. pombe, suitable promoters include the thiamine-repressed nmt1 promoter and the constitutive cytomegalovirus promoter in pTL2M.

Yeast RNA polymerase III promoters (e.g., promoters from 5S, U6 or RPR1 genes) as well as polymerase III termination sequences are known in the art (see, e.g., yeastgenome.org; Harismendy, O., et al., (2003) “Genome-wide location of yeast RNA polymerase III transcription machinery,” The EMBO Journal. 22(18):4738-4747.)

In addition to a promoter, several upstream activation sequences (UASs), also called enhancers, may be used to enhance polypeptide expression. Exemplary upstream activation sequences for expression in yeast include the UASs of genes encoding these proteins: CYC1, ADH2, GAL1, GAL7, GAL10, and ADH2. Exemplary transcription termination sequences for expression in yeast include the termination sequences of the α-factor, CYC1, GAPDH, and PGK genes. One or multiple termination sequences can be used.

Suitable promoters, terminators, and coding regions may be cloned into E. coli-yeast shuttle vectors and transformed into yeast cells. These vectors allow strain propagation in both yeast and E. coli strains. Typically, the vector contains a selectable marker and sequences enabling autonomous replication or chromosomal integration in each host. Examples of plasmids typically used in yeast are the shuttle vectors pRS423, pRS424, pRS425, and pRS426 (American Type Culture Collection, Manassas, Va.). These plasmids contain a yeast 2 micron origin of replication, an E. coli replication origin (e.g., pMB1), and a selectable marker.

The various components can also be expressed in insects or insect cells. Suitable expression control sequences for use in such cells are well known in the art. In some embodiments, it is desirable that the expression control sequence comprises a constitutive promoter. Examples of suitable strong promoters include, but are not limited to, the following: the baculovirus promoters for the piO, polyhedrin (polh), p 6.9, capsid, UAS (contains a Gal4 binding site), Ac5, cathepsin-like genes, the B. mori actin gene promoter; Drosophila melanogaster hsp70, actin, α-1-tubulin or ubiquitin gene promoters, RSV or MMTV promoters, copia promoter, gypsy promoter, and the cytomegalovirus IE gene promoter. Examples of weak promoters that can be used include, but are not limited to, the following: the baculovirus promoters for the ie1, ie2, ieO, etl, 39K (aka pp31), and gp64 genes. If it is desired to increase the amount of gene expression from a weak promoter, enhancer elements, such as the baculovirus enhancer element, hr5, may be used in conjunction with the promoter.

For the expression of some of the components of the present invention in insects, RNA polymerase III promoters are known in the art, for example, the U6 promoter. Conserved features of RNA polymerase III promoters in insects are also known (see, e.g., Hernandez, G., (2007) “Insect small nuclear RNA gene promoters evolve rapidly yet retain conserved features involved in determining promoter activity and RNA polymerase specificity,” Nucleic Acids Res. 2007 January; 35(1):21-34).

In another aspect, the various components are incorporated into mammalian vectors for use in mammalian cells. A large number of mammalian vectors suitable for use with the systems of the present invention are commercially available (e.g., from Life Technologies, Grand Island, N.Y.; NeoBiolab, Cambridge, Mass.; Promega, Madison, Wis.; DNA2.0, Menlo Park, Calif.; Addgene, Cambridge, Mass.).

Vectors derived from mammalian viruses can also be used for expressing the various components of the present methods in mammalian cells. These include vectors derived from viruses such as adenovirus, papovirus, herpesvirus, polyomavirus, cytomegalovirus, lentivirus, retrovirus, vaccinia and Simian Virus 40 (SV40) (see, e.g., Kaufman, R. J., (2000) “Overview of vector design for mammalian gene expression,” Molecular Biotechnology, Volume 16, Issue 2, pp 151-160; Cooray S., et al., (2012) “Retrovirus and lentivirus vector design and methods of cell conditioning,” Methods Enzymol. 507:29-57). Regulatory sequences operably linked to the components can include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like. Commonly used promoters are constitutive mammalian promoters CMV, EF1a, SV40, PGK1 (mouse or human), Ubc, CAG, CaMKIIa, and beta-Act, and others known in the art (Khan, K. H. (2013) “Gene Expression in Mammalian Cells and its Applications,” Advanced Pharmaceutical Bulletin 3(2), 257-263). Further, mammalian RNA polymerase III promoters, including H1 and U6, can be used.

In some embodiments, a recombinant mammalian expression vector is capable of preferentially directing expression of the nucleic acid in a particular cell type (e.g., using tissue-specific regulatory elements to express a polynucleotide). Tissue-specific regulatory elements are known in the art and include, but are not limited to, the albumin promoter, lymphoid-specific promoters, neuron-specific promoters (e.g., the neurofilament promoter), pancreas-specific promoters, mammary gland-specific promoters (e.g., milk whey promoter), and in particular promoters of T cell receptors and immunoglobulins. Developmentally-regulated promoters are also encompassed, e.g., the murine hox promoters and the alpha-fetoprotein promoter.

Numerous mammalian cell lines have been utilized for expression of gene products including HEK 293 (Human embryonic kidney) Jurkat cells (an immortalized line of human T lymphocyte cells) and CHO (Chinese hamster ovary). These cell lines can be transfected by standard methods (e.g., using calcium phosphate or polyethyleneimine (PEI), or electroporation). Other typical mammalian cell lines include, but are not limited to: HeLa, U2OS, 549, HT1080, CAD, P19, NIH 3T3, L929, N2a, Human embryonic kidney 293 cells, MCF-7, Y79, SO-Rb50, Hep G2, DUKX-X11, J558L, and Baby hamster kidney (BHK) cells.

Methods of introducing polynucleotides (e.g., an expression vector) into host cells are known in the art and are typically selected based on the kind of host cell. Such methods include, for example, viral or bacteriophage infection, transfection, conjugation, electroporation, calcium phosphate precipitation, polyethyleneimine-mediated transfection, DEAE-dextran mediated transfection, protoplast fusion, lipofection, liposome-mediated transfection, particle gun technology, direct microinjection, and nanoparticle-mediated delivery.

While preferred embodiments of the present invention have been shown and described herein, it will be obvious to those skilled in the art that such embodiments are provided by way of example only. From the above description and the following Examples, one skilled in the art can ascertain essential characteristics of this invention, and without departing from the spirit and scope thereof, can make changes, substitutions, variations, and modifications of the invention to adapt it to various usages and conditions. Such changes, substitutions, variations, and modifications are also intended to fall within the scope of the present disclosure.

### EXPERIMENTAL

Aspects of the present invention are further illustrated in the following Examples. Efforts have been made to ensure accuracy with respect to numbers used (e.g., amounts, concentrations, percent changes, etc.) but some experimental errors and deviations should be accounted for. Unless indicated otherwise, temperature is in degrees Centigrade and pressure is at or near atmospheric. It should be understood that these Examples, while indicating some embodiments of the invention, are given by way of illustration only.

The following Examples are not intended to limit the scope of what the inventors regard as various aspects of the present invention.

### Example 1

### Characterization of DNA Repair Patterns for Use in Generating Molecular Libraries

As detailed below, DNA repair patterns in human cells can be manipulated and the DNA repair pathway initiated after the creation of double-strand breaks (DSBs) and can be used to generate protein libraries.

### A. DNA Repair of DSBs is Non-Random:

In order to assess DNA repair outcomes in human cells, the following experiment was conducted using methods as described in van Overbeek et al., Molecular Cell (2016) http://dx.doi.org/10.1016/j.molcel.2016.06.037. Briefly, the repair outcomes following Cas9 cleavage of double-stranded DNA, which result in blunt end products, were profiled using computational tools developed to categorize indels from a cell-based assay. To this end, HEK293, K562 and CCRF-CEM cells were transfected with pre-assembled complexes of Cas9 protein and sgRNA (single-guide RNA ribonucleoprotein complexes (sgRNPs). The Cas9/sgRNA complexes used were prepared as described in van Overbeek et al., Molecular Cell (2016) http://dx.doi.org/10.1016/j.molcel.2016.06.037. DNA repair patterns following Cas9 cleavage were analyzed by amplicon sequencing at time points as shown in FIGS. 6A-6F.

Sequencing reads were assigned to a specific indel class based on the indel type (insertion or deletion), start site, and length (or to the wild-type class), and then the frequency of each class was calculated as a fraction of aligned reads or as a fraction of mutant reads.

Results are shown in FIGS. 6A-6F which depict the top five repair classes and wild-type repair outcomes (classes and frequency) as monitored by amplicon sequencing 48 hours after nucleofection of sgRNP in HEK293 (FIG. 6A); 14 days after constitutive expression of sgRNA and Cas9 in HEK293T (FIG. 6B); 48 hours after nucleofection of sgRNP in K562 (FIG. 6C); 48 hours after nucleofection of sgRNP in donor derived T cells (FIG. 6D); 48 hours after nucleofection of sgRNP in CCRF-CEM (FIG. 6E); and 48 hours after nucleofection of sgRNP in HEK293 plus DNAPK inhibitor NU7441 (FIG. 6F). The arrows from FIG. 6A to FIGS. 6B, 6C and 6D indicate similar DNA repair outcomes compared with FIG. 6A. The arrows from FIG. 6A to FIGS. 6E and 6F indicate different DNA repair outcomes compared with FIG. 6A.

Large scale computational analyses of double-strand breaks indicated the same DNA repair patterns were observed across experimental replicates, cell lines and reagent delivery methods.

### B. Compounds can Alter DNA Repair Pathways:

In order to assess whether compounds can alter the DNA repair pathways, the following experiment was conducted with methods as described in van Overbeek et al., Molecular Cell (2016) http://dx.doi.org/10.1016/j.molcel.2016.06.037. Briefly, to test the hypothesis that suppression of c-NHEJ would alter the DNA repair landscapes, favoring MMEJ repair outcomes, a chemical inhibitor of DNA-PK, NU7441, was added to HEK293 T cells one hour post nucleofection in a five point dose response. Genomic lysate was harvested 48 hours post nucleofection and processed.

At the lowest concentration of inhibitor (1.56 μM), a change of the DNA repair patterns at 12 different target sites compared with untreated samples was apparent. As inhibitor concentration increased, the mean frequency of single base insertions and small deletions (<3 base pairs) decreased, whereas the mean frequency of a subset of large deletions (>3 base pairs) present in the DNA repair profiles increased. This experiment evidences that the suppression of c-NHEJ enhances DNA repair by MMEJ pathways after DSB formation by Cas9. These data also indicate that DNA editing events produced by different DNA repair machineries by DNA repair profiling can be effectively segregated and that individual repair profiles can be modulated by suppressing or enhancing individual components of these pathways.

Accordingly, chemically altering DNA repair pathways perturbs the conserved DNA repair patterns.

C. Lymphoblastic Cell Lines have an Insertional Bias in DNA Repair:

In order to assess insertion-biased repair in human cells, the following experiment was conducted. Jurkat and CCRF-CEM are human lymphoblast cell lines and display altered DNA repair patterns compared to other cell lines, including primary T cells, to which they are most similar. Jurkat and CCRF-CEM cells were nucleofected with sgRNP complexes as in van Overbeek et al., Molecular Cell (2016) http://dx.doi org/10.1016/j.molcel.2016.06.037. For nucleofection, cells were resuspended in SE buffer and nucleofected with AMAXA NUCLEOFECTOR PROGRAM CL-120. 48 hours after nucleofection, genomic DNA was harvested with QUICKEXTRACT as described in van Overbeek et al., Molecular Cell (2016) http://dx.doi.org/10.1016/j.molcel.2016.06.037. Lysates were sequenced and results were aligned and analyzed as described in van Overbeek et al., Molecular Cell (2016) http://dx.doi.org/10.1016/j.molcel.2016.06.037. The top 15 indel classes for the JAK1 spacer are shown, demonstrating a bias towards small insertions in Jurkat and CCRF-CEM cells (FIGS. 6 and 7). FIGS. 7A-7B depict repair outcomes at LINC00441 target site (chr13:48303392-48303414 (hg38)). The top fifteen repair classes and wild-type are depicted in each repair browser view. FIG. 7A shows DNA repair outcomes (classes and frequency) as monitored by amplicon sequencing 48 hours after nucleofection of sgRNP in HEK293 and FIG. 7B shows DNA repair outcomes (classes and frequency) as monitored by amplicon sequencing 48 hours after nucleofection of sgRNP in Jurkat cell lines.

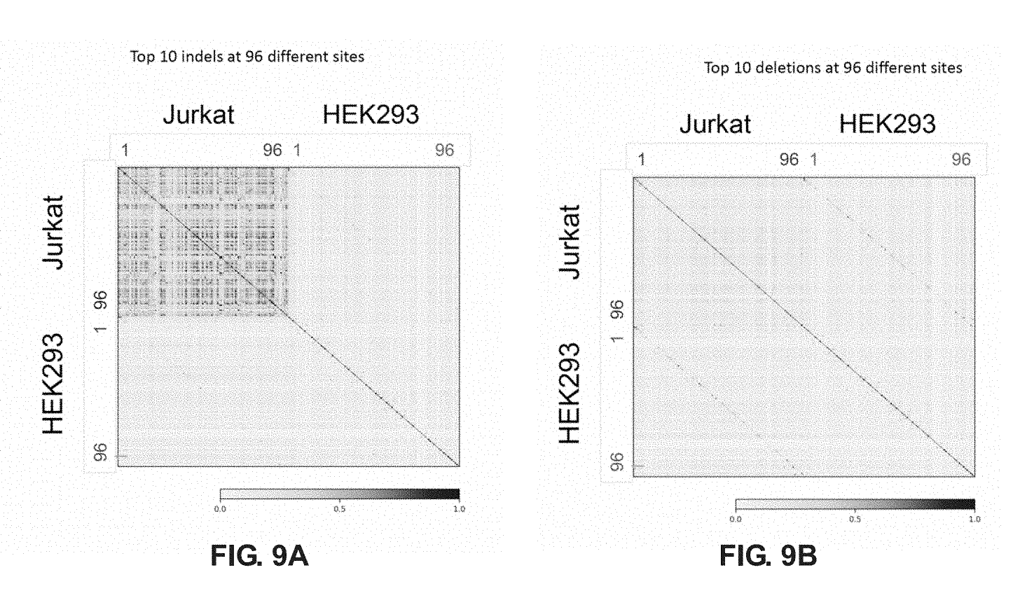

In order to analyze the DNA repair pattern in these cells in more detail, the Jaccard/Tanimoto coefficient for the top 10 indel repair classes at 96 sites in Jurkat and HEK293 cells was calculated (FIGS. 9A and 9B). The Jaccard/Tanimoto coefficient is a measure of the overlap in two sets of repairs (Jaccard, P., New Phytologist (1912)11:37-50; Rogers and Tanimoto, Science (1960) 132:1115-1118). A value of 1 indicates complete overlap in the two sets; a value of 0 indicates no overlap in the two sets. In FIG. 9A, when all repair classes were compared, there was no overlap between Jurkat and HEK293 cells. In FIG. 9B, when only deletions were analyzed, there was high overlap in DNA repair in Jurkat cells and HEK293 cells, indicating that DNA repair in Jurkat cells was biased towards insertions.

### D. Use of Exogenous DNA Results in Insertional Bias:

In order to assess insertion-biased repair using an exogenous DNA, the following experiment was conducted. HEK293 cells constitutively expressing Cas9 were transfected with 300 ng sgRNA and 200 ng either pUC19 plasmid DNA, Herring Sperm DNA or random short oligos, using the methods described in van Overbeek et al., Molecular Cell (2016), http://dx.doi.org/10.1016/j.molcel.2016.06.037. Genomic DNA lysates were generated as described in van Overbeek et al. and treated with ExoI exonuclease for 30 minutes at 37C followed by heat inactivation at 80C for 20 minutes. Lysates were then sequenced and analyzed as before. Results are shown in FIGS. 8A-8C that depict repair outcomes at BRCA1 target site (chr17:43125332-43125354 (hg38). The top fifteen repair classes and wild-type are depicted. DNA repair outcomes (classes and frequency) as monitored by amplicon sequencing 48 hours after lipofection of sgRNA (FIG. 8A); sgRNA and herring sperm DNA (200 ng) (FIG. 8B); and sgRNA and a random DNA oligo pool in a HEK293 Cas9-GFP expressing cell line (FIG. 8C). Analysis of DNA repair indicated a shift in the pattern toward small insertions when Herring Sperm and short oligos but not pUC19 plasmid DNA were used (FIG. 8).

Thus, introduction of exogenous DNA during the transfection of CRISPR reagents results in an insertional bias in the resulting DNA repair.

### E. Estimation of Combinatorial Diversity:

Insertional bias in DNA repair results in many more repair classes than the typical DNA repair pattern at a given site. Each of these repair classes represents a unique gene sequence that could result in a protein with novel function. Introducing this type of diversity at multiple sites within a gene can allow for the rapid generation of dynamic DNA libraries in cells.

In order to estimate the potential size of these libraries, four sgRNPs targeted to the T cell receptor beta variable 9 (TRBV9) gene were individually nucleofected in Jurkat cells using methods previously described. The resultant DNA repair patterns were analyzed and for each target, the number of unique, in-frame gene sequences were counted. The in-frame sequences were analyzed for the frequency of STOP codon insertions and this number was subtracted from the number of unique, in-frame sequences.

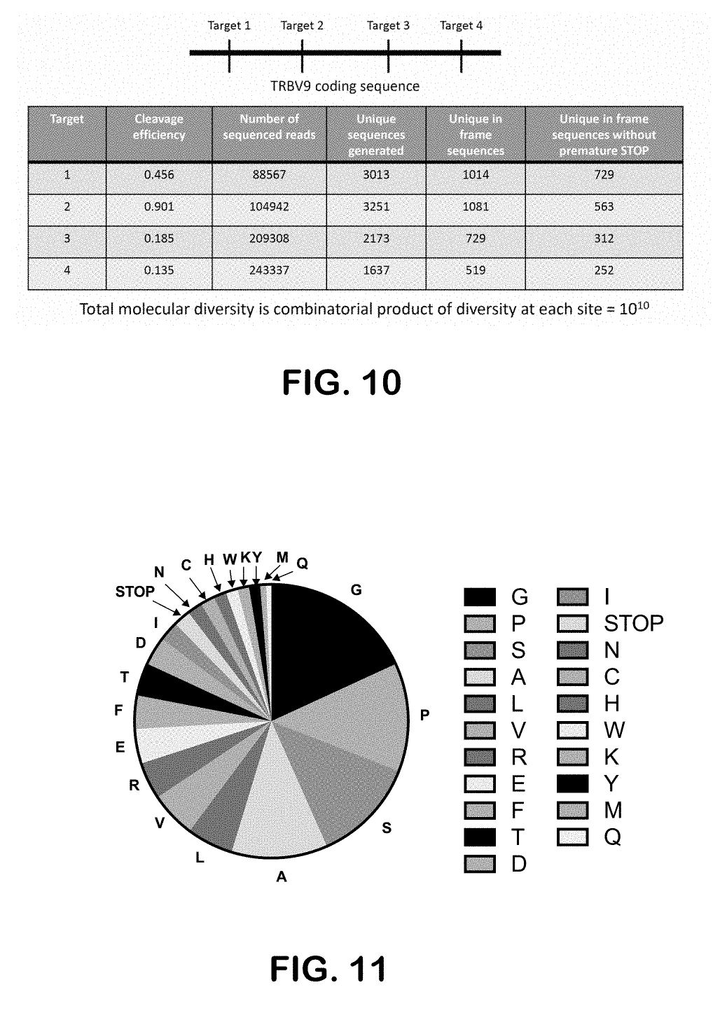

To estimate the combinatorial library size resulting from iterative editing at all four sites, the number of possible sequences for each site were multiplied. As shown in FIG. 10, this yielded 10¹⁰ possible unique, in frame gene sequences, resulting from editing at just four sites in this gene.

The sequences of these in-frame insertions were analyzed computationally to determine which amino acids were encoded. As shown in FIG. 11, this analysis revealed that all 20 amino acids were inserted.

### Example 2

### Use of Insertion-Biased DNA Repair to Create Molecular Libraries for Discovery of Proteins with Novel Functions

This Example describes how insertion-biased DNA repair resulting from Cas9-mediated DSBs can be used to create dynamic molecular libraries for the discovery of proteins with novel function.

### A. Use of Lymphoblast Cell Lines to Create Libraries of an Endogenous Gene:

This Example describes methods that are used to create molecular libraries of endogenously expressed genes in lymphoblastic cell lines that display insertional bias in their DNA repair patters, such as but not limited to Jurkat and CCRF-CEM cell lines. Combined with appropriate screening methods, these libraries are used to identify novel gene sequences encoding for proteins with desirable functions.

For example, Jurkat cells have high expression levels of Vβ8, a T cell receptor subunit that mediates binding to staphylococcal enterotoxin B (SEB). Native Vβ8-SEB binding is low affinity. In order to engineer Vβ8 proteins with higher affinity for e.g., therapeutic use, sgRNAs are designed to the desired regions of the Vβ8 genes. These sgRNAs are delivered to cells in RNP complexes with Cas9 protein. The edited cells are screened by an SEB binding assay using flow cytometry as in Sharma et al, Protein Engineering, Design & Selection (2013) 26: 781-789, where binding of biotinylated SEB is detected used fluorophore-conjugated streptavidin reagents. In order to select for Vβ8 proteins with increased binding affinity for SEB, the stringency of the binding assay is increased in each round by decreasing the concentration of SEB used.

In one embodiment of this procedure, populations of cells that show some binding activity are sorted after each round of editing, expanded and re-transfected for iterative rounds of editing. In another embodiment of this procedure, cells are re-transfected and edited multiple times before screening at the population level. When a population reaches a desired level of binding activity, individual clones are sorted, expanded and sequenced to recover the mutations that result in enhanced function.

### B. Use of Lymphoblast Cell Lines to Create Libraries of an Exogenous Gene:

This Example describes the methods that are used to create molecular libraries of exogenously expressed genes in lymphoblastic cell lines that display insertional bias in their DNA repair patters, such as but not limited to Jurkat and CCRF-CEM cell lines. Combined with appropriate screening methods, these libraries are used to identify novel gene sequences encoding for proteins with desirable functions.

For example, a cassette encoding wild-type Aequorea victoria Green Fluorescent Protein (GFP) fused to a 2A self-cleaving peptide and a blasticidin resistance sequence and flanked by homology arms, is inserted into the Adeno-Associated Virus Integration Site 1 (AAVS1) locus using Cas9-mediated homology directed repair in Jurkat cells (FIG. 14A). After selection and verification of the insert, single cell clones are expanded to create a stable cell line expressing the GFP at the desired locus. sgRNAs targeting the GFP coding sequence are designed and electroporated into the cells as an RNP complex with Cas9 protein. The cells are grown with blasticidin to select for sequences with in-frame insertions in GFP. Cells are sorted using FACS to isolate those with enhanced green fluorescence. These cells are expanded and then re-transfected with guides to introduce further diversity (FIG. 14B). This process is repeated iteratively until a population with the desired fluorescent properties is obtained. Single cell clones are sorted from this population, expanded and sequenced to recover the mutations that resulted in enhanced function.

### C. Use of Exogenous DNA to Create Libraries of an Endogenous Gene:

This Example describes the methods that are used to create molecular libraries of endogenously expressed genes in any cell line or primary cell type using the insertional bias in DNA repair created by the delivery of exogenous DNA when introducing DSBs. Combined with appropriate screening methods, these libraries are used to identify novel gene sequences encoding for proteins with desirable functions.

Targeted DSBs are introduced using any number of methods known in the art, including but not limited to Cas9-mediated breaks introduced by delivery of sgRNAs into cells constitutively expressing Cas9, or delivery of sgRNP/Cas9 complexes. Herring Sperm DNA or random oligos of length including but not limited to N5, N6, N9, N10, N15 and N20 are concurrently transfected into cells, resulting in an insertional bias in DNA repair at the cut site directed by the sgRNA, as detailed in FIGS. 7 and 8.

In one embodiment, the sgRNAs are targeted to endogenously expressed genes in any cell line, including but not limited to immunoglobulin genes, T cell receptors, cytokine receptors, cell adhesion molecules, G-protein coupled receptors or enzymes. Combined with an appropriate screen such as a binding assay, cells expressing mutated genes coding for proteins with the desired function can be separated, cloned and the sequence isolated.

### D. Use of Exogenous DNA to Create Libraries of an Exogenous Gene:

This Example describes the methods that are used to create molecular libraries of exogenously expressed genes in any cell line or primary cell type using the insertional bias in DNA repair created by the delivery of exogenous DNA when introducing DSBs. Combined with appropriate screening methods, these libraries are used to identify novel gene sequences encoding for proteins with desirable functions.

Targeted DSBs are introduced using any number of methods known in the art, including but not limited to Cas9-mediated breaks introduced by delivery of sgRNAs into cells constitutively expressing Cas9, or delivery of sgRNP-Cas9 complexes. Herring Sperm DNA or random oligos of length including but not limited to N5, N6, N9, N10, N15 and N20 are concurrently transfected into cells, resulting in an insertional bias in DNA repair at the cut site directed by the sgRNA, as detailed in FIGS. 7 and 8.

In one embodiment, a cassette encoding wild-type recombinases such as from the Cre family, transposases or programmable nucleases, such as Cas9, is fused to a 2A self-cleaving peptide, and a blasticidin-resistance sequence flanked by homology arms is inserted into the AAVS1 locus using Cas9-mediated homology-directed repair in any cell line or primary cell type as in Example 2B. After selection and verification of the insert, single cell clones are expanded to create a stable cell line expressing the cassette at the desired locus. The sgRNAs are designed to target the entire coding region of this protein of interest, or specific regions predicted to be involved in the function of the protein. Combined with an appropriate screen such as a recombination or DNA cleavage assay, cells expressing mutated genes coding for proteins with the desired function can be separated, cloned and the sequence isolated.

In the Cre recombinase embodiment described above, molecular libraries of the protein are screened for mutants that can recombine novel DNA sequences. These novel recombination sequences could be unique sequences within safe harbor genes such as AAVSI. The screen entails transfecting the cells with a plasmid encoding a fluorescent marker such as GFP and a stop codon flanked by recombinase recognition sites, similar to the Substrate Linked Protein Evolution system used in Buchholz et al., Nature Biotechnology (2001) 19:1047-1052. A mammalian promoter is immediately upstream of the 5′ recombinase recognition site and a different fluorescent protein, such as mCherry, is immediately downstream of the 3′ recombinase recognition site. All cells that express the plasmid will emit green fluorescence. If a mutant recombinase protein can recognize the given DNA sequences, the GFP gene is excised and the cells are no longer green. Such recombination will also result in expression of the second, red fluorescent protein, thereby creating a system in which the ratio of green and red fluorescent cells allows for the calculation of recombination efficiency.

### Example 3

### Site-Directed Base Substitution Systems

This Example illustrates methods for evolving/engineering a protein of interest while maintaining reading frame through DNA base substitution in the open reading frame of the protein of interest. The methods described below can be used to generate diversity within any given stretch of DNA sequence in an orthogonal manner to insertion-biased repair diversity or protein shuffling as described in other Examples.

Base substitution activity by a cytidine deaminase is regulated in mature B-cells and can occur in a dysregulated manner in a phenomena termed kataegis (D'Antonio et al., Cell Reports (2016) 16:672-683; Casellas et al., Nat Rev Immunol (2016) 16:164-176). The B cell specific activation-induced cytidine deaminase (AID) has been used in vitro to mature and expand the diversity of antibodies in mammalian display systems through base substitution (Bowers et al., Proc. Natl. Acad. Sci. USA (2011) 108:20455-20460; Bowers et al., Journal of Biological Chemistry (2014) 289:33557-33567).

A protein with site-specific base-substitution activity has been previously engineered (Komor et al, Nature (2016) 533:420-424; Nishida et al., Science (2016) 10.1126/science.aaf8729). 4×10⁷ protein variations with similar activity as described in Komor et al. are engineered containing three modules where Module 1 has base substitution capabilities such as deaminase activity (Knisbacher et al., Trends in Genetics (2016) 32:16-28); Module 2 has DNA site-directed capabilities such as SpyCas9; and Module 3 has modulators of DNA repair activities such as inhibitor of uracil-DNA glycosylase or BER (base-excision repair).

See, FIG. 12. A subset of these engineered proteins are screened for site-directed base substitution activity and candidates used to further evolve and diversify the Cre recombinase, or other protein of interest as described in Example 4, through site-directed DNA base substitution activity.

The modular proteins with site-directed deaminase activity are used in biochemical or cell-based systems to evolve and diversify proteins of interest. In a cell-based system, the cells can be engineered to modulate the outcomes of cytosine deaminase activity. Cells are engineered to abrogate expression of proteins that reverse the cytosine to uracil activity of the deaminase module, Module 1. For example, the enzyme uracil DNA glycosylase converts the uracil produced by deaminase activity back to the parental cytosine thus suppressing base substitution. To suppress uracil DNA glycosylase activity, the genes encoding proteins with uracil DNA glycosylase activity, such as UNG in human cells, are disrupted to yield a functional knock-out. To further enhance base substitution activity in a cell-based system, cells are engineered to promote mismatch repair DNA repair activity (MMR). This is achieved by increasing the expression of MMR factors such as the human protein product of the gene PMS2.

### Example 4

### Recombinase-Mediated Protein Diversification

This Example illustrates methods for engineering a cell line of interest to harbor an artificial recombination locus. The engineered locus supports the integration of protein modules (gene fragments) to yield diverse protein products for use in downstream assays in a manner termed “recombinase mediated protein diversification.” This Example depicts but is not limited to a recombination locus capable of accepting three gene fragments.

### A. Preparation of an Engineered Recombination Locus in a Cell Line of Interest:

The engineered recombination locus is first synthesized (e.g., commercially) as a double-stranded DNA fragment with 5′ and 3′ flanking homology arms for insertion into the Adeno-Associated Virus Integration Site 1 (AAVS1) locus of a cell line of interest, for example in HEK293 cells. The engineered recombination locus fragment consists of a 5′ AAVS1 homology region followed by a promoter sequence to drive transcription through the three downstream recombination regions. Here, a pair of FRT sites are immediately downstream of the promoter followed by paired LoxP sites, followed by paired AttB sites.

The FRT recognition site consists of the minimal 34 base sequence, GAAGTTCCTATTCtctagaaaGtATAGGAACTTC (SEQ ID NO:1). Recombination at these is reversible and as such variants of this sequence that facilitate irreversible recombination may be employed. The LoxP recognition site consists of the minimal 34 base sequence, ATAACTTCGTATA-NNNTANNN-TATACGAAGTTAT (SEQ ID NO:2). Recombination at these sites is also reversible and as such variants of this sequence may be used that facilitate irreversible recombination. The attB recognition sites consist of the core sequence, cCTGCTTt tTtatActAACTTGa (SEQ ID NO:3). Recombination of attB sites with attP sites is irreversible. Each pair of recombination sites is contained within an intronic sequence. Recombination acceptor sites are located in pairs sufficient for specific recombination with gene fragments flanked by corresponding recombination donor sites. The combination of recombination loci within the engineered cassette is designed but not limited to FRT, loxP and attB sites or variants thereof.

The synthesized gene fragment harboring the recombination locus is introduced into HEK293 cells via nucleofection in combination with guide RNA targeting the AAVS1 locus or other safe harbor locus. AAVS1 guide RNA targets Cas9 to introduce a double-strand break at a position within the AAVS1 locus that corresponds to the region of homology arms flanking the synthesized recombination locus fragment. The engineered locus is incorporated into the AAVS1 site via homology directed DNA repair. Cells are passaged into single cell clones and the incorporation of the engineered locus is assessed by Next Generation Sequencing (NGS). Clonal cell populations that have incorporated the recombination locus into one of the two AAVS1 genomic loci (heterozygous incorporation) are suitable for use in recombinase mediated protein diversification.

### B. Preparation of Gene Fragment Library:

For any protein of interest for which structural data is unavailable, the genomic sequence consisting of introns and exons can be computationally segregated into any number of gene fragments. In this Example, a schematic of which is shown in FIG. 13, the genomic sequence is divided to yield three fragments of approximately equal size. Gene fragment sequences are flanked by introns and donor recombination sites such that they can be inserted by recombinase activity between corresponding recombination acceptor sites in the engineered locus. Gene fragments are designed such that insertion into the recombination locus will retain the sequential order of gene fragments from the endogenous genomic sequence upon integration. Alternatively, gene fragments are designed to facilitate circular permutation of the amino acid sequence of the protein target (Yu et al., Trends in Biotechnology (2011) 29:18-25).

Gene fragments can be synthesized (e.g., commercially) as double-stranded DNA and then cloned into a plasmid backbone for delivery into cells by transfection. Methods for design and preparation of the gene fragment library can be developed to facilitate the engineering of gene fragment libraries of increasing diversity as delineated in Examples 4C and 4D, below.

### C. Rational Design and Preparation of a Gene Fragment Library:

This Example illustrates the preparation of a suitable DNA library for protein domain shuffling for a given protein when 3D structural information is available. This rational design Example combines DNA from two or more protein homologues into one new protein chimera entity. Rational design of a protein chimera is not limited to protein homologues and can be extended to protein orthologues and even unrelated protein domains. A target protein is selected (from the list outlined in Example 4G). If a protein structure for the target protein is available, the structure is used in the library design. If no structural data is available but the structure of a protein homologue is available, the homologous structure is used to build an approximate structural model (using a computer program, such as but not limited to, the program MODELLER) of the target protein or sub-domains of interest.

The target protein sequence is aligned with other homologous protein sequences in an alignment program (for example using ClustalO or Jalview). Using structural information, secondary structure predictions and the sequence alignment, the protein is computationally “cut” into segments. Criteria for suitable “cut sites” include but are not limited to: the beginning or end of domains or secondary structure elements, at the beginning or end of alpha helices, at the beginning or the end of loops, at the beginning or the end of beta strands. Prediction of contiguous (SCHEMA, Endelmann et al., Protein Engineering, Design & Selection (2004) 17: 589-594) or non-contiguous protein fragments (Smith et al., Protein Science (2013) 22:231-238) are known in the art. DNA gene fragments designed via this method are then synthesized by a manufacturer (e.g. TWIST Biosciences (San Francisco, Calif.); Agilent Technologies (Santa Clara, Calif.); Synthego (Redwood City, Calif.)) and cloned into a suitable vector for protein expression, further recombination or viral integration into a host genome (such as that of a mammalian, yeast or bacterial cell).

### D. Stochastic Preparation of a Gene Fragment Library by Enzymatic Digest of Target Protein Coding DNA:

This Example illustrates the preparation of a suitable DNA library for protein family shuffling when 3D structural information is not available that will combine DNA from two or more protein homologues into one new protein chimera entity. The library design and choice of restriction enzymes for family shuffling are known in the art (see. e.g., Crameri et al., Nature (1998) 391:288-291, reviewed in Huang et al., BioTechniques (2016) 60:91-94 2016). In short, the target protein and one or more homologues are chosen. The respective DNA sequences are obtained conveniently from manufacturers (e.g. TWIST Biosciences (San Francisco, Calif.); Agilent Technologies (Santa Clara, Calif.); Synthego (Redwood City, Calif.)) or from cDNA libraries (e.g., from ThermoFisher Scientific (Waltham, Mass.) or GenScript (Piscataway, N.J.)), or cloned from cDNA obtained from cells, or cloned from genomic DNA, using methods well known in the art. The DNA sequences are fragmented into smaller DNA pieces of variable size with a suitable restriction enzyme (e.g. DNasel or EcoRI). The fragmented DNA pieces of two or more homologous sequences are then recombined using primerless PCR. The recombined chimeric DNA sequences are then cloned into a suitable vector for protein expression or further homologous recombination or viral integration into a host genome (such as mammalian cell, yeast cell, bacterial cell).

### E. Preparation of Recombinase Expression Vectors:

The open reading frames encoding codon-optimized recombinases such as Flp, Cre or psi C31 are cloned into suitable expression vectors, such as lentiviral expression vectors, for expression in mammalian cells. Flp recombinase expression will drive recombination between FRT sites. Cre recombinase expression will drive recombination between LoxP sites and psi C31 recombinase will drive recombination between Att sites. This Example makes use of Flp, Cre and psi C31 phage-derived recombinases but can be extended to utilize known transposase or integrase enzymes in combination with alternate recognition sites.

### F. Recombinase Mediated Protein Diversification:

The gene fragment library and recombinase expression vectors prepared in Examples 4B-4E above, are introduced into the engineered protein diversification cell line prepared in Example 4A above via transfection or viral transduction. Within 24 hours, recombinase expression takes place and gene fragments undergo site-specific insertion from the gene fragment donor plasmid library. By 72 hours post introduction of the gene fragment library, the engineered recombination locus is actively transcribed into an RNA molecule from the gene fragments inserted. RNA splicing will remove introns including those in which the recombinase acceptor sites are nested. Gene fragments inserted will thus yield a mature RNA in which coding exons of each gene fragment are sequentially joined in the designated order. This mature RNA is translated into protein for assessment in downstream functional assays. The engineered recombination locus is utilized in combination with expanded libraries of variants for each gene fragment. Stochastic integration of a variant from each gene fragment library, as described in Examples 2 and 3, with each fragment in an engineered 5′ to 3′ order defined by recombination sites, yields protein diversification with functional protein configuration.

### G. Protein Targets for Protein Engineering Protocols:

Target proteins for protein engineering include but are not limited to mammalian antibodies (ABs) (IgG, IgA, IgM, IgE), antibody fragments such as Fc regions, antibody Fab regions, antibody heavy chains, antibody light chains, antibody CDRs, nanobodies, chimeric antibodies and other IgG domains; T cell receptors (TCRs); endonucleases and exonucleases, such as TALENs, CRISPR nucleases such as Cas9 and Cas3, ZFNs, meganucleases, nuclease domains such as HNH domain, RuvC domain; recombinases such as Cre, Tre, Brec1, Flp, γ-integrase, IntI4 integrase, XerD recombinase, HP1 integrase; DNA topoisomerases; transposons such as the Tc1/mariner family, Tol2, piggyBac, Sleeping beauty; RAG proteins; retrotransposons such as LTR-retrotransposons and non-LTR retrotransposons (Alu, SINE, LINE); enzymes including but not limited to arginases, glycosydases, proteases, kinases, and glycosylation enzymes such as glycosyltransferase; anticoagulants such as protein C, Protein S and antithrombin; coagulants such as thrombin; nucleases such as DNAses, RNAses, helicases, GTPases; DNA or RNA binding proteins; cell penetrating peptides and their fusions with cargo proteins; membrane proteins such as GPCRs, pain receptors such as TRP channels and ion channels; cell surface receptors including but not limited to EGFR, FGFR, VEGFR, IGFR and ephrin receptor; cell adhesion molecules like integrins and cadherins; ion channels; rhodopsins; immunoreceptors such as CD28, CD80, PD-1, PD-L1, CTLA-4, CXCR4, CXCR5, B2M, TRACA, TRBC; secreted proteins including but not limited to hormones, cytokines, growth factors; vaccine antigens such as HIV, Dengue, CMV, Ebola, Zika; viral proteins such as from AAV, lentivirus, HIV, and oncolytic viruses; snake toxin proteins and peptides including but not limited to phospholipases and metalloproteases; ribosomal cyclic peptides.

### Example 5

### T Cell Receptor Engineering

This Example illustrates methods for reprogramming the specificity of the endogenous T Cell Receptor (TCR). The method allows for re-directing TCR specificity while maintaining cell surface expression and function.

### A. Design of Guides to TCR Variable Region:

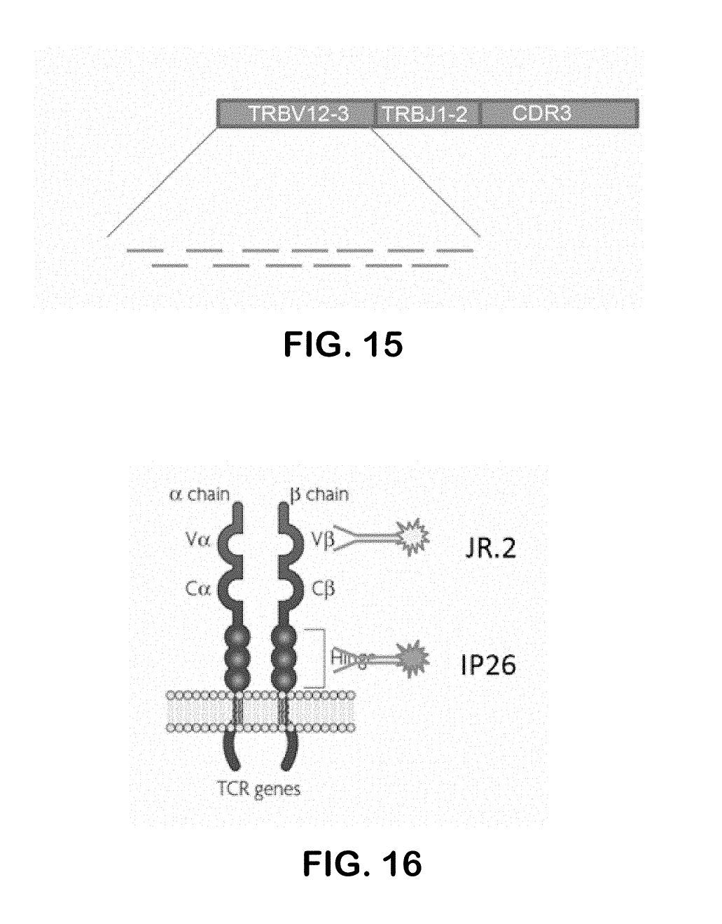

Forty-one guide RNAs (Table 1) were designed to target the coding region of the variable region of the β chain of the TCR in Jurkat T cells, which is specified with the TRVB12-3 gene (FIG. 15). The guides were designed as crRNA/trRNA pairs and chemically synthesized (Synthego, Redwood City, Calif.).

TABLE 1
Guide sequences targeting TRVB 12-3

| Target | | hg38 chromosomal | SEQ ID |
|---|---|---|---|
| name | Target sequence | coordinate | NO. |
| A1 | TATCCAGTCACCCCGCCATGAGG | chr7:142560653-142560675 | 4 |
| B1 | CCAGTCACCCCGCCATGAGGTGA | chr7:142560656-142560678 | 5 |
| C1 | CCCCGCCATGAGGTGACAGAGAT | chr7:142560663-142560685 | 6 |
| D1 | CCCGCCATGAGGTGACAGAGATG | chr7:142560664-142560686 | 7 |
| E1 | CCGCCATGAGGTGACAGAGATGG | chr7:142560665-142560687 | 8 |
| F1 | CCGCCATGAGGTGACAGAGATGG | chr7:142560665-142560687 | 9 |
| G1 | CGCCATGAGGTGACAGAGATGGG | chr7:142560666-142560688 | 10 |
| H1 | CCATGAGGTGACAGAGATGGGAC | chr7:142560668-142560690 | 11 |
| A2 | CTGAGATGTAAACCAATTTCAGG | chr7:142560702-142560724 | 12 |
| B2 | CCAATTTCAGGCCACAACTCCCT | chr7:142560714-142560736 | 13 |
| C2 | CAGGCCACAACTCCCTTTTCTGG | chr7:142560721-142560743 | 14 |
| D2 | CCACAACTCCCTTTTCTGGTACA | chr7:142560725-142560747 | 15 |
| E2 | CCCTTTTCTGGTACAGACAGACC | chr7:142560733-142560755 | 16 |
| F2 | CCTTTTCTGGTACAGACAGACCA | chr7:142560734-142560756 | 17 |
| G2 | GGTACAGACAGACCATGATGCGG | chr7:142560742-142560764 | 18 |
| H2 | GTACAGACAGACCATGATGCGGG | chr7:142560743-142560765 | 19 |
| A3 | TACAGACAGACCATGATGCGGGG | chr7:142560744-142560766 | 20 |
| B3 | ACAGACCATGATGCGGGGACTGG | chr7:142560749-142560771 | 21 |
| C3 | CCATGATGCGGGGACTGGAGTTG | chr7:142560754-142560776 | 22 |
| D3 | AACGTTCCGATAGATGATTCAGG | chr7:142560795-142560817 | 23 |
| E3 | ACGTTCCGATAGATGATTCAGGG | chr7:142560796-142560818 | 24 |
| F3 | CCGATAGATGATTCAGGGATGCC | chr7:142560801-142560823 | 25 |
| G3 | AGATGATTCAGGGATGCCCGAGG | chr7:142560806-142560828 | 26 |
| H3 | CCCGAGGATCGATTCTCAGCTAA | chr7:142560822-142560844 | 27 |
| A4 | CCGAGGATCGATTCTCAGCTAAG | chr7:142560823-142560845 | 28 |
| B4 | CCTAATGCATCATTCTCCACTCT | chr7:142560849-142560871 | 29 |
| C4 | CCACTCTGAAGATCCAGCCCTCA | chr7:142560865-142560887 | 30 |
| D4 | AGATCCAGCCCTCAGAACCCAGG | chr7:142560874-142560896 | 31 |
| E4 | GATCCAGCCCTCAGAACCCAGGG | chr7:142560875-142560897 | 32 |
| F4 | CCAGCCCTCAGAACCCAGGGACT | chr7:142560878-142560900 | 33 |
| G4 | CCCTCAGAACCCAGGGACTCAGC | chr7:142560882-142560904 | 34 |
| H4 | CCTCAGAACCCAGGGACTCAGCT | chr7:142560883-142560905 | 35 |
| A5 | CCCAGGGACTCAGCTGTGTACTT | chr7:142560891-142560913 | 36 |
| B5 | CCAGGGACTCAGCTGTGTACTTC | chr7:142560892-142560914 | 37 |
| C5 | CCTCCAGGCTGTGAGCACAGATA | chr7:142796834-142796856 | 38 |
| D5 | CCAGGCTGTGAGCACAGATACGC | chr7:142796837-142796859 | 39 |
| E5 | AGCACAGATACGCAGTATTTTGG | chr7:142796847-142796869 | 40 |
| F5 | GATACGCAGTATTTTGGCCCAGG | chr7:142796853-142796875 | 41 |
| G5 | AGTATTTTGGCCCAGGCACCCGG | chr7:142796860-142796882 | 42 |
| H5 | CCCAGGCACCCGGCTGACAGTGC | chr7:142796870-142796892 | 43 |
| A6 | CCAGGCACCCGGCTGACAGTGCT | chr7:142796871-142796893 | 44 |

### B. Transfection of Guides in Jurkat T Cells:

60 pmol each crRNA and trRNA was complexed with 20 pmol Cas9 to create RNPs. RNPs were electroporated into Jurkat T cells using the 96-well Shuttle system (Lonza, Allendale, N.J.). For each target, 200,000 Jurkat T cells were mixed with 2.2 μl RNP in 20 μl Nucleofector SE Buffer™ (Lonza, Allendale, N.J.) and electroporated using Nucleofector Program 96CL-120™. 80 μl RPMI media+10% FBS was added to each well. 50 μl cell suspension was transferred to 2 96 well plates pre-filled with 150 μl/well RMPI media+10% FBS. Cells were cultured at 37° C. and 5% CO2.

C. Generation of Clonal Cell Lines with Reprogrammed TCRs:

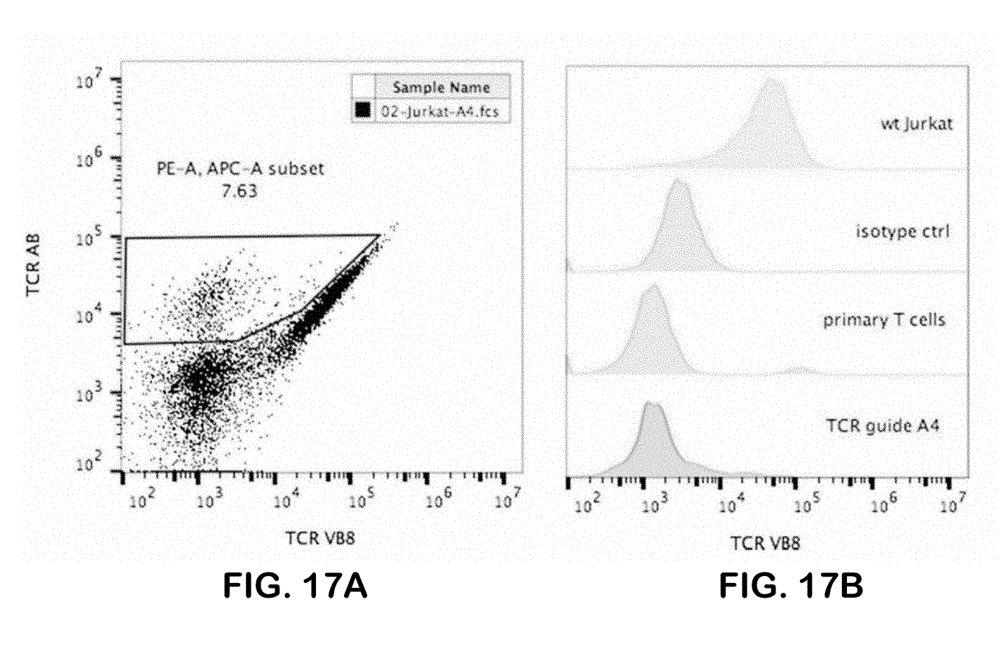

96 hours after transfection, cells were screened for reprogrammed TCRs using flow cytometry. Cells from each well were transferred to V-bottom plates, spun down and resuspended in FACS buffer plus 0.5 μl TCR antibody, antibody clone IP-26 (Biolegend, San Diego, Calif.) and 0.5 μl TCRVB8 antibody clone JR.2 (Biolegend, San Diego, Calif.). The IP26 antibody recognizes an epitope in the constant region of the TCR complex while the JR.2 antibody recognizes an epitope in the variable region of the TCR β chain (FIG. 16). Using these antibodies, cells were screened for high binding of IP26 and reduced binding of JR.2 compared to wild-type Jurkat cells (FIG. 17A and FIG. 17B).

Six of 41 transfected wells had populations meeting the screening criteria. The targets with hits were: D3, E3, F3, G3, H3, A4 (see Table 1). These 6 edited cell populations were scaled up from 96 well to 2 ml cultures.

To generate clonal cell lines, cells were sorted using the SH800Z Cell Sorter (Sony, Tokyo, Japan). Cells were stained with IP26 and JR.2 antibodies as above, and gates for IP26 positive and JR.2 negative were set using wild-type controls. Single cells were sorted into 96 well plates and allowed to expand for 3 weeks.

### D. Genotyping Clonal Cell Lines:

After 3 weeks of cell culture, gDNA was harvested using Quick Extract (Epicentre, Madison, Wis.) to determine the genotype of the clonal cell lines. Primers were designed to amplify the region targeted by the guides used to generate each clonal cell line (Table 2). All six of the selected targets used the same primers. Using the isolated gDNA, a first PCR was performed using Q5 Hot Start High-Fidelity 2× Master Mix (New England Biolabs, Ipswich, Mass.) at 1× concentration, primers at 0.5 μM each, 3.75 μL of gDNA in a final volume of 10 μL and amplified 98° C. for 1 minute, 35 cycles of 10 seconds at 98° C., 20 seconds at 60° C., 30 seconds at 72° C., and a final extension at 72° C. for 2 minutes. Each PCR reaction was diluted 1:100 in water.

TABLE 2
Primers to amplify targeted regions of TRBV12-3

| | | Amplicon |
|---|---|---|
| F primer | R primer | coordinates |
| AGGCCACAACTCCCTTTTCT | GGCTGGATCTTCAGAGTGGA | chr7:142560722- |
| (SEQ ID NO: 45) | (SEQ ID NO: 46) | 142560883 |

All the PCR reactions were pooled and transferred into a single microfuge (“amplicon library”) tube for SPRIselect™ bead-based cleanup (Beckman Coulter, Pasadena, Calif.) of amplicons for sequencing. To each tube, 0.9× volumes of SPRIselect™ beads were added, mixed, and incubated at room temperature for 10 minutes. The microfuge tube was placed on magnetic tube stand (Beckman Coulter, Pasadena, Calif.) until the solution clears. Supernatant was removed and discarded, and the residual beads were washed with 1 volume of 85% ethanol, and incubated at room temperature for 30 seconds. After incubation, ethanol was aspirated and beads were air dried at room temperature for 10 minutes. Each microfuge tube was removed from the magnetic stand and 0.25× volumes of Qiagen EB™ buffer (Qiagen, Venlo, Netherlands) were added to the beads, mixed vigorously, and incubated for 2 minutes at room temperature. Each microfuge tube was returned to the magnet, incubated until the solution had cleared, and then the supernatant containing the purified amplicons was dispensed into a clean microfuge tube. The purified amplicon library was quantified using the Nanodrop™ 2000™ System (Thermo Scientific, Wilmington, Del.) and library quality was analyzed using the Fragment Analyzer™ System (Advanced Analytical Technologies, Ames, Iowa) and the DNF-910 Double-stranded DNA Reagent Kit™ (Advanced Analytical Technologies, Ames, Iowa).

For next generation sequencing, the pooled amplicon library was normalized to a 4 nM concentration as calculated from quantified values and the average size of the amplicons. The amplicon library was analyzed on MiSeq Sequencer™ (Illumina, San Diego, Calif.) with MiSeq Reagent Kit v2™ (Illumina, San Diego, Calif.) for 300 cycles with two 151-cycle paired-end runs plus two eight-cycle index reads.

### E. Analysis of Sequence Data:

The identities of products in the sequencing data were determined based on the index barcode sequences adapted onto the amplicons in the barcoding PCR. A computational script was to process the MiSeq™ data that executes, for example, the following tasks:
Reads were aligned to the human genome (build GRCh38/38) using Bowtie (bowtie-bio.sourceforge.net/index.shtml) software.
Aligned reads were compared to the expected wild-type locus region
Locus sequence and reads not aligning to any part of the target locus were discarded.
Reads matching wild-type target locus sequences were tallied.
Reads with indels (insertion or deletion of bases) were categorized by indel type and tallied.

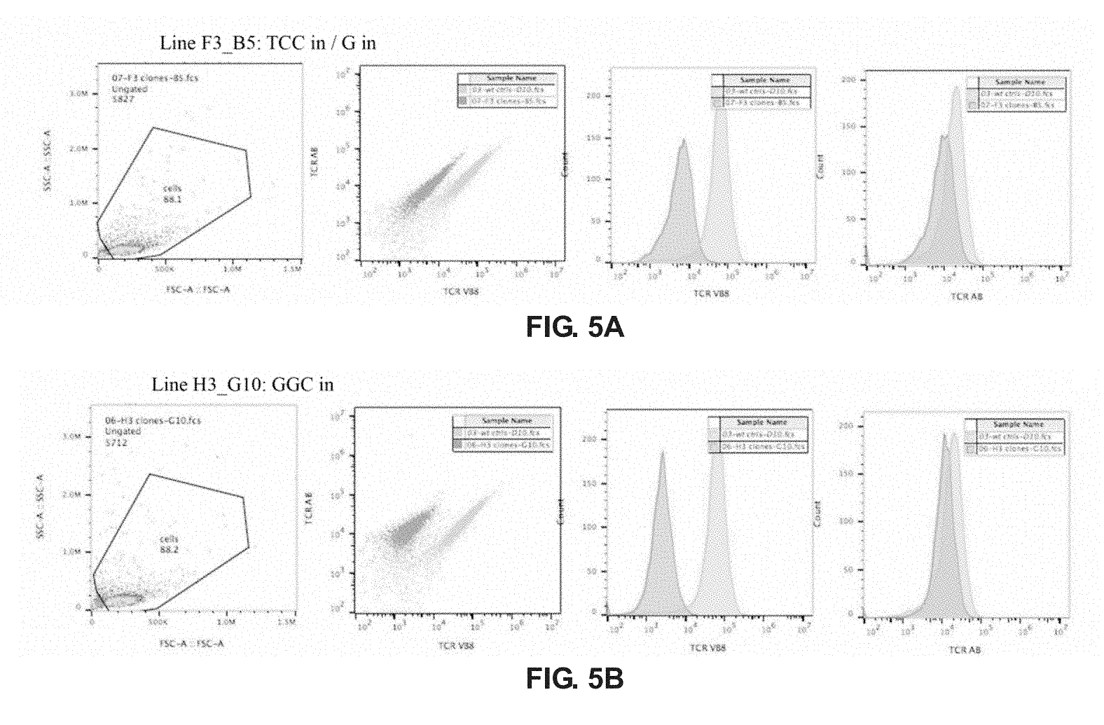

Sequence analysis of clonal cell lines revealed a mix of in-frame and out-of-frame mutations. All lines were screened with IP26 and JR.2 antibodies as above to confirm the TCR binding phenotype. Examples of genotypes and phenotypes for 2 clonal cell lines is shown in FIG. 5A (Line F3_B5) and FIG. 5B (Line H3_G10). Line F3_B5 has a 3 base insertion in one allele and 1 base insertion in the other, which results in reduced JR.2 binding compared to wild-type, comparing the dark grey versus light grey histograms in the third panel from the left. Line H3_G10 is homozygous with a three base insertion in TCRVB12 which results in reduced JR.2 antibody binding. Both lines have normal IP26 antibody binding, indicating normal TCR complex formation on the cell surface (right hand panels).

F. Quantification of IL-2 Secretion from Clonal Cell Lines:

TCR expression was confirmed using IP26 antibody staining and loss of TCR chain variable region specificity was confirmed with JR.2 antibody staining. All cell lines were cryopreserved and some cell lines were selected for functional analyses.

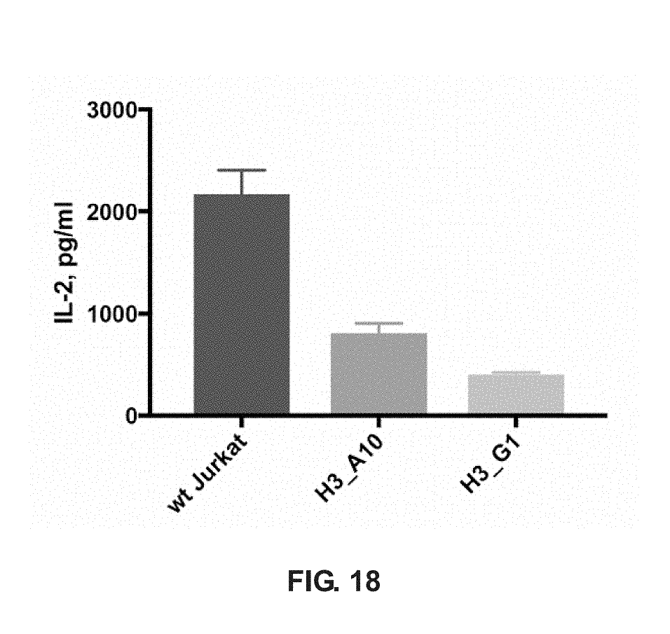

Wild-type and engineered Jurkat cell lines were stimulated with 1 ng/ml PMA and 250 ng/ml A23187 for 24 hours. Supernatants were harvested and IL-2 concentration was quantified using Human IL-2 DuoSet ELISA DY202-05™ (R&D Systems, Minneapolis, Minn.) according to manufacturer's instructions. Two engineered cell lines secreted IL-2 in response to TCR stimulation (FIG. 18).

Although preferred embodiments of the subject methods have been described in some detail, it is understood that obvious variations can be made without departing from the spirit and the scope of the methods as defined by the appended claims.

1-25. (canceled)

26. A method for engineering a T cell receptor (TCR) protein comprising a trait of interest, the method comprising:
introducing into human lymphoblastic cells a programmable endonuclease and one or more DNA binding molecules that targets nucleotide sequences in a TCR protein coding sequence, thereby producing one or more double-strand breaks (ds-breaks) in the TCR protein coding sequence, which ds-breaks are repaired by DNA repair pathways of the human lymphoblastic cells, whereby human lymphoblastic cells comprising a DNA library comprising mutated TCR protein coding sequences are produced; and
screening the library for cells that express the engineered TCR protein comprising a mutated TCR protein coding sequence comprising the trait of interest.

27. The method of claim 26, wherein the human lymphoblastic cells comprise Jurkat cells or CCRF-CEM cells.

28. The method of claim 26, wherein the TCR protein coding sequence comprises a TCRα chain and/or TCRβ chain.

29. The method of claim 26, wherein the DNA binding molecules target a complementary determining region of the TCR protein coding sequence selected from the group consisting of a complementary determining region 1 (CDR1), a complementary determining region 2 (CDR2), a complementary determining region 3 (CDR3), and combinations thereof.

30. (canceled)

31. The method of claim 44, wherein the DNA binding molecule comprises a guide polynucleotide and the programmable endonuclease comprises a Cas9 endonuclease.

32. The method of claim 31, wherein the human lymphoblastic cells constitutively express the Cas9 endonuclease.

33. The method of claim 31, wherein the introducing comprises introducing a complex comprising the Cas9 endonuclease and the guide polynucleotide into the human lymphoblastic cells.

34. The method of claim 33, wherein the DNA binding molecule comprises a single-guide RNA (sgRNA).

35-43. (canceled)

44. The method of claim 26, wherein the DNA binding molecule and the programmable endonuclease comprise a CRISPR-Cas system.

45. The method of claim 31, wherein the Cas9 endonuclease comprises a Streptococcus pyogenes Cas9 endonuclease.

46. The method of claim 44, wherein the DNA binding molecule comprises a guide polynucleotide and the programmable endonuclease comprises a Cpf1 endonuclease.

47. The method of claim 44, wherein the DNA binding molecule comprises a guide polynucleotide and the programmable endonuclease comprises a Class 1 Type I multiprotein complex.

48. The method of claim 47, wherein the Class 1 Type I multiprotein complex comprises a CASCADE complex.

49. The method of claim 26, wherein the introducing further comprises introducing one or more oligonucleotides comprising 3-50 base pairs.

50. The method of claim 28, wherein the DNA binding molecules target a complementary determining region of the TCR protein coding sequence selected from the group consisting of a complementary determining region 1 (CDR1), a complementary determining region 2 (CDR2), a complementary determining region 3 (CDR3), and combinations thereof.

51. The method of claim 50, wherein the DNA binding molecule and the programmable endonuclease comprise a CRISPR-Cas system.

52. The method of claim 51, wherein the DNA binding molecule comprises a guide polynucleotide and the programmable endonuclease comprises a Cas9 endonuclease.

53. The method of claim 51, wherein the DNA binding molecule comprises a guide polynucleotide and the programmable endonuclease comprises a Cpf1 endonuclease.

54. The method of claim 51, wherein the DNA binding molecule comprises a guide polynucleotide and the programmable endonuclease comprises a Class 1 Type I multiprotein complex.

55. The method of claim 54, wherein the Class 1 Type I multiprotein complex comprises a CASCADE complex.
